(19) United States
(12) Patent Application Publication
(10) Pub. No.: US 2014/0128277 A1
(43) Pub. Date: May 8, 2014

(54) Method for Identifying a Subset of Polynucleotides from an Initial Set of Polynucleotides Corresponding to the Human Genome for the In Vitro Determination of the Severity of the Host Response of a Patient

(71) Applicant: Analytik Jena AG, Jena (DE)

(72) Inventors: Eva Möller, Jena (DE); Andriy Ruryk, Jena (DE); Britta Wlotzka, Erfurt (DE); Cristina Guillen, Jena (DE); Karen Felsmann, Jena (DE)

(73) Assignee: Analytik Jena AG, Jena (DE)

(21) Appl. No.: 14/003,646

(22) PCT Filed: Mar. 7, 2012
(86) PCT No.: PCT/EP2012/053870

§ 371 Date: Jan. 9, 2014

(30) Foreign Application Priority Data
Mar. 8, 2011 (DE) 10 2011 005 235.6

(51) Int. Cl. C12Q 1/68

(52) U.S. Cl. CPC C12Q 1/6883
USPC 506/9; 435/6.12

(57) ABSTRACT

The invention discloses a method for identifying a subset of polynucleotides from an initial set of polynucleotides corresponding to the human genome for the in vitro determination of severity of the host response of a patient having a severe infectious and/or inflammatory condition, in a sample, a measuring device comprising a plurality of different gene probes which represent the entire human genome, the test persons, depending on their infectious and/or inflammatory status, are divided into at least two clinically determined phenotype groups, changes of the gene expression signals between the phenotype groups are compared statistically, and gene probes are selected based on the gene expression signals which have significantly changed statistically between at least two phenotype groups.

This application is a United States National Stage Application claiming the benefit of priority under 35 U.S.C. 371 from International Patent Application No. PCT/EP2012/053870 filed Mar. 8, 2012, which claims the benefit of priority from German Patent Application Serial No. DE 10 2011 005 235.6 filed Mar. 8, 2011, the entire contents of which are herein incorporated by reference.

The present invention relates to a method for identifying a subset of polynucleotides from an initial set of polynucleotides corresponding to the human genome for the in vitro determination of the severity of the host response of a patient being in a severe infectious and/or severe inflammatory condition. The invention further relates to the method of use of k tuples of polynucleotides selected from a group consisting of m polynucleotides having SEQ ID No: 1 to SEQ ID No: 7704, k being at least 7 and equal to or smaller than the number of polynucleotides m in the group, for determining a score as measurement for the severity of the host response of a test person being in a severe infectious and/or severe inflammatory condition. The invention further relates to the method of use of polynucleotides for performing the method in accordance with the invention. The invention likewise relates to the use of protein gene products from polynucleotides.

Sepsis (blood poisoning) is a life-threatening infection which can affect the entire body. It is associated with high mortality, is becoming increasingly prevalent and affects people at any age. Sepsis endangers medical process in many areas of high performance medicine and consumes a large portion of health care resources. Mortality of severe sepsis has not improved considerably in recent decades. The last two steps in innovation after introducing blood culture (around 1880) were the introduction of antibiotics over 60 years ago and the beginning of intensive care about 50 years ago. To achieve a similarly significant advance in treatment today, new types of diagnosis must be made available.

In international literature the criteria established at a consensus conference of the “American College of Chest Physicians/Society of Critical Care Medicine Consensus Conference (ACCP/SCCM)” of 1992 have gained broadest acceptance for defining the term sepsis [Bone et al., 1992]. The international sepsis conference of 2001 moreover proposed a new concept (named PIRO) for describing sepsis, which concept is composed of the criteria of predisposition, infection, immune response (response) and organ dysfunction [Levy et al., 2003]. Despite a new definition of SIRS/sepsis by the acronym PIRO [Opal et al., 2005] most studies still use the ACCP/SCCM consensus conference of 1992 [Bone et al., 1992] to classify their patients.

Life-threatening bacterial infections and the consequences thereof, i.e. sepsis and consecutive organ failure, are frequent complications in hospital patients and increase worldwide by 2% to 7% per year. In Germany, of roughly 154,000 ill persons 60,000 people die of severe sepsis, which thus is one of the most frequent causes of death in intensive care units. A specific antibiotics therapy started within the first few hours after the infection is considered to be crucial for successful treatment [Ibrahim et al., 2000; Fine et al., 2002; Garracho-Montero et al., 2003; Valles et al., 2003]. With 40% to 60% the mortality rate at present is unacceptably high, the risk in case of a delayed antibiotic therapy based on resistance test results rising considerably. The specific detection of the pathogen in sepsis at present is unsatisfactory in clinical application. The “gold standard” of blood culture suffers from lacking sensitivity and remains negative in 80% to 90% of all cases of sepsis. Moreover, the results of the blood culture only are provided after 24 to 72 hours and then merely form the basis of further microbial diagnostics (species differentiation, creation of an antibiogram). Frequently, a therapy using broad-spectrum antibiotics will be initiated at the time of a first suspicion of sepsis without safe microbiological results. On the one hand, this fosters the development of multi-resistant germs, on the other hand, antibiotic pre-treatment reduces the success rate of a blood culture taken at a later stage. Epidemiological data prove that in case of an inadequate therapy redoubled mortality [Valles et al., 2003], and in case of a delayed effective start of a therapy, an increase in mortality by more than 5% per hour is to be expected [Iregui et al., 2002; Ibrahim et al., 2000; Fine et al., 2002; Garnacho-Montero et al., 2003; Valles et al., 2003; Kumar et al., 2006].

Exact diagnosis of systemic inflammatory and infectious disease-related conditions and their causes and associated risks for subsequent complications is playing an important role for the clinical decisions for treating patients and subsequent follow-up observation not only in sepsis, but also in a number of other indications. In this context, the treatment of acute and chronically ill patients and perioperative monitoring are to be seen. It is known that in case of acute pancreatitis an infection significantly worsens the prognosis of a lethal outcome by 16% to 40%. In the event of a complex super-infection there is an elevated risk of sepsis with a mortality of up to 90%. Furthermore, the follow-up observation of intra-abdominal inflammation and/or infection in chronically ill, postoperative and trauma patients is important. There are difficulties even today of a clear clinical diagnosis of intra-abdominal infections. Follow-up monitoring of chronically ill persons, such as patients with liver cirrhosis or renal failure, is of clinical relevance since those patients may be predestined, depending on organ decompensation, to take an inflammatory and/or infectious course of disease. In particular, renal failure patients with peritoneal dialysis are prone to chronic inflammations and infections [Blake, 2008]. Of particular interest is the observation of patients with liver cirrhosis as those patients may spontaneously develop bacterial peritonitis, which has a high mortality [Koulaouzidis et al., 2009]. The diagnosis of secondary peritonitis within the scope of postoperative treatment is of great clinical value and may and may greatly influence the success of surgery. Postoperative infections still are a major problem today in surgical treatment. One percent of laparatomies carried out result in complications after surgery. Here, the complication rates between the surgical procedures may vary considerably. In particular, insufficient suturing may result, in operations on the gastro-intestinal tract, in fulminant spread of bacteria into the sterile abdominal cavity.

Infectious courses play a role, among other things, in post-surgery follow-up treatment after transplantations, thoracotomies, limb and joint corrections and neurosurgical operations.

The person of ordinary skill in the art is aware that these examples are merely illustrative and that there are numerous other fields of application for which the identification and observation of the course of an infectious inflammatory process and assessment of its severity and the resulting risk of corresponding complications are of great importance. The present invention provides a solution to this diagnostic problem.

The morbidity and contribution to mortality of SIRS and sepsis is of interdisciplinary clinical and medical importance as this will, increasingly, put at risk the gains in treatment results achieved in advanced therapeutic methods in numerous medical fields (such as. e.g. traumatology, neurosurgery, heart and lung surgery, visceral surgery, transplantation medicine, hematology/oncology, etc.) which without exception intrinsically bear an increased disease risk for SIRS and sepsis on account of unbalanced and uncontrolled infectious-inflammatory processes. This also is reflected in the frequency of sepsis rising steadily: from 1979 to 1987, an increase by 139%, i.e. from 73.6 to 176 cases of illness per 100,000 hospital patients, was recorded [MMWR Morb Mortal Wkly Rep 1990]. The reduction of morbidity and mortality for a large number of seriously ill patients therefore is tied to simultaneous progress in the prevention, treatment and in particular detection and follow-up monitoring of sepsis and severe sepsis.

Particularly in patients that have overcome an initial fulminant systemic inflammatory response to highly virulent pathogens, there is long phase (of up to several weeks) of an increased risk for sepsis-induced multi-organ failure. At present, induced immune suppression is discussed as being the cause of such risk. In addition to the immune system being incapable of neutralizing a primary infection, intensive care patients quite frequently develop secondary infections with multiple antibiotic-resistant and little virulent germs or there is an onset of latent viral infections [Hotchkiss et al. 2009, 2010], resulting in high clinical relevance, particularly in view of an increasing development of resistance against antibiotics and the lack of new effective antimicrobial agents. An important object of clinical research therefore is the prevention of such sepsis-induced immunosuppression. Molecular targets for intervention in form of a molecular immune therapy are well known (such as IL-15 and IL-7 as anti-apoptotic and immuno-stimulatory cytokines); however, first clinical tests reveal that the therapy decision should be based on the individual immune status. Tight monitoring of innate and adaptive immune functions in further studies requires new and more complex measuring instruments for immunosuppressed patients in order to shift the balance of pro- and anti-inflammatory signals to the benefit of the patients.

The following mechanisms are presently considered as mechanisms of immunosuppression:
Production of anti-inflammatory cytokines, e.g. IL-10; by this, a development of so-called T-cell anergy may be induced (non-responsive behavior)
Die-off of immunocompetent cells
   Apoptoptic depletion of immunoeffector cells (e.g. lymphocytes and dendritic cells)
   The restoration of a normal population of specific cells obviously is tied to an improved prediction
Suppression of MHC-ClassII molecules (Suppression of the induction of an adaptive immune response)
Expression of negative co-stimulatory molecules (PD-1, CTLA-4)

A study [Meisel et al 2009] provides the first example for therapeutic intervention in immunosuppressed patients. Molecular surface markers of monocytes were utilized (HLA-DR) in order to gain a statement on the immune status. A strongly reduced population in monocytes is a characteristic indication for sepsis-associated immunosuppression (also shown in Venet et al. 2010). In case of a minor HLA-DR expression in the blood of patients, the patients were treated with the growth factor GM-CSF or with a placebo. GM-CSF has strong immunostimulatory properties; in particular, phagocytosis, proliferation, and the pathogen defense of neutrophils and monocytes/macrophages are stimulated. Previous studies revealed that immunostimulants may reverse a long-lasting deactivation of monocytes. As a result of the study, an increase by number could be proved for many immunocell populations: both monocytes and neutrophils and lymphocyte populations were benefitting from the treatment. The patients in the treatment group likewise exhibited improvements in their clinical state: a shorter period of ventilation and hospitalization were noticeable. For the first time, the positive influence of a biomarker-guided immunostimulatory therapy on an immunological and clinical level could be attested.

In a further study, the immunosuppressive phase of sepsis was characterized more closely [Muenzer et al. 2010]. A phase of ongoing immunosuppression follows an initial hyper-inflammatory phase that may be connected with a so-called cytokine attack, resulting early organ damage and death of the patients. The balance of the immune system hence is disturbed in both phases; the importance of an intervention in the second phase is emphasized by the occurrence of secondary infections and extremely high mortality. In a mouse model it could be demonstrated how in the course of sepsis over a period of 7 days IL-10 synthesis could be blocked using an immunomodulator and the production of pro-inflammatory cytokines could be stimulated. In particular, it was ascertained that the point of time of a so-called “second hit”, i.e. a second infection, is of crucial importance to the survival of the organism. The status of immunoparalysis in the mouse model lasted 4 days, on day 7 after the septic stimulus immune response in part was restored. This was expressed in the survival rate of the animals after a septic stimulus which was higher after 7 days than after 4 days. In a hypo-inflammatory time frame (4 days) the survival rate likewise could be increased by immunomodulator AS101 and/or by blocking IL-10. Up to the normalization of the immune status, recurrence of innate immune cells and a balance of pro- and anti-inflammatory signaling cascades there thus arises a critical gap with a high risk for further infections and survival of the patient.

A further study describes drastic changes in the populations of lymphocytes in patients suffering from a septic shock [Venet et al. 2010]. The fact that such extensive changes mostly can be detected at the time of diagnosis points out to the fact that they constitute a very early event in the chain of processes that lead to immunoparalysis and predisposition for further infections. In studies with patients the exact beginning of a septic episode cannot be defined exactly; thus, the study populations with regard to the course of the disease cannot be synchronized reliably. However, it could be ascertained that after onset of a septic shock the immunosuppressed condition continued for approximately 48 hours despite intensive care measures. Here arises an enormous need for a monitoring tool to qualify patients for immunomodulatory interventions.

The immune status of patients being diagnosed with sepsis consequently is strongly impaired. Impairment relates both to the innate and the adaptive immune system. Characteristics of such impairment are the loss of immunoeffector cells from the peripheral blood stream through apoptosis, a decline in the expression of MHCII molecules and a decline in monocytes that can be stimulated by cytokines. Such impairment of an immune condition may be reversible. The consequence of such impairment on the one hand is an insufficiency of eliminating infections and controlling the source of an infection, so that it continues to remain active. On the other hand, the probability that secondary nosocomial infections are formed is very high. Such infections frequently are caused by minor pathogenic bacteria that are no hazard in an intact immune condition.

A macroscopic post-mortem examination of 235 critically ill patients the death of which was caused by sepsis or septic shock, showed that in 80% of those cases an active infection source was ascertained [Torgersen et al., 2009]. The organs that were hit the most were the lung, abdomen, and the genitor-urinary tract. A large number of those patients was transferred to the intensive care unit on account of their being diagnosed with sepsis and treated for more than 7 days prior to their death. Such a period of time may be regarded as being sufficiently long in order to bring an infectious source under control. Although an immediate control of the infectious source in combination with antibiosis constitute the central measures of a sepsis therapy, the measures for controlling the infectious source had not been successful in the majority of patients participating in trials and appear to have been the cause of their death. The authors of the publication recommend the development of improved diagnostic and therapeutic methods in order to cope with the medical needs in this field.

A recently published study draws the conclusion that about 20% of patients admitted to hospital under suspicion of sepsis after careful examination in fact exhibit non-infectious causes of the disease the presentation of which, however, being equal to that of sepsis. The authors interpret their findings to the effect that sepsis rather comprises the continuum of a syndrome and does not constitute a definite specific disease [Heffner et al., 2010].

In a cohort of 857 patients the endotoxin level was examined on the day of their being transferred to the intensive care unit. In so doing, it was found that endotoxemia, a significantly increased level of endotoxins in the patients' blood, is widespread in critically ill patients. In more than half of all patients examined an endotoxin level was measured that was higher than two standard deviations of the value determined in healthy test persons. At the same time, a large discrepancy between a high endotoxin value and the number of confirmed infections with gram-negative pathogens was observed. It is concluded that the origin of the endotoxin is of endogenous nature and has to lie in the enteric flora, both endotoxin and viable bacteria being able to find their way into the blood stream on account of translocation processes. High endotoxin values were correlated with higher APACHE II scores and a higher prevalence of severe sepsis, so that it is assumed that endotoxemia is an indication for a high-risk sub-population in critically ill patients [Marshall et al., 2004]. Endotoxemia likewise may be regarded as being the cause of an excessive stimulation of the immune system.

A review gives an overview on clinical and immunological parameters that determine the risk of developing a septic complication with lethal consequences after serious surgical operations and trauma [Kimura et al., 2010]. The current prior art suggests that surgical operations and traumatic injuries have such heavy impact on the so-called innate and adaptive immune response that suppression of cellular immunity of the body as a result of an excessive inflammatory reaction is responsible for a high susceptibility of a subsequent septic episode. The reaction cascades of an innate and adaptive immune response are initiated and modulated by so-called Pathogen-Associated Molecular Structures (PAMPS) and tissue Damage-Associated Molecular Structures (DAMPS) through the corresponding identification receptors.

The spectrum of the incidence of disease that is thus comprised by the invention is the progression of an infectious-inflammatory reaction of the body that also is referred to as host response, from the ability of effectively combating pathogens to the suppression of immune defense, in which the pathogens persist in the location of the infection and secondary and/or nosocomial infections occur.

In the use of molecular-diagnostic DNA-based pathogen identification clinically irrelevant results such as non-illness-associated bacteremia, the presence of freely circulating bacterial and fungal nucleic acids from colonization as well as the detection of non-vital pathogen cells are problematic to assessing the result. Evidence of the presence of circulating microbial DNA from translocation processes or the transient presence of non-disease-associated bacteria in the blood was proved in vivo [Dagan et al., 1998; Isaacman et al., 1998]. The origin and clinical significance of such false positive findings are mostly unclear and could result from so far unknown interactions of a host and the pathogen [Schrenzel, 2007]. Moreover, cases are known in which bacteria were isolated from the blood of symptome-free blood donors and even transient fungemia without visible clinical significance was already reported [Davenport et al., 2007, Rodero et al., 2002].

In the “unclear” cases described above the measurement of an immune condition by definition may be used so as to more reliably assess the significance and clinical relevance of the findings resulting from DNA-based pathogen detection.

The subject matter of the invention may be summarized as follows. An excessive stimulation of the immune system through PAMPS and DAMPS, e.g. by way of an uncontrolled center of infection or excessive inflammatory occurrence after a severe surgical operation, has influence on the innate and adaptive immune system. The resulting reaction of the body, also referred to as host response, and the resulting “immune burden” depends on the extent, quantity, duration and/or frequency of an infectious and/or inflammatory stimulation. The stimulation cannot be measured directly, but as a reaction of the body to the stimulation, as severity of the host response. The reaction is subject to continuous change in form of an increase from the state of a healthy person to a maximum as is e.g. the case in the extreme example of an infection in the blood stream. As was shown in a large number of studies, the body in such a state no longer is equipped with the protective mechanisms of the immune system. Existing infections can no longer be combated effectively. There is a high risk in this phase of developing a secondary infection. This applies especially in cases in which sterile processes were the cause of inducing an immunosuppression. Clinical measures such as, for example, identifying the cause of the excessive stimulation, controlling the center of infection, surgical control of the source of an infection, specific antibiosis or preventive medicinal therapies for termination/blocking of immunosuppression have to be initiated promptly in those cases.

The invention provides a diagnostic test that may be used for determination and follow-up observation of the described process of the disease as well as for controlling the success of therapeutic measures taken.

An excessive stimulation may have the following causes:

Uncontrolled center of infection

Insult through sterile inflammatory event
Tissue damage on account of an operation
Tissue damage on account of trauma
Necrotic processes

Endotoxemia
Translocation from the intestines due to a severe pathological event

The indicated processes lead to a transient induced immunosuppression of the innate and adaptive immune system and are correlated with:
High mortality through an uncontrolled center of infection
High risk of a nosocomial or secondary infection associated with life-threatening complications

Such a pathophysiologic event has to be countered by clinical measures that are suited for the respective case:
Identification of an infection through escalation of diagnostic measures
Infectious source control, also through surgical intervention
Specific antibiosis in known pathogens
Calculated antibiosis for the prevention of a secundary infection
Immunostimulatory therapeutic measures
Anti-inflammatory therapeutic measures in sterile infectious events

Several approaches to the diagnosis of SIRS and sepsis have been developed.

One group contains scoring systems such as APACHE, SAPS and SIRS, which can stratify the patients on the basis of a wide variety of physiological indices. While in some studies a diagnostic potential could be proven for the APACHE II score, other studies have shown that APACHE II and SAPS II are not able to differentiate between sepsis and SIRS [Carrigan et al., 2004].

In their review, Pierrakos and Vincent [Pierrakos et al., 2010] summarize the state of the search for a biomarker for the indication of sepsis. 3370 publications for 178 different biomarkers were inspected. It was concluded that most biomarkers were examined predominantly in clinical trials and mainly as prognostic markers. Few were tested as diagnostic markers. None of those candidates has shown sufficient sensitivity or specificity for a routine application in hospital. None of the markers was tested for the problem of a patient's immune status. Although Procalcitonin (PCT) and C-reactive Protein (CRP) are used, they likewise exhibit only limited properties in differentiating sepsis and other inflammatory conditions, or the usefulness of predicting specific outcomes.

Procalcitonin is a 116 amino acid protein that plays a role in inflammatory responses. Despite the wide acceptance of the biomarker PCT international studies have revealed that the achieved sensitivies and specificities of the sepsis marker PCT are still insufficient, especially in differentiating between a systemically bacterial SIRS, i.e. sepsis, and a non-bacterial SIRS [Ruokonen et al., 1999; Suprin et al., 2000; Ruokonen et al., 2002; Tang et al., 2007a]. The meta-analysis by Tang and colleagues [Tang et al., 2007a] in which 18 studies were considered, shows that PCT is poorly suited to discriminate SIRS from sepsis. Moreover, the authors emphasize that PCT has a very weak diagnostic accuracy with an Odd Ratio (OR) of 7.79.

C-reactive protein (CRP) is a 224 amino acid protein that plays a role in inflammatory reactions. The CRP measurement serves as an indicator of the progress of the disease as well as to effectiveness of the chosen therapy.

Several reports have described that in the intensive care area PCT is more suited as a marker for diagnostics than CRP [Sponholz et al., 2006; Kofoed et al., 2007]. In addition, PCT is considered better suited than CRP for distinguishing a non-infectious vs. infectious SIRS and to distinguish bacterial infection vs. a viral infection [Simon et al., 2004].

It is obvious to the person of ordinary skill in the art that the solution provided with this invention can be combined with the above-indicated biomarkers such as PCT or CRP, but not limited to these, in order to expand the diagnostic value.

A further group includes biomarkers or profiles, which were identified on the transcriptome level. Gene expression profiles or classifiers are suitable for determining the severity of sepsis [WO 2004/087949], the distinction between local and systemic infection [unpublished DE 10 2007 036 678.9], identifying the source of infection [WO 2007/124820] or of gene expression signatures for distinguishing between various etiologies and pathogen-associated signatures [Ramilo et al., 2007]. However, due to insufficient specificity and sensitivity of the consensus criteria according to [Bone et al., 1992], of the currently available protein markers and also due to the time required for proof of the source of infection through blood culture, there is an urgent need for new procedures which take into consideration the complexity of the disease. Many gene expression studies that are based on either individual genes and/or combinations of genes identified as classifiers, and numerous descriptions of statistical methods to derive a score and/or index [WO 2003/084388; U.S. Pat. No. 6,960,439] are part of the prior art.

In the use of gene expression markers for identifying a pathophysiological condition the quantities of the corresponding mRNA that are present in a sample, the gene expression levels, are quantified. The information determined by such gene expression levels is the respective over- or under-expression of these mRNAs that is determined experimentally based on a control state or based on control genes. Ascertainment of an over- or under-expression may be considered analogously to determining the concentration of a protein bio marker.

Several applications of gene expression profiles are known in the prior art.

Pachot and colleagues [Pachot et al., 2006] examined whether, based on differential whole blood gene expressions, predictions for the outcome variable survival vs. non-survival in patients with septic shock may be made. For this problem they identified, by screening on an affimetrix array, a signature of 28 differentially expressed genes. In a very small test data set they demonstrated that by this signature, survivor and non-survivor can be differentiated with high sensitivity and specificity. For plausibility of their result they reason that the late phase of a septic shock is characterized by the development of an immunosuppressed condition and that restoration of the immune function is necessary for the survival of the patients. In this respect, they state that a number of over-expressed genes in survivors are to be allocated to the innate immune system and explain the ascertained over-expression by a recovery of the immune system.

US 2008/0020379 A1 relates to the diagnosis and prognosis of infectious diseases, clinical phenotypes and other physiological conditions, thereby using host gene expression biomarkers in blood.

According to the abstract, the point of US 2008/0020379 A1 is that specific sets of gene expression markers from peripheral blood (leukocytes) may be an indication to a host response to exposure, response and recovery of infectious pathogen infections.

US 2008/0020379 A1 merely generally points out to the fact that by using the unique technique disclosed in US 2008/0020379 A1 it is possible to diagnose a large number of various diseases. Furthermore, in paragraph [0293] on page 22, right column of US 2008/0020379 A1, reference is merely made to customary statistical methods.

On page 24 in paragraph [0324], US 2008/0020379 A1 lists possibilities in diagnostics and in so doing refers to inflammatory diseases, wherein 48 inflammation genes for rheumatoid arthritis were used that come from a commercial source, i.e. “Source Precision Medicine”. Paragraph [0608] of page 45, right column, to page 46, left column, first section, quotes a list of genes conducted as “batch search” in the “Genetic Association database”.

Paragraph [0533] on page 44, left column, discloses that the biomolecular pathways that are expressed differentially at cell level are able to differentiate between adenovirus infections and non-adenovirus infections. To determine these pathways reference was made to the analysis following in paragraph [0533] by way of the KEGG pathway and the “Genetic Association” databases using EASE (70) to elucidate the functions of these genes with regard to molecular issues.

The aforementioned list of genes indicated in paragraph [0608] likewise belongs to this and US 2008/0020379 A1 thus relates to a distinction between adenovirus infections and non-adanovirus infections.

Nowhere in the description of US 2008/0020379 A1 are the test persons classified into local and systemic, depending on their status of infection and/or information.

Document US 2009/0307181 A1 relates to genetic analyses and the determination of genetic health scores for specific phenotypes such as, for example, diseases, disorders, treatments and conditions both for organ systems and for specific medical specialties and the overall state of health.

Paragraph [0195] of US 2009/0307181 A1 mentions, thereby referring to FIGS. 15 to 24, 26 to 33 and 39, that so-called panels of phenotype groups can be scrutinized within the scope of the document. The indicated panels are held generally and relate—without individual evidence—more or less to the entire clinical diagnostics and e.g. comprise such different inflammatory diseases as gastrointestinal diseases of unclear etiology, viral hepatitis, rheumatoid arthritis, systemic Lupus erythematosus, malaria, chronically obstructive lung diseases, autoimmune diseases as well as an infection panel (page 42, right column). In FIG. 15R of US 2009/0307181 A1, for example, rheumatoid arthritits is treated with a list of genes indicated in column 2 and so-called “reflex testing phenotypes”. However, now division into systemic or non-systemic was performed here, but the risk of falling ill with rheumatoid arthritis is scrutinized in connection with an exposure to the smoke of cigarettes.

FIG. 15V of US 2009/0307181 A1 includes Morbus Crohn as inflammatory bowel disease and/or ulcerative colitis. Moreover, as phenotypes, age and onset of the Crohn disease and localization and/or severity of the colitis are indicated therein. Paragraph makes reference to a set of phenotypes that, according to US 2009/0307181 A1, can be identified for a correlation of infectious diseases and pulmonology. Such phenotypes may include two or several phenotypes. Here, however, reference is merely made to general panels such as, for example, the World Infectious Disease Panel, HIV Panel, Malaria Panel, Viral Hepatitis Panel, Infection Panel, etc.

Paragraph [0408] on page 94, left column, of US 2009/0307181 A1, among numerous other possibilities, e.g. also indicates acute and chronic infections, sepsis and SIRS in addition to atrial fibrillation.

To a skilled person, the abundance of examples indicated in US 2009/0307181 A1, which, for the most part, are provided without biostatistical data, lack a reproducible teaching. This view is substantiated, for example, by feature b) of claim 1 of US 2009/0307181 A1, which reads “using a computer to determine the predisposition or carrier status of said individual for at least two phenotypes . . . .” Since no algorithms are referred to as to how such determination is to be performed, the teaching of US 2009/0307181 A1—as far as it is to be discerned at all—is inexplicable and not executable.

Document US 2010/0293130 A1 relates to genetic analysis systems and methods for these systems. According to the abstract the document essentially is about providing methods of determining a genetic composite index score for assessing an association between an individual's genotype and at least one disease or condition.

In particular, US 2010/0293130 A1 compares an individual's genomic profile with a database of medically relevant genetic variations that was established to be associated with one specific disease or one specific pathophysiological condition.

Document US 2010/0293130 A1, in paragraph [0115] on page 12, right column, discloses that a specific phenotype may be associated with corresponding genotypes correlated therewith. According to US 2010/0293130 A1, this may include Morbus Crohn, Lupus, Psoriasis as well as rheumatoid arthritis as inflammatory diseases.

Claim 1 of US 2010/0293130 A1 relates to a general method of generating at least one genetic composite index score based on a phenotype correlation without an explicit indication which genes are to be used. For lupus and rheumatoid arthritis in addition to a large number of other diseases it is to be seen from claim 133 on page 33 that a specific gene expression profile is generated and compared to a correlation between an SNP and a phenotype, and that a list of specific SNPs associating with a specific phenotype, is indicated.

Boldrick et al. (2002): Stereotyped and specific gene expression programs in human innate immune responses to bacteria, PNAS 99, 972-977 describes in particular on page 973, left column, last section, the host response to an immunological provocation with gram-negative bacteria, analyzed based on a group of 206 genes, however, nowhere in Boldrick et al. (2002) is a phenotype classification into local and systemic to be found.

Tang et al. (2007b): The Use of Gene Expression Profiling to Identify Candidate Genes in Human Sepsis, Am J Respir Crit. Care Med 176, 676-684 relates to the use of gene expression profiles for identifying gene candidates in human sepsis.

According to the abstract and the box indicated on page 676, right column, Tang et al. (2007b) relates to the diagnosis of sepsis by way of gene expression profiles and also mentions a mechanistic-biological insight into the host response in sepsis.

Tang et al. (2007b) thus relates to the “classic” approach of searching for specific “lead genes” for sepsis and correlating the gene expression thereof with a prognosis on the course of sepsis.

This is to be seen from the fact that a set of 50 signature genes, according to Tang et al. (2007b) correctly identified sepsis, with a prognosis probability of 91% and 88% in the training and validation sets. Tang et al. (2007b) further argue that specific genes that play a role in immunomodulation and inflammatory response showed a reduced expression in sepsis patients.

In particular, Tang et al. (2007b) demonstrate that activation of the core factor Kappa B metabolic pathway was diminished, whereas the corresponding inhibitor gene NFKBIA was controlled significantly high.

Accordingly, Tang et al. (2007b) concluded that the found signature genes suppress a suppression of the immune and inflammatory function of neutrophils in sepsis. In the view of authors Tang et al. (2007b) gene expression profiles thus offer a new approach in order to comprehend the host response in sepsis.

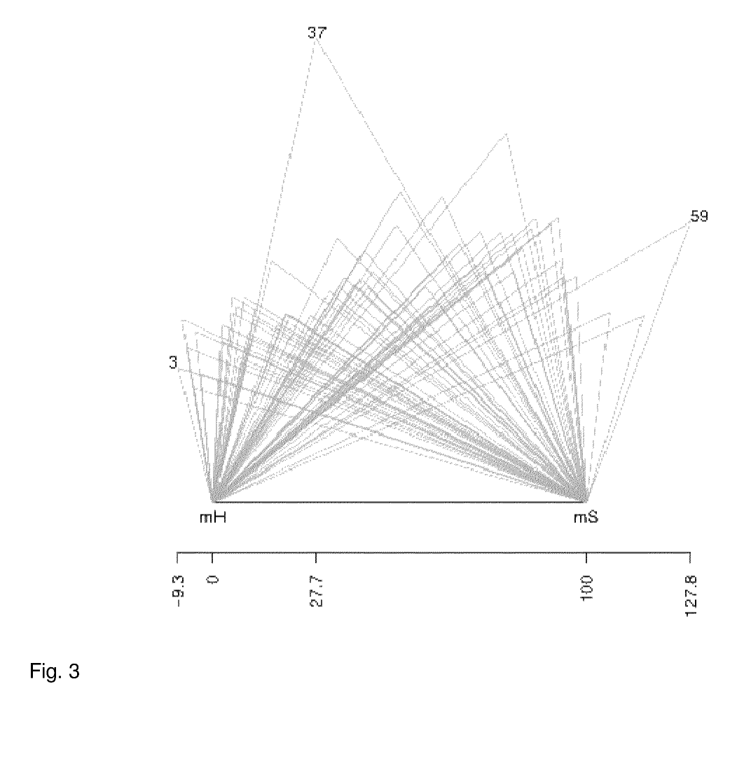

According to page 678 of Tang et al. (2007b) it is set forth under the key word “Statistical Analysis” that the authors developed a model for the prognosis of sepsis, thereby using the data of the training set. According to page 679, left column, section under the table, as well as FIG. 3A, Tang et al. (2007b) identified three clusters of coordinately expressed genes. According to the heat map of FIG. 3A, these clusters relate to a mitochondrial functional cluster, an immune regulation cluster as well as an inflammatory response cluster.

However, nowhere in Tang et al. (2007b) it is discernible that phenotypes are to be formed in accordance with claim 1 of the present application.

Warren et al. (2009): A Genomic Score Prognostic of Outcome in Trauma Patients, Mol Med 15, 220-227, relates to a genomic score that is to be prognostic to the outcome in trauma patients.

Finally, Xu et al. (2010) describe: Human transcriptome array for high-throughput clinical studies, PNAS 108, 3707-3712, a transcriptome array for high-throughput in clinical studies and in particular describes oligonucleotide arrays with 6.9 million oligonucleotides.

The present invention may be delimited against the prior art discussed at the beginning. The subject matter of the invention is the determination and follow-up observation of a reaction of the body to infectious and/or inflammatory stimulation, also referred to as host response, and a resulting “immune burden”. It is independent of the presence of a septic shock and is not restricted to such a group of patients. The present invention was made for determining a specific condition and not for distinguishing between survival vs. non-survival after a septic shock. Moreover, the present invention is independent of the presence of an infection in accordance with the current definition of sepsis. As will be shown in the present description, a critical condition, i.e. a maximum “immune burden” may exist even without an infection, e.g. an excessive stimulation of the innate system through other causes. The application of the invention consists in deriving suitable therapeutic measures and monitoring the follow-up of the disease, but not in predicting which of the patients will survive.

In a Review [Monneret et al., 2008] the importance of an effectively functioning immune system is illustrated based on a number of scientific results, and it is summarized which trials render such hypothesis plausible. At the same time the review sets forth that suitable methods for routine determination of an immune state still have to be determined.

The prior art includes numerous studies for identifying gene expression markers [Tang et al., 2007b] or gene expression profiles for determining a systemic infection [Johnson et al., 2007].

Tang and colleagues [Tang et al., 2007b] looked in a particular blood cell population, the neutrophils, for a signature which makes it possible to distinguish between patients with SIRS and sepsis. 50 markers from this cell population suffice to reproduce an immune response to systemic infection and enable new discoveries into the pathophysiology and the involved signaling pathways.

The classification of patients with and without sepsis succeeds with high reliability (PPV 88% and 91% in training and test data sets). The applicability for clinical diagnosis is, however, limited by the fact that in blood the signature of signals from other blood cell types can be overlaid. Regarding the applicability, the preparation of such a blood cell population is associated with a significantly increased effort. The significance of the results published in this study, however, is limited for practical applications because the patient selection was very heterogenous. Patients were included in the study that had very different concomitant diseases such as e.g. up to 11% to 16% tumor diseases, or were subjected to very different therapeutic measures (e.g. 27% to 64% vasopressor therapy), whereby gene expression profiles were strongly affected.

Johnson and colleagues [Johnson et al., 2007] describe on the basis of a group of trauma patients that the expression of sepsis can be measured based on molecular alterations already up to 48 hours prior to clinical diagnosis. The trauma patients were examined over several days. Part of the patients developed sepsis. Noninfectious SIRS patients were compared to pre-septic patients. The identified signature of 459 transcripts consisted of markers of the immune response and inflammatory markers. The sample was whole blood, the analyses were performed on a microarray. It is unclear as to whether this signature can be expanded also to other types of groups of septic or pre-septic patients. A classification and diagnostic benefit of this signature was not shown.

The objective of all those papers is to identify an infectious occurrence by differential gene expression. Thus, those publications can be delimited well from the subject matter of the present invention, i.e. the identification and follow-up observation of a reaction of the body to infectious and/or inflammatory stimulation, also referred to as host response, and a resulting “immune burden”.

The goal of Feezor and colleagues [Feezor et al., 2003] was to identify differences between infections with gram-negative and gram-positive pathogens. Blood samples of three different donors were stimulated ex vivo with E. coli-LPS and heat-activated S. aureus. Using microarray technology, gene expression studies were performed. The work group found both genes that were up-regulated after S. aureus stimulation and down-regulated after LPS stimulation, and that were more strongly expressed after treatment with LPS than after the addition of heat-activated S. aureus germs. At the same time, many genes were upregulated to the same extent by gram-positive and gram-negative stimulation. This relates, for example, to cytokines TNF-α, IL-1β and IL-6. The differentially expressed genes unfortunately were not published by name, so that only an indirect comparison with other results is possible. In addition to the gene expression, Feezor and colleagues also examined the plasma concentrations of some cytokines. It was found that the gene expression data did not necessarily correlate with the plasma concentrations. In gene expression, the quantity of mRNA is measured. This, however, is subject to posttranscriptional regulation prior to protein synthesis, from which the observed differences may have resulted.

The most interesting publication on this subject was published by the Texan research group of Ramilo [Ramilo et al., 2007]. Here, gene expression studies on human blood cells also were carried out, which uncovered differences in the molecular host response to various pathogens. For this, pediatric patients with acute infections such as acute respiratory diseases, urinary tract infections, bacterimea, local abscesses, bone and joint infections and meningitis were examined. Microarray experiments were carried out with RNA samples which were isolated from peripheral mononuclear blood cells of ten patients respectively, with E. coli or S. aureus infection. Identification of the pathogens was carried out by blood culture. On the basis of the training data set 30 genes were identified by the use of which the causative pathogen germs could be diagnosed with high accuracy.

Those papers can be delimited clearly from the present invention as herein, the host response of the causative pathogens is to be identified by way of gene expression signatures, whereas the invention is to be used for determination and follow-up observation of the reaction of a body to infectious and/or inflammatory stimulation and a resulting “immune burden”.

None of those publications offers the reliability, accuracy and robustness of the invention disclosed here. These studies are focused on identifying the, from a scientific perspective, “best” multi-gene biomarker (classifier), but not, as in the present invention, the optimal multi-gene biomarker for a specific clinical problem [Simon et al., 2005].

Thus, it is the object of the present invention to provide a test system with which rapid and reliable assessment of a pathophysiological condition, in the present case the determination and follow-up observation of the reaction of a body to infectious and/or inflammatory stimulation, also referred to as host response, and the resulting “immune burden”, can be made without having to rely on condition-specific biomarkers.

The invention relates in particular to a method for identifying a subset of polynucleotides from an initial set of polynucleotides corresponding to the human genome for the in vitro determination of the severity of the host response of a patient being in an acutely infectious and/or acutely inflammatory condition, in a sample, a measuring device being used that comprises a plurality of different gene probes that essentially represent the entire human genome, wherein
samples of nucleic acid of a plurality of test persons exhibiting a known phenotypic physiological condition are brought into contact with the probes of the measuring device so as to obtain signals of the respective expression of a gene;
of the total number of gene probes deployed, those are selected that provide an expression signal of detectable intensity for at least one sample of nucleic acid of a test person;
the test persons, depending on their infectious and/or inflammatory status, are divided into at least two of the following clinically determined groups of phenotypes:

Inflammation

| | Systemic | Local | None |
|---|---|---|---|
| Infection | [S] | [L] | [N] |
| Systemic [S] | SaS | | |
| Local [L] | LaS | LaL | |
| None [N] | NaS | NaL | NaN |

wherein a“ represents an AND-operation between the properties S, L and N;

the changes of the gene expression signals between the groups of phenotypes are compared statistically and it is assessed as to whether there is a significant difference between at least two of the groups of phenotypes;
those gene probes are selected the gene expression signals of which have significantly changed statistically between at least two groups of phenotypes and an estimated number of those gene probes is excluded that provide a false positive result in relation to a predetermined threshold value;
a master score is determined as measurement for the severity of the host response of a test person being in an acutely infectious and/or acutely inflammatory condition, by quantifying an increase and decrease in the gene expression intensity of the selected gene probes; and

compared to the initial set, a considerably reduced number of polynucleotides is identified by determining a score that comprises at most a predetermined deviation from the master score and that likewise serves as a measurement for the severity of the host response of a test person being in an acutely infectious and/or acutely inflammatory condition.

Moreover, the present invention relates to the method of use of k-tuples of polynucleotides selected from the group consisting of m polynucleotides with SEQ ID No: 1 to SEQ ID NO: 7704, wherein k is at least 7 and equal to or lower than the number of polynucleotides m in the group; for identifying a score as measurement for the severity of the host response of a test person being in an acutely infectious and/or acutely inflammatory condition.

The method of use of k tuples of polynucleotides selected from a group consisting of m poly-nucleotides with SEQ ID No: 1 to SEQ ID No: 7704, wherein k is at least 7 and equal to or lower than the number of polynucleotides m in the group for performing the method in accordance with the invention.

The sub-claims relate to preferred embodiments of the present invention.

In Applicant's practice it has turned out that such a method of use is particularly suited that is characterized by the gene activities being captured by way of enzymatic methods, particularly amplification methods, preferably polymerase chain reaction (PCR), preferably real time PCR, especially probe-based methods such as Taq-Man, Scorpions, Molecular Beacons; and/or by way of hybridization methods, particularly those on micro arrays; and/or direct proof of mRNA, particularly sequencing or mass spectrometry; and/or isothermal amplification [Valasek et al., 2005; Klein 2002]. Those classical methods allow for proving in a highly sensitive manner DNA and, via reverse transcription (RT), also RNA [Wong et al., 2005; Bustin, 2002].

Real time PCR, also referred to as quantitative PCR (qPCR), is a method for detecting and quantifying real time nucleic acids [Nolan et al., 2006]. In molecular biology, it has already been part of established standard techniques for several years.

The quantitative determination of a template may be done by way of absolute or relative quantification. In absolute quantification, the measurement is done based on external standards, e.g. plasmid DNA in various dilutions. In contrast thereto, relative quantification makes use of so-called housekeeping or reference genes as reference [Huggett et al., 2005].

For the methods in accordance with the invention (array technique and/or amplification methods) the sample is selected from tissue, body fluids, particularly blood, serum, plasma, urine, saliva or cells or cell components, or a mixture thereof.

It is preferred that samples, particularly samples of cells, are subjected to lytic treatment so as to liberate the cell contents thereof.

It is clear to a skilled person that the individual features of the invention set forth in the claims may be combined with each other arbitrarily without restriction.

Further advantages and features of the present invention are revealed by the description of embodiments as well as the drawing.

A further preferred embodiment of the present invention lies in an application that is characterized by an index being formed of the individual specific gene activities, which index, after corresponding calibration, constitutes a measurement for the degree of severity and/or the process of a pathophysiological condition, wherein the index preferably is shown on a scale that can be interpreted easily.

Furthermore, it is preferred that the gathered gene activity are used for creating software for describing at least one pathophysiological condition and/or examination question and/or adjuvant for diagnostic purposes and/or management systems for the data of patients, particularly for the use of patient stratification and as inclusion criterion for clinical studies.

Moreover, an application is preferred in which for compiling gene activity data, specific gene loci, sense and/or antisense strands of pre-mRNA and/or mRNA, small RNA, particularly scRNA, snoRNA, micro RNA, siRNA, ncRNA, or transposable elements, genes and/or gene fragments with a length of at least 5 nucleotides are used that have a sequence homology of at least approximately 10%, in particular approximately 20%, preferably approximately 50%, and a particularly preferred sequence homology of about 80% with regard to the polynucleotide sequences in accordance with SEQ ID No: 1 to SEQ ID No: 7704.

Preferably, the sample nucleic acid is RNA, in particular whole RNA or mRNA, or DNA, in particular cDNA.

However, it has to be emphasized that the above-indicated primers are merely examples.

The above-indicated amplicons may be used, for example, as probes for hybridization methods.

Within the scope of optimized EDP-supported hospital management and for further research in the field of sepsis it has turned out to be advantageous that the gene activity data gathered are utilized for creating software for the description of at least one pathophysiological condition and/or an examination question and/or as adjuvant for diagnostic purposes and/or for management systems for the data of patients.

What is preferred is a multi-gene biomarker, a combination of several poly-nucleotide sequences, particularly gene sequences, based on the gene activities of which a classification may be performed by way of an interpretation function and/or an index or score formed.

For the purpose of the present invention it has turned out to be advantageous that the gene activities are detected by way of enzymatic methods, especially amplification methods, preferably polymerase chain reaction (PCR), preferably real time PCR; and/or by way of hybridization methods, particularly those on microarrays.

Differential expression signals of polynucleotide sequences contained in the multi-gene biomarker occurring during the detection of the gene activities, can be associated with a pathophysiological condition, process and/or therapy monitoring in an advantageous and clear manner.

The score can put a rapid diagnostic tool in the hands of the doctor in charge.

Applicant has developed several methods that make use of different sequence pools in order to ascertain and/or differentiate conditions or respond to defined research issues. Examples for this are to be found in the following printed patent specifications: differentiating between SIRS, sepsis and sepsis-like conditions [WO 2004/087949; WO 2005/083115], establishing criteria for predicting the disease course in sepsis [WO 05/106020], differentiating between non-infectious and infectious causes of multiple organ failure [WO 2006/042581], in vitro classification of gene expression profiles of patients with infectious/non-infectious multiple organ failure [WO 2006/100203], establishing the local causes of fever of unclear origin [WO 2007/144105], polynucleotides for detecting gene activities for distinguishing between local and systemic infections [DE 10 2007 036 678.9].

The invention relates to polynucleotide sequences, a method and also kits for creating multi-gene biomarkers that in one and/or several modules exhibit features of an “In Vitro Diagnostic Multivariate Index Assay” (IVDMIA) [FDA: In Vitro Diagnostic Multivariate Index Assays, 2007].

With regard to the nucleotide sequences used in the present application the following is to be noted:

RefSeq is a public database which includes information of nucleotide and protein sequences with their properties as well as bibliographic information.

The RefSeq database was established by the National Center for Biotechnology Information (NCBI), a division of the National Library of Medicine which is part of the US National Institute of Health, and is maintained and updated continuously [Pruitt et al., 2007].

The NCBI creates RefSeq from the sequence data of the archive database “GenBank” [Benson et al., 2009], a comprehensive public database of sequences set up in GenBank in the U.S.A, the EMBL data library in the United Kingdom, and the DNA database of Japan and also data exchanged between these databases.

The RefSeq collection is unique with regard to the provision of error-corrected, non-redundant, explicitly linked nucleotide and protein databases. The entries are non-redundant with the aim to represent the different biological molecules that are characteristic to an organism, strain or haplotype.

If certain entries in the collection occur multiple times, there may be several reasons for this:
alternative spliced transcripts encode for the same protein product (so-called transcript variants),
there are several genomic areas within a species or between species which encode for the same protein product,
when RefSeqs are created, which represent alternative haplotypes, and some of mRNA and protein sequences are identical in all haplotypes.

The RefSeq database provides the critical foundation for sequence integration, genetic and functional information and is regarded internationally as the standard for genome annotation. In a sequence search using BLAST, the RefSeq indications are available in several NCBI resources including Entrez Nucleotide, Entrez Protein, Entrez Gene, Map Viewer, the FTP download, or by networking with PubMed [Pruitt et al., 2007; The NCBI handbook 2002]. RefSeq information may be identified by the unique accession format including an underscore (_).

Workgroups make use of various methods and listings and compile the RefSeq collection for different organisms. RefSeq records are created by using several different methods [The NCBI handbook 2002]:
1. Scientific cooperation
2. Computer-assisted genome annotation processes
3. Error-correction by the NCBI staff
4. Extracts from GenBank

Each item of data is provided with a comment indicating the status of the corresponding error correction as well as the allocation to the cooperating workgroup. Thereby the RefSeq indication either contains an essentially unchanged, initially valid copy of the original GenBank entries, or corrected and additional information added by cooperation partners or experts [The NCBI handbook 2002].

If a molecule in GenBank is represented by several sequences, the NCBI staff makes the decision for the “best” sequence, which is then presented as RefSeq.

The decision to use the marker population named in the present application on the basis of its RefSeq identity for the purpose of the present invention was made on account of the above-described properties of the RefSeq database. The characteristic features of the database concerning the preparation, quality, care and updates on biological sequences, as well as the existence of functional information on the nucleic acid level, equally for alternative splice variants, were the decisive factor.

As was already explained, the biological mechanism of alternative splicing offers flexibility to a skilled person to extend the scope of protection. Thus, it is conceivable that with new transcript variants completely new primary structures will be identified, or that sequence changes will occur in the known transcript variants. On the other hand, those genomic regions are claimed that encompass for all these known and unknown variants of coding transcripts, including their cis-regulatory sequences as complete genomic functional units and thus fall within the scope of the present invention, or at least make available to the person of ordinary skill in the art easily obtainable equivalents to those sequences indicated in the claims, the description and sequence listing.

### DEFINITIONS

For the purpose of the present invention the following definitions are used:

SIRS: Systemic Inflammatory Response Syndrome, according to Bone [Bone et al., 1992] and Levy [Levy et al., 2003], a generalized, inflammatory, non-infectious condition of a patient.

Sepsis: according to Bone [Bone et al., 1992] and Levy [Levy et al., 2003], a generalized, inflammatory, infectious condition of a patient.

Inflammation: this is a reaction of the body caused by injury or destruction of tissue that is intended to remove, dilute or isolate the injuring agent or injured tissue.

An inflammatory process may be caused by physical, chemical or biological agents, including mechanical traumas, exposure to radiation by the sun, Roentgen rays and radioactive radiation, corrosive chemicals, extreme heat or cold and infectious agents such as bacteria, viruses, fungi, and other pathogen organisms. However, the terms inflammation and infection cannot be used as synonyms.

Classical indices of an inflammation are heat, redness, swelling, pain and loss of function of the tissue concerned. These are manifestations of physiological changes that occur during an inflammatory process. The three main components of such a process are:
1) Changes in the diameter of blood vessels and rate of the blood flow through these vessels (hemodynamic changes)
2) Enhanced permeability of the capillaries, and
3) Wandering of leukocytes

Infection: the penetration of pathogen microorganisms into the body and their multiplying therein, which cause a disease through injury of cells or cell complexes, secretion of toxins, or through antigen-antibody reaction of the host.

A systemic infection is an infection in which the pathogens have spread via the bloodstream throughout the body.

Biological fluid: biological fluid, within the context of the invention, refers to all body fluids of mammals, including humans.

Gene: a gene is a segment of desoxyriobonucleic acid (DNA), which contains the basic information for making a biologically active ribonucleic acid (RNA) as well as regulatory elements that activate or inactivate such manufacture. As genes within the context of the invention, also all derived DNA sequences, partial sequences and synthetic analogs (e.g. peptidonucleic acids (PNA)) are understood. The description of the invention being related to determining gene expression on an RNA level thus expressly does not constitute a restriction, but merely an exemplary application.

Gene locus: Gene locus is the position of a gene in the genome. If the genome consists of several chromosomes, the position within the chromosome is meant on which the gene is located. Different forms or variants of this gene are referred to as alleles, which are all located in the same location on the chromosome, i.e. the gene locus. Thus, the term “gene locus” on the one hand includes the pure genetic information for a specific gene product and on the other hand all regulatory DNA segments as well as all additional DNA sequences which are related to the gene on the gene locus in any functional relationship. The latter attach to sequence regions that are located in the immediate vicinity (1 Kb), but outside of the 5- and/or 3′-end of a gene locus. The specification of the gene locus is done by the accession number and/or RefSeq ID of the RNA main product which is derived from the locus.

Gene activity: By gene activity, the ability of a gene to be transcribed and/or to form translation products is comprehended.

Gene expression: The process of forming a gene product and/or expression of a genotype into a phenotype.

Multi-gene biomarker: A combination of several gene sequences, the gene activities of which produce a combined overall result (e.g. a classification and/or index), using an interpretation function. This result is specific to one condition and/or a research issue.

Hybridization conditions: The physical and chemical parameters well known to a skilled person, which may influence the establishment of a thermodynamic equilibrium of free and bound molecules. In the interest of optimal hybridization conditions the duration of contact of the probe and sample molecules, the cation concentration in the hybridization buffer, temperature, volume as well as concentrations and concentration ratios of the hybridizing molecules must be coordinated.

Amplification conditions: Constant or cyclically changing reaction conditions which allow for the multiplication of the base material in the form of nucleic acids. The reaction mixture includes the individual components (desoxyribonucleotides) for the resulting nucleic acids, as well as short oligonucleotides which may attach to complementary areas in the base material, and a nucleic acid syntheses enzyme referred to as polymerase. Cation concentrations, pH-value, volume and duration and temperature of individual reaction steps that a skilled person is well aware of, are of importance in the progress of amplification.

Primer: In the present invention, an oligonucleotide is referred to as primer, which serves as starting point for nucleic acid replicating enzymes such as, for example, DNA polymerase. Primers may consist both of DNA and RNA (Primer3, confer e.g. http://frodo.wi.mit.edu/cgi-bin/primer3 www.cgi of the MIT).

Probe: In the present application, a probe is a nucleic acid fragment (DNA or RNA) that may be provided with a molecular marker (e.g. fluorescent label, especially Scorpion®, molecular beacons, Minor Groove Binding probes, TaqMan® probes, isotope marking, etc.) and is used for sequence-specific detection of target DNA and/or target RNA molecules.

PCR: is the abbreviation for the English term “Polymerase Chain Reaction” (PCR). The polymerase chain reaction is a method for reproducing DNA in vitro outside a living organism with the aid of a DNA-dependent DNA polymerase. In accordance with the present invention PCR is used in particular to reproduce short segments—of up to about 3,000 base pairs—of a DNA strand of interest. This may be a gene or only part of a gene or even non-coding DNA sequences. The skilled person is well aware of the fact that a number of PCR methods are known in the art, all of which are encompassed by the term “PCR”. This is particularly true for the “Real-Time PCR” (also cf. the explanations below).

Transcript: For the purpose of the present application, a transcript is to be understood as any RNA product that is manufactured based on a DNA template.

Small RNA: refers to small RNAs in general. Representatives of this group are in particular, but not exclusively:

a) scRNA (small cytoplasmatic RNA) which is one of several small RNA molecules in the cytoplasm of a eukaryote.

b) snRNA (small nuclear RNA), one of many small forms of RNA that occur only in the nucleus. Some of the snRNAs play a role in splicing or other RNA-processing reactions.

c) small non-protein-coding RNAs, which include the so-called small nucleolar RNAs (snoRNAs), microRNAs (miRNAs), short interfering RNAs (siRNAs) and small double-stranded RNAs (dsRNAs) involved in gene expression at many levels, including chromatin architecture, RNA editing, RNA stability, translation and possibly also transcription and splicing. In general, these RNAs are processed in multiple ways from the introns and exons of longer primary transcripts, including protein-coding transcripts. Although approximately only 1.2% of the human genome encodes proteins, a large portion is nevertheless transcribed. In fact, approximately 98% of the transcripts found in mammals and humans consist of non-protein-coding RNAs (ncRNA) from introns of protein-coding genes and the exons and introns of non-protein-coding genes, including many which are anti-sense to protein-coding genes or overlap with these. Small nucleolar RNAs (snoRNAs) regulate the sequence-specific modification of nucleotides in target RNAs. Herein, there are two types of modifications, i.e. 2′-O-ribose methylation and pseudouridylation, which are regulated by two large snoRNA families referred to, on the one hand, as box C/D-snoRNAs and, on the other hand, as box H/ACA snoRNAs. Such snoRNAs have a length of approximately 60 to 300 nucleotides. miRNAs (microRNAs) and siRNAs (short interfering RNAs) are even smaller RNAs with 21 to 25 nucleotides in general. miRNAs come from endogenous short hairpin precursor structures and usually use other loci with similar, but not identical sequences as the target of translational repression. siRNAs arise from longer double-stranded RNAs or long hairpins, often of exogenous origin. They usually have homologous sequences at the same locus or elsewhere in the genome as the target, where they are involved in the so-called gene silencing, a phenomenon also referred to as RNAi. The boundaries between miRNAs and siRNAs, however, are blurred.

d) In addition, the term “small RNA” also may include the so-called transposable elements (TEs), especially the retro elements, which likewise, for the purpose of the present invention, fall within the meaning of the term “small RNA”.

RefSeq ID: This term refers to the entries in the NCBI database (www.ncbi.nlm.nih.gov). The database provides non-redundant reference standards for genomic information. Such genomic information includes, inter alia, chromosomes, mRNAs, RNAs and proteins. Each RefSeq ID represents a single, naturally occurring molecule of an organism. The biological sequences, which represent a RefSeq, are derived from GenBank entries (also NCBI), but are a compilation of information elements. These information elements come from primary research on DNA, RNA and protein level.

Accession number: An accession number is the entry number of a polynucleotide in the NCBI GenBank database which is known to a skilled person. In this database, both RefSeq IDs and less well-characterized and redundant sequences are administered and made available to the public (www.ncbi.nlm.nih.gov/genbank/index.html).

Local infection: The infection is limited to the portal of entry of the pathogen (e.g. wound infection).

Generalized infection: Pathogens penetrate into the vascular system, thereby affecting the whole organism. Generalized infections may lead to sepsis.

Colonization: The presence of micro-organisms does not provoke any symptoms of a disease in the organism.

Bacteremia: A condition in which bacteria are present in the blood shortly and temporarily, without this necessarily being associated with the occurrence of bacterial clinical symptoms.

Alternative splicing: a process in which the exons of the primary gene transcript (pre-mRNA) are reconnected after excision of introns in various combinations.

BLAST: Basic Local Alignment Search Tool [according to Altschul et al., J Mol Biol 215: 403-410; 1990]. Sequence comparison algorithm, speed-optimized, is used for the search in sequence databases for optimal local conformity to the request sequence.

cDNA: Complementary DNA. DNA sequence, product of reverse transcription of mRNA.

Coding sequence: Protein-coding segment of a gene or an mRNA to distinguish it from introns (non-coding sequences) and 5′- or 3′-nontranslated segments. Coding sequences of cDNA or the mature mRNA include the area between the start (AUG or ATG) and stop codon.

EST: Expressed Sequence Tag. Short ssDNA segments of cDNA (typically ≈300-500 bp), usually produced in large quantities. Represent the genes that are expressed in particular tissues and/or during certain development phases. Partially coding or non-coding labels of expression for cDNA libraries. Valuable for determining the size of complete genes and in the context of mapping.

Exon: Coding sequence area of typical eukaryotic genes corresponding to mRNA. Exons may include the coding sequences, the 5′-nontranslated area, or the 3′-nontranslated area. Exons encode specific sections of the complete protein and are usually separated by long segments (introns) which sometimes are referred to as “junk DNA”, the function of which is not precisely known, but which probably encode short, nontranslated RNAs (snRNA) or regulatory information.

GenBank: Nucleotide sequence database with sequences from more than 100,000 organisms. Records that are annotated with properties of the coding areas, also include the translation products. GenBank is part of the international cooperation of sequence databases, which also includes EMBL and DDBJ.

Intron: Non-coding sequence area of a typical eukaryotic gene which is excised out of the primary transcript during RNA splicing and thus is no longer present in the mature, functional mRNA, rRNA or tRNA.

mRNA: Messenger RNA or sometimes only “message”. RNA which contains the sequences necessary for protein coding. The term mRNA is used, to distinguish it from the (unspliced) primary transcript, merely for the mature transcript with polyA-tail (exclusive of the introns removed by splicing). Has 5′-nontranslated, amino acid coding, 3′-nontranslated areas and (almost always) a poly(A)-tail. Typically constitutes about 2% of the total cellular RNA.

Poly(A)-tail: ss adenosine extension 50-200 monomers) which, during splicing, is hung to the 3′-end of the mRNA. The poly(A)-tail presumably increases the stability of the mRNA (possibly protection against nucleases). Not all mRNA have this construct, for example, the histone mRNA.

RefSeq: NCBI database of reference sequences. Error-corrected, non-redundant sequence collection of genomic DNA contigs, mRNA and protein sequences and sequences or of known genes and complete chromosomes.

SNPs: Single Nucleotide Polymorphisms. Genetic differences between alleles of the same gene based on single nucleotide deviations. Emerge at specific individual positions within a gene.

Transcript variants: Alternative splicing products. The exons of the primary gene transcript (pre-mRNA) were reconnected in different ways and are subsequently translated.

3′-non-translated region: Transcribed 3′-terminal mRNA area without protein-coding information (region between stop codon and poly(A)-tail). May influence the translation efficiency or stability of the mRNA.

5′-non-translated region: Transcribed 5′-terminal mRNA area without protein-coding information (area between initial 7-methylguanosine and the base immediately before the ATG start codon). May influence the translation efficiency or stability of the mRNA.

Polynucleotide isoforms: Polynucleotides with the same function, but different sequence.

### ABBREVIATIONS

CRP C-reactive Protein
OR Odd Ratio
PCT Procalcitonin
Sensitivity Proportion of correct tests in the group with specified disease (infectious SIRS or sepsis)
Specificity proportion of correct tests in the group without specified disease (non-infectious SIRS)

Moreover, non-prepublished DE 10 2009 044 085 discloses a system comprising the following elements:
A set of gene activity markers
Reference genes as internal control of gene activity marker signals in whole blood
Detection mainly via Real Time PCR or other amplification or hybridization methods
Use of an algorithm to convert the individual results of the gene activity markers into a common numeric value, index or score
Representation of this numeric value on a correspondingly scaled scale
Calibration, i.e. dividing up the scale according to the intended application by previous validation experiments.

The system provides a solution to the problem of determining disease conditions such as, for example, the distinction of infectious and non-infectious multiple organ failure, but also for other applications and problems relevant in this context.

All approaches for the diagnostic/prognostic detection of inflammatory and/or infectious conditions that were pre-published in the prior art and contained in the above-indicated document DE 10 2009 044 085 that had not yet been published at the filing date of the present application, however, have, as was set forth at the beginning, found access to clinical routine merely with restraint.

A trial for differential gene expression from peripheral samples of whole blood was carried out, thereby using a broad spectrum of inflammatory-infectious clinical phenotypes, ranging from the healthy test person over patients with local inflammation and local infection to intensive care patients with systemic inflammation (SIRS) and systemic infection (sepsis), and a measuring platform representing the total human genome in the form of 25,000 different probes. As a result, surprisingly a transcriptomic signature was identified that, instead of erratic changes based on the different phenotypes, represents an inflammatory-infectious continuum of differential gene expression. A further surprising result that followed from this finding was the lack of infection-specific gene groups. Moreover, in the group with systemic inflammation a differential gene expression could be ascertained that was comparable to that of patients with infections in the blood stream.

These results may provide an explanation to the finding explained in the following that the detection of a pathogen from blood samples succeeds in less than half of the cases under suspicion of sepsis. A condition that is represented by wide, simultaneous over- and under-expression of specific gene transcripts, may be regarded as critical and may include features of sepsis indicated in the subsequent sections. It is characterized by a reaction of the body to infectious and/or inflammatory stimulation and a resulting “immune burden”, also referred to as host response. The information on such a condition, which is represented by all significant differentially expressed gene transcripts, may be summarized mathematically to a non-dimensional numeric value, a score, and can be depicted as an interval to the condition of a healthy person. The larger the numeric value of the score, the larger is the intensity of a reaction of the body to infectious and/or inflammatory stimulation and the resulting “immune burden”. Comparable information on the reaction of the body to infectious and/or inflammatory stimulation and the resulting “immune burden” likewise may be gathered on the basis of scores, formed of sub-selection from all differentially expressed genes.

The reaction of the body to infectious and/or inflammatory stimulation and the resulting “immune burden” cannot only become stronger or deteriorate, but also may move in the opposite direction, i.e. move towards the condition of a healthy person or improve. This reversion may be regarded as recovery process. If this recovery follows directly on a therapeutic measure, it is an indication of success of the therapy.

Progression of the condition into a critical area is indicative of a patient's high risk of mortality. In such a condition there is a high probability for the patient to suffer a life-threatening complication in the form of an uncontrolled primary infection and/or secondary infection and/or die of the consequences of an uncontrolled inflammatory-infectious host response.

The progression towards a critical condition may be utilized as early diagnosis based on which the required therapeutic measures and interventions are initiated.

The reaction of the body to infectious and/or inflammatory stimulation and the resulting “immune burden” may be used as relevant specification for the detection of a condition.

The immune status may be used by means of timely succeeding multiple determinations for follow-up monitoring and therapy control in patients.

Medical measures may be medicinal treatment and its escalation or de-escalation, invasive measures such as surgical operations for infectious source control and/or further diagnostic measures.

The determination of the immune status may be used for differential diagnosis in that it is ascertained whether the immune system contributes to the acute incidence of the disease, or is ineligible as cause of the life-threatening condition of a patient.

The objective of most gene expression studies for sepsis diagnosis so far was to find markers that distinguish as to whether or not a systemic inflammatory response syndrome (SIRS, ACCP/SCCM, 1992) was caused by pathogens. In these studies, especially samples of intensive care patients were analyzed. Distinction into the groups “sterile SIRS” vs. “sepsis” (SIRS and positive detection of a pathogen) was done particularly in accordance with the microbiological proof. The studies provided very heterogeneous results, as was ascertained in a current comparative publication [Tang et al. (2010)]. With its experimental approach, the present invention examines the causes for this. The trial scheme of the inventors was based on the following concept:

The previous approach for diagnosing sepsis was gathered from the modus operandi in case of a local inflammation. Here, too, one wants to distinguish as to whether or not the inflammation on an organ was caused by a pathogen (this is e.g. referred to as bacterial or sterile myocarditis or pancreatitis). Infection-specific gene markers must exhibit an increased or else reduced expression even in an infection, which manifests itself not necessarily by a systemic inflammation, but merely causes an inflammation in the organ/locus affected. However, this could not be shown in case of the gene markers found so far in the prior art.

Another explanation for heterogeneous results would be that the gene expression studies make use of RNA samples from blood. Thus, the best chances of finding infection-specific gene markers should be provided if merely samples with a positive blood culture, which is a “gold standard” for infections in the blood stream, are measured. The previous studies viewed sepsis patients as one study group, independent of a spread of the infection.

Applicant bases its studies on a trial scheme in which the features of infection and inflammation in their spatial proliferation are classified independent of one another. A distinction was made as to whether the inflammation was systemic, local or not distinctive at all. In doing so, systemic inflammation was determined by the definition of the systemic inflammatory response syndrome (SIRS). Moreover, it was examined whether pathogens were found in the blood (systemic), on an organ (local), or not at all. The 9 possible phenotypes were defined from a combination of the proliferation of infection and inflammation. They are summarized in table 1.

TABLE 1
Representation of the phenotypes that result from the spatial
manifestation of an inflammation and/or infection. Each phenotype
is identified by a token of 3 letters. The first capital letter
refers to an escalation of the infection, the second letter
relates to an escalation of the inflammation. Both letters
were connected by an “a” (standing for and).

Inflammation

| | Systemic | Local | None |
|---|---|---|---|
| Infection | [S] | [L] | [N] |
| Systemic [S] | SaS | SaL | SaN |
| Local [L] | LaS | LaL | LaN |
| None [N] | NaS | NaL | NaN |

In the view of the present invention, such a division is clear, complete and independent of other factors, i.e any test person may be associated to one of the groups at the time of taking the samples.

While an inflammation is not necessarily caused by pathogens, an infection without an inflammatory reaction is not relevant diagnostically. A systemic infection that merely causes a localized inflammation (so-called bacteremia, e.g. in case of an endocarditis) is a rare phenomenon and constitutes a special case. Therefore, the combinations LaN, SaN and SaL do not form clinically relevant phenotypes and were disregarded for the purpose of the present invention.

For their study, the inventors divided diagnostically relevant samples of patients according to the spatial proliferation of an inflammation and/or infection, which resulted in 6 study groups (indicated in bold letters in table 1). These groups represent the most important and most frequent infection-inflammatory phenotypes. Particularly the 4 phenotypes with a local infection with local and systemic inflammation (LaL and LaS) as well as the corresponding control groups without an infection (NaL and NaS) make, in a statistical comparison, the discovery of infection-specific gene markers possible. A comparison with groups SaS and LaS provides hints as to whether in systemic inflammation the infection of the circulating cells (blood stream infection) is indicative of a different gene expression pattern than in case of a locally restricted infection.

A sequence listing with SEQ-ID-numbers 1-7718 is attached to the present application, the contents of which listing fully is part of the disclosure content of the present application.

Further advantages and features are to be seen from the description of embodiments as well as the drawing.

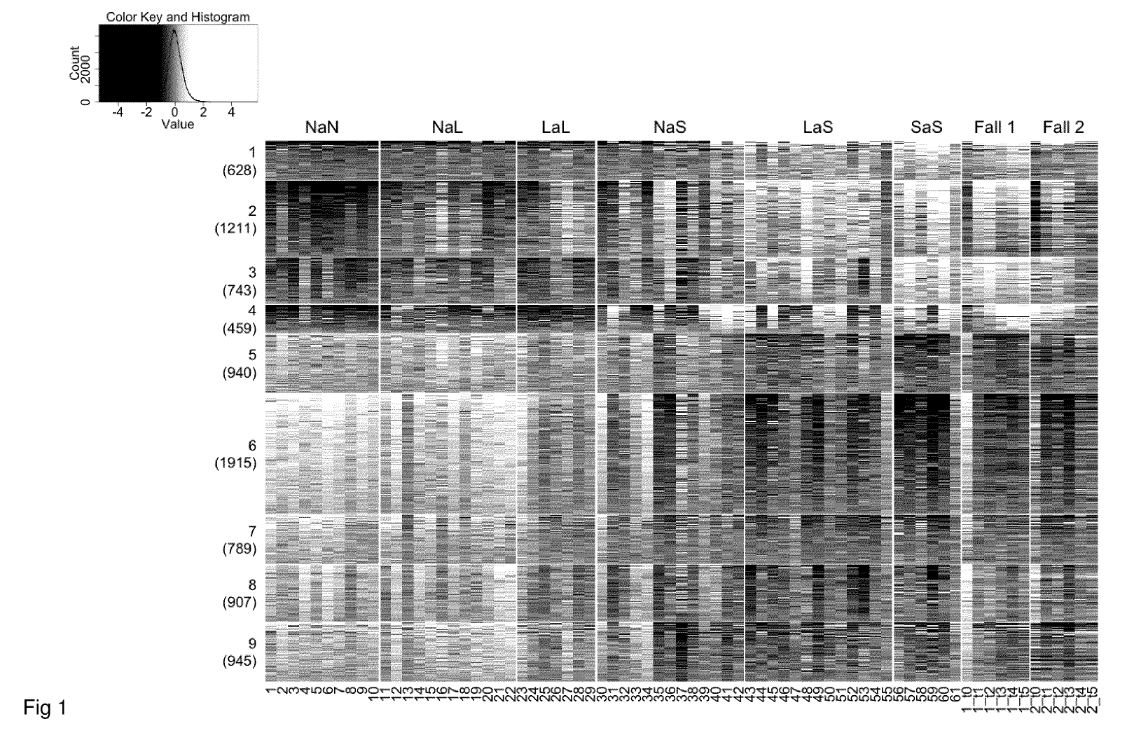

FIG. 1 shows a heat map in which the expression patterns are sorted by study groups;

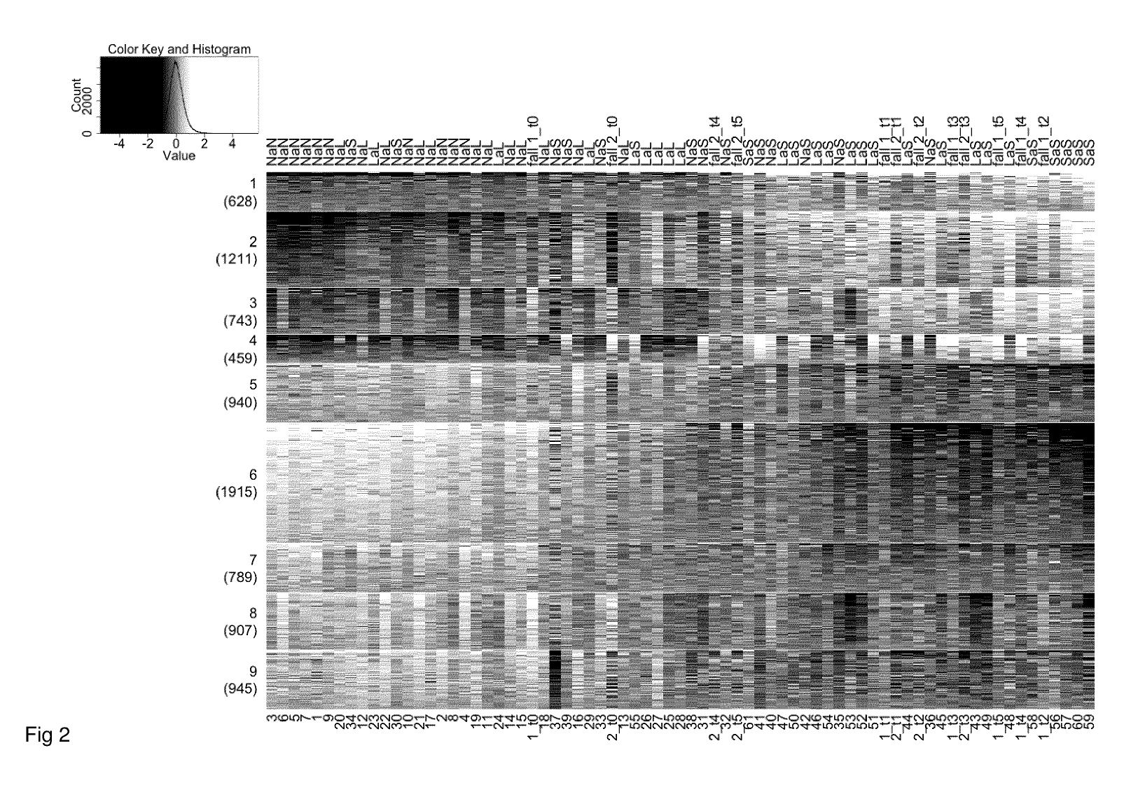

FIG. 2 shows a sorted heat map in which the expression patterns are sorted by the score (master score);

FIG. 3 shows triangles formed of various distances for the study samples;

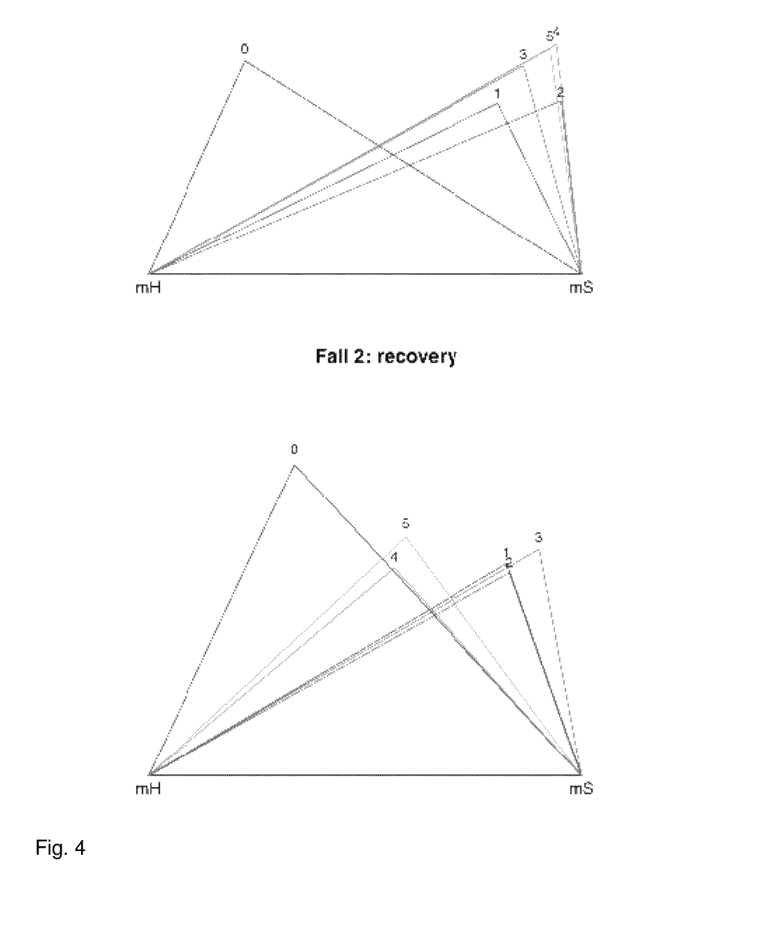

FIG. 4 shows triangles formed of various distances for two courses in patients;

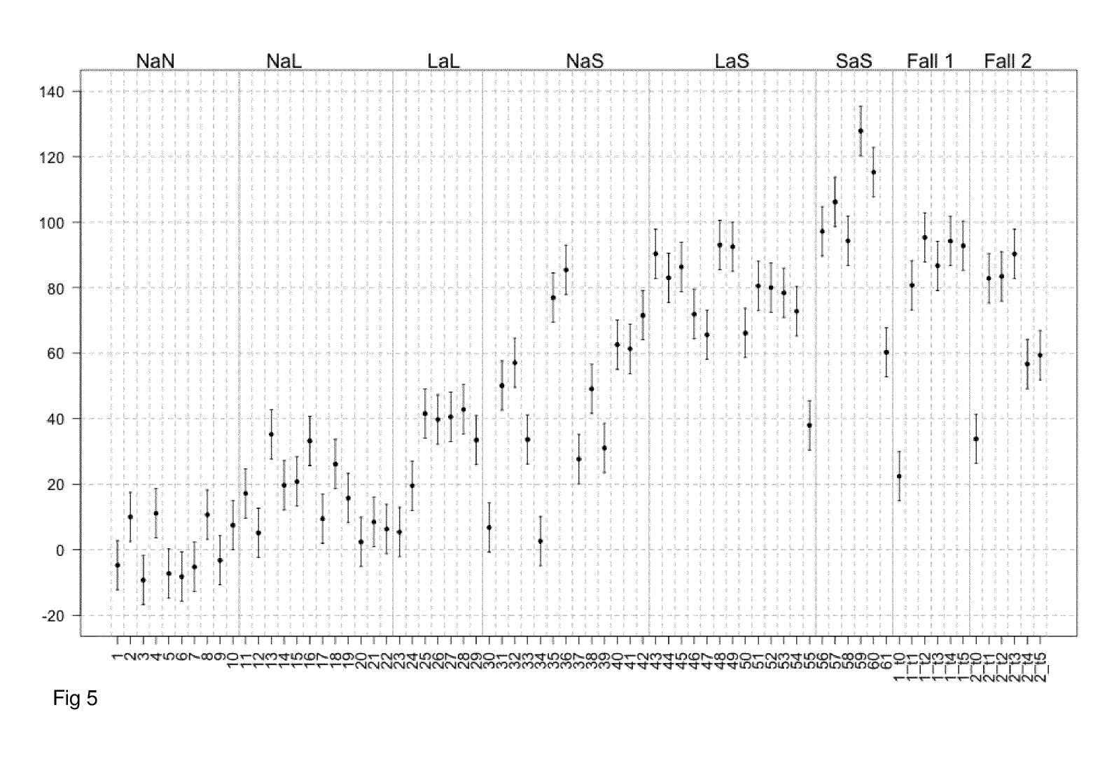

FIG. 5 shows the course of the score for all samples in the study; and

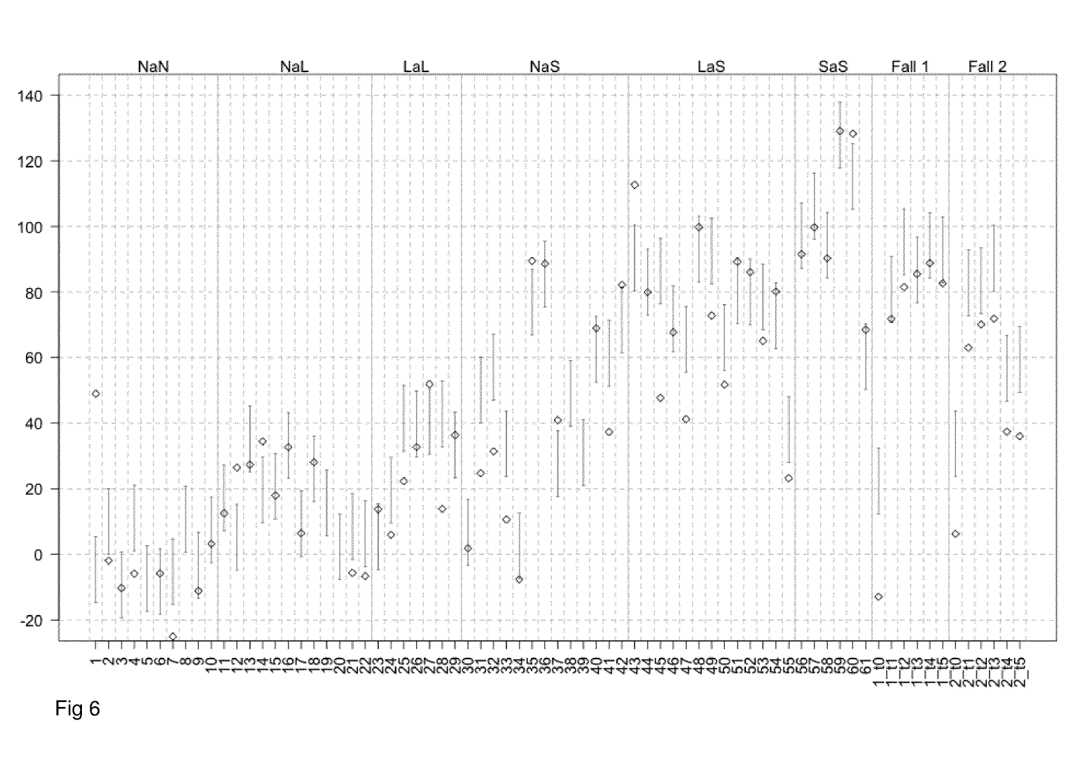

FIG. 6 shows the course of the score calculated from Delta-CT values of real time PCR gene expression measurement, as compared with the master score.

### EXAMPLES

### Example 1

### Determination of the Severity of a Host Response to a Burden of the Immune System by an Acute Inflammation

### Groups of Patients

Samples of the following test persons were included in the selection: healthy donors, patients from the hospitals for otolaryngology (ear, nose, throat—ENT) and for anesthesia and intensive care of the university hospital of Jena (KAI). The groups were allocated as follows:

SaS: 6 intensive care patients being diagnosed with severe sepsis/septic shock. The examined sample was taken on the day on which, in two independent tests (blood culture and DNA proof), the identical pathogen in the blood was confirmed.
LaS: 13 intensive care patients being diagnosed with severe sepsis/septic shock in which during the disease at least one blood sample including two independent tests (blood culture and DNA proof) was examined for pathogens, but all findings remained negative. The examined sample was taken on the first day on which a pathogen was confirmed locally.
NaS: 13 intensive care patients being diagnosed with SIRS without the indication of an infection. The examined sample was taken within the first 3 days of the SIRS diagnosis.
LaL: 7 patients of the ENT hospital with an acute peritonsillar abscess (PTA). The examined sampe was taken just prior to surgical removal of the infectious abscess. The corresponding microbiological blood analysis (as in LaS) was negative.
NaL: 8 patients of the ENT hospital with chronic tonsillitis without an acute center of infection. The examined sample was taken within the first 3 days after the tonsillectomy (surgical removal of the tonsils) was performed. The patients did not exhibit any symptoms of SIRS, at the spot where surgery was performed a sterile inflammation of the wound could be detected. Further, 4 patients with chronic non-infectious pancreatitis without SIRS were included in the group.
NaN: 7 healthy donors, 3 patients with chronic tonsillitis without an acute center of infection. The examined sample was taken prior to the tonsillectomy; the postoperative samples of these patients were not included in the NaL group.
Collect samples: In the study, samples of 2 patients each were examined over 6 consecutive days. In both cases, the first sample was taken prior to a planned surgical intervention while the subsequent samples were taken in the intensive care unit. Case 1: The patient does not recover after surgery. The inflammation-relevant parameters increase from the 2^{nd} postoperative day, on the 3^{rd} day an infection was diagnosed, the patient dies after 10 days. Case 2: The patient recovers very slowly after surgery. On the third day, some inflammation-relevant parameters increase. After medical measures have been taken the patient's condition improves, the patient is moved after a total of 6 days. The most important clinical parameters for the examined samples were collected in table 2.

TABLE 2
Summary of clinical parameters of the test persons included in the study. Features
not retrieved or parameters not determined are provided with the abbreviation n.a.

| | Sex | | | | | Survival | | | | |
|---|---|---|---|---|---|---|---|---|---|---|
| | (f: | | | | | status | | | | |
| Sample- | female | Age | Study | | | (s: released | PCT | CRP | | |
| ID | m: male) | [Years] | Group | Center | Diagnosis at admission | ns: deceased) | [ng/ml] | [mg/l] | SOFA | Pathogen |
| 1 | w | 32 | NaN | HNO | Chronic tonsillitis, | s | 0.061 | 12.67 | n.a. | None |
| | | | | | preoperative | | | | | |
| 2 | m | 22 | NaN | HNO | Chronic tonsillitis, | s | 0.089 | 1.86 | n.a. | None |
| | | | | | preoperative | | | | | |
| 3 | w | 57 | NaN | HNO | Chronic tonsillitis, | s | n.a. | 5.12 | n.a. | None |
| | | | | | preoperative | | | | | |
| 4 | m | 43 | NaN | SL | Donor | s | n.a. | n.a. | n.a. | n.a. |
| 5 | m | 24 | NaN | SL | Donor | s | n.a. | n.a. | n.a. | n.a. |
| 6 | w | 25 | NaN | SL | Donor | s | n.a. | n.a. | n.a. | n.a. |
| 7 | w | 46 | NaN | SL | Donor | s | n.a. | n.a. | n.a. | n.a. |
| 8 | m | 58 | NaN | SL | Donor | s | n.a. | n.a. | n.a. | n.a. |
| 9 | m | 38 | NaN | SL | Donor | s | n.a. | n.a. | n.a. | n.a. |
| 10 | w | 48 | NaN | SL | Donor | s | n.a. | n.a. | n.a. | n.a. |
| 11 | w | 23 | NaL | HNO | Chronic tonsillitis, | s | 0.078 | 19.74 | n.a. | None |
| | | | | | postoperative | | | | | |
| 12 | w | 19 | NaL | HNO | Chronic tonsillitis, | s | 0.058 | 15.98 | n.a. | None |
| | | | | | postoperative | | | | | |
| 13 | w | 38 | NaL | HNO | Chronic tonsillitis, | s | 0.054 | 14.72 | n.a. | None |
| | | | | | postoperative | | | | | |
| 14 | w | 58 | NaL | HNO | Chronic tonsillitis, | s | 0.035 | 94.02 | n.a. | None |
| | | | | | postoperative | | | | | |
| 15 | m | 22 | NaL | HNO | Chronic tonsillitis, | s | 0.08 | 14.53 | n.a. | None |
| | | | | | postoperative | | | | | |
| 16 | m | 22 | NaL | HNO | Chronic tonsillitis, | s | 0.066 | 60.5 | n.a. | None |
| | | | | | postoperative | | | | | |
| 17 | w | 20 | NaL | HNO | Chronic tonsillitis, | s | 0.089 | 20.05 | n.a. | None |
| | | | | | postoperative | | | | | |
| 18 | w | 46 | NaL | HNO | Chronic tonsillitis, | s | 0.059 | 46.27 | n.a. | None |
| | | | | | postoperative | | | | | |
| 19 | n.a. | n.a. | NaL | KAI | Chronic pancreatitis | n.a. | n.a. | n.a. | n.a. | n.a. |
| 20 | n.a. | n.a. | NaL | KAI | Chronic pancreatitis | n.a. | n.a. | n.a. | n.a. | n.a. |
| 21 | w | n.a. | NaL | KAI | Chronic pancreatitis | n.a. | n.a. | n.a. | n.a. | n.a. |
| 22 | m | n.a. | NaL | KAI | Chronic pancreatitis | s | 0.45 | 3.5 | 0 | n.a. |
| 23 | m | 35 | LaL | HNO | Peritonsillar abscess | s | 0.055 | 42.39 | n.a. | Streptococci in |
| | | | | | | | | | | tonsillar smear test |
| 24 | m | 40 | LaL | HNO | Peritonsillar abscess | s | n.a. | n.a. | n.a. | Streptococci in |
| | | | | | | | | | | tonsillar smear test |
| 25 | m | 28 | LaL | HNO | Peritonsillar abscess | s | 0.1 | 115.59 | n.a. | Streptococci in |
| | | | | | | | | | | tonsillar smear test |
| 26 | m | 68 | LaL | HNO | Peritonsillar abscess | s | 0.096 | 179.24 | n.a. | Streptococci in |
| | | | | | | | | | | tonsillar smear test |
| 27 | m | 30 | LaL | HNO | Peritonsillar abscess | s | 0.076 | 77.71 | n.a. | Streptococci in |
| | | | | | | | | | | tonsillar smear test |
| 28 | w | 55 | LaL | HNO | Peritonsillar abscess | s | 0.034 | 42.14 | n.a. | Streptococci in |
| | | | | | | | | | | tonsillar smear test |
| 29 | m | 70 | LaL | HNO | Peritonsillar abscess | s | 0.181 | 143.44 | n.a. | Streptococci in |
| | | | | | | | | | | tonsillar smear test |
| 30 | w | n.a. | NaS | KAI | Malign neoformation on | s | 0.14 | 2 | 5 | n.a. |
| | | | | | head of pancreas, | | | | | |
| | | | | | preoperative | | | | | |
| 31 | m | n.a. | NaS | KAI | Duodenal carcinoma, | s | 0.27 | 3.3 | 7 | n.a. |
| | | | | | preoperative | | | | | |
| 32 | m | n.a. | NaS | KAI | Carcinoma on the head of | s | n.a. | 2.1 | 2 | n.a. |
| | | | | | pancreas, | | | | | |
| | | | | | Postoperative | | | | | |
| 33 | m | n.a. | NaS | KAI | Tumor in the distal | s | n.a. | 2 | 3 | n.a. |
| | | | | | esophagus, preoperative | | | | | |
| 34 | m | n.a. | NaS | KAI | Carcinoma on the head of | s | n.a. | 5.1 | 4 | n.a. |
| | | | | | pancreas, | | | | | |
| | | | | | preoperative | | | | | |
| 35 | w | 56 | NaS | KAI | Biliary carcinoma, | s | 2.01 | 108 | 1 | n.a. |
| | | | | | Postoperative | | | | | |
| 36 | w | 70 | NaS | KAI | Partial hepatic resection, | s | 0.3 | 30.2 | 2 | n.a. |
| | | | | | postoperative | | | | | |
| 37 | w | 70 | NaS | KAI | By-pass operation after | s | 0.36 | 47.2 | 2 | n.a. |
| | | | | | cardiac infarction | | | | | |
| 38 | m | 64 | NaS | KAI | By-pass operation, | s | 0.39 | 73.7 | 4 | n.a. |
| | | | | | postoperative | | | | | |
| 39 | m | 44 | NaS | KAI | Cardiomyopathy | ns | n.a. | 156 | 4 | n.a. |
| 40 | w | 64 | NaS | KAI | By-pass operation after | s | 13.3 | 235 | 8 | n.a. |
| | | | | | cardiac infarction | | | | | |
| 41 | m | 61 | NaS | KAI | By-pass operation after | s | 1.05 | 209 | 10 | n.a. |
| | | | | | cardiac infarction | | | | | |
| 42 | m | 74 | NaS | KAI | By-pass operation after | ns | 4.43 | 154 | 12 | n.a. |
| | | | | | cardiac infarction | | | | | |
| 43 | m | 73 | LaS | KAI | Colon carcinoma | s | 1.23 | 514 | 3 | Wound: Enterobacter |
| | | | | | | | | | | cloacae, Escherichia coli |
| 44 | m | 67 | LaS | KAI | Squamous cell carcinoma, | s | 0.13 | 149 | 3 | BAL: Enterobacter |
| | | | | | right maxilla | | | | | aerogenes |
| 45 | m | 80 | LaS | KAI | Partial colon resection in | ns | 3.51 | 201 | 9 | Wound: |
| | | | | | Crohn's disease | | | | | Pseudomonas |
| | | | | | | | | | | aeruginosa, |
| | | | | | | | | | | Enterococcus faecium |
| 46 | m | 56 | LaS | KAI | Perforation of right upper | s | n.a. | 114 | 8 | Wound: Escherichia |
| | | | | | quadrant of the abdomen | | | | | coli, Enterococcus |
| | | | | | | | | | | faecalis, Klebsiella |
| | | | | | | | | | | pneumoniae |
| 47 | w | 75 | LaS | KAI | Anastomosis insufficiency | s | 4.68 | 117 | 5 | Wound: Escherichia |
| | | | | | | | | | | coli, Klebsiella |
| | | | | | | | | | | pneumoniae, Morganella |
| | | | | | | | | | | morganii, Clostridium |
| | | | | | | | | | | perfringens |
| 48 | m | 52 | LaS | KAI | decompensated hepatic | s | 64.3 | 342 | 9 | Ascites: Enterococcus |
| | | | | | cirrhosis | | | | | faecalis |
| 49 | m | 69 | LaS | KAI | Recurring secondary | s | 1.11 | 207 | 12 | BAL: Enterobacter |
| | | | | | hemorrhage and wound | | | | | cloacae, Candida krusei |
| | | | | | infection | | | | | |
| 50 | m | 78 | LaS | KAI | Arteriosclerotic cardiac | s | 1.6 | 188 | 12 | BAL: Candida |
| | | | | | disease | | | | | albicans,, |
| | | | | | | | | | | Candida glabrata, |
| | | | | | | | | | | Tracheal swab: Candida |
| | | | | | | | | | | albicans |
| 51 | m | 83 | LaS | KAI | Adenocarcinoma of the | s | 2.25 | 256 | 7 | Ascites: Enterococcus |
| | | | | | gastric antrum of an intestinal | | | | | faecalis, Candida |
| | | | | | type | | | | | glabrata |
| 52 | w | 50 | LaS | KAI | Sigma perforation in | s | 0.23 | 203 | 8 | Wound: Other gram- |
| | | | | | sigma diverticulitis | | | | | positive bacteria |
| 53 | m | 74 | LaS | KAI | Secondary hemorrhage | s | 0.42 | 179 | 11 | BAL: Staphylococcus |
| | | | | | after pertrochanteric left | | | | | aureus, Klebsiella |
| | | | | | femoral fracture | | | | | pneumoniae, Candida |
| | | | | | | | | | | albicans |
| 54 | m | 49 | LaS | KAI | Abscess of the wound on | s | 9.81 | 177 | 7 | Wound: Escherichia |
| | | | | | sigmoidostoma | | | | | coli, Enterococcus |
| | | | | | | | | | | faecium |
| 55 | m | 83 | LaS | KAI | Arteriosclerotic cardiac | s | 0.53 | 132 | 11 | BAL: |
| | | | | | disease | | | | | Stenotrophomonas |
| | | | | | | | | | | maltophilia, |
| | | | | | | | | | | Citrobacter, Klebsiella |
| | | | | | | | | | | oxytocca, Candida |
| | | | | | | | | | | albicans |
| 56 | m | 66 | SaS | KAI | Polytrauma | ns | 36.6 | 260 | 13 | Blood: Escherichia coli |
| 57 | m | 71 | SaS | KAI | Traumatic Hematothorax | ns | 2.5 | 393 | 9 | Blood: |
| | | | | | | | | | | Staphylococcus |
| | | | | | | | | | | aureus, Pan Staph. |
| 58 | m | 77 | SaS | KAI | Arteriosclerotic three- | ns | 7.2 | 245 | 13 | Blood: Enterococcus |
| | | | | | vessel cardiac disease | | | | | faecalis |
| 59 | m | 84 | SaS | KAI | Necrotizing fasciitis | s | 32.8 | 241 | 10 | Blood: Escherichia coli |
| 60 | w | 77 | SaS | KAI | Squamous cell carcinoma | s | 0.32 | 309 | 8 | Blood: |
| | | | | | of the esophagus | | | | | Staphylococcus aureus |
| 61 | w | 64 | SaS | KAI | Planned trisector ectomy | s | 1.68 | 211 | 13 | Blood: Fungi |
| | | | | | in colorectal hepatic | | | | | |
| | | | | | metastases | | | | | |
| 1_t0 | m | 65 | Case 1 | KAI | Carcinoma on the head of | ns | n.a. | 32.9 | n.a. | n.a. |
| | | | | | pancreas, | | | | | |
| | | | | | preoperative | | | | | |
| 1_t1 | m | 65 | Case 1 | KAI | Carcinoma on the head of | ns | 1.61 | 101 | 9 | n.a. |
| | | | | | pancreas, | | | | | |
| | | | | | 1st postoperative day | | | | | |
| 1_t2 | m | 65 | Case 1 | KAI | Carcinoma on the head of | ns | 1.79 | 191 | 7 | n.a. |
| | | | | | pancreas, | | | | | |
| | | | | | 2nd postoperative day | | | | | |
| 1_t3 | m | 65 | Case 1 | KAI | Carcinoma on the head of | ns | 2.21 | 273 | 9 | Wound: Morganella |
| | | | | | pancreas, | | | | | morganii, Enterococcus |
| | | | | | 3rd postoperative day | | | | | faecalis, Citrobacter |
| 1_t4 | m | 65 | Case 1 | KAI | Carcinoma on the head of | ns | 2.37 | 282 | 11 | n.a. |
| | | | | | pancreas, | | | | | |
| | | | | | 4th postoperative day | | | | | |
| 1_t5 | m | 65 | Case 1 | KAI | Carcinoma on the head of | ns | 2.02 | 331 | 10 | n.a. |
| | | | | | pancreas, | | | | | |
| | | | | | 5th postoperative day | | | | | |
| 2_t0 | m | 47 | Case 2 | KAI | Esophagus carcinoma, | s | n.a. | 16.7 | 0 | n.a. |
| | | | | | preoperative | | | | | |
| 2_t1 | m | 47 | Case 2 | KAI | Esophagus carcinoma, 1st | s | n.a. | 122 | 5 | n.a. |
| | | | | | postoperative day | | | | | |
| 2_t2 | m | 47 | Case 2 | KAI | Esophagus carcinoma, | s | 1.14 | 250 | 5 | n.a. |
| | | | | | 2nd postoperative day | | | | | |
| 2_t3 | m | 47 | Case 2 | KAI | Esophagus carcinoma, 3rd | s | 0.69 | 234 | 1 | n.a. |
| | | | | | postoperative day | | | | | |
| 2_t4 | m | 47 | Case 2 | KAI | Esophagus carcinoma, 4th | s | 0.41 | 152 | 3 | n.a. |
| | | | | | postoperative day | | | | | |
| 2_t5 | m | 47 | Case 2 | KAI | Esophagus carcinoma, 5th | s | 0.25 | 106 | 3 | n.a. |
| | | | | | postoperative day | | | | | |

### Experimental Implementation

73 RNA whole blood samples of 63 persons were measured. For this, commercial Microarray-BeadChips HumanHT-12 v3 of Illumina were used. 48803 different gene probes were located on the measuring platform used which gene probes represent the entire human genome independent of the tissue.

The samples were processed and measured using the following steps:

1. Isolation and stabilization of the total RNA from whole blood samples: the base material for analyzing the transcriptome of blood samples is 2.5 ml of whole blood. The whole blood was taken in a PAXgene tube (PAXgene Blood RNA Tube PrAnalytiX #762165 (Becton Dickinson)) and stored until reprocessing at −80° C.
2. Standard automatic RNA isolation from PAXgene blood samples: The QIAcube (Qiagen, Hilden) as well as the PAXgene Blood RNA Kit (PreAnalytiX #762174) were utilized, thereby using the program “PAXgene Blood RNA (CE)” for isolating the total RNA. After the end of the listing the elution tubes including the RNA isolates were sealed. Any remaining DNase enzyme activities were inactivated by heating the samples for 5 minutes at 65° C., the samples were then immediately cooled on ice and stored at −80° C.
3. Quality control of the total RNA: Examination of the isolated RNA is an important measure for ensuring the quality of the hybridization results. Merely an intact RNA is able to provide excellent hybridization results. Examination of the integrity of the isolated total RNA was done in a capillary-electrophoretic manner using the Bioanalyzer 2100 of Agilent Technologies thereby making use of the RNA 6000 Nano LabChip Kit (Agilent Technologies, catalog number 5067-1511) in accordance with the specifications of the manufacturer. A RIN value around 7.5 on a scale of 1-10 is regarded as guidance level. All samples used reached a RIN>5, which is sufficient for the purpose of gene expression analysis (cf. Fleige and Paffl, 2006).
4. Reduction of the globin mRNA: For improving the sensitivity of gene expression measurements in whole blood the highly abundant globin mRNA was recommended. To this end, the “GLOBINclear TM-Human” kit of Ambion/Applied Biosystems # AM 1980) was used. According to estimations of the manufacturer the globin transcripts with a proportion of 70% of all mRNAs in blood considerably overlay less present transcripts. 1 μg of total RNA of each sample was used for processing.
5. Amplification of total RNA to cRNA: Preparation of the globin-reduced RNA for hybridization was done using the “Illumina TotalPrep RNA Amplification kit” (AMIL 1791) of Ambion/Applied Biosystems in accordance with the specifications of the manufacturer, thereby using an amount of 500 ng. The cRNA eluates were cooled on ice, the cRNA concentration was measured spectrophotometrically on the Nanodrop ND-2000.
6. Hybridization on Illumina BeadChips: Pangenomic Illumina BeadChips, Version human HT-12v3 were used. 750 ng of the cRNA samples in a volume of 5 μl per array were applied and hybridized over night at 58° C. Signal detection of successfully hybridized probes is done via CY3-streptavidin staining (GE-Amersham) in accordance with the specifications of the manufacturer. Illumina listing: Whole-Genome Gene Expression with IntelliHyb™ Seal; Experienced User Card; Part #11226030 Rev. B, Illumina Inc. Read-out of the fluorescence signals was effected by using the Illumina® Bead Array Reader 500 and the corresponding Illumina “BeadScan” software (version 3.6.17).
7. Image analysis of the micro arrays: The BeadChips stained with fluorescent color are scanned with the Illumina® Bead Array Reader. The resulting images are analyzed by way of the Illumina® “Genome-Studio” software (Genome Studio 2009.2 version). For a first assessment of the hybridization technical control signals are retrieved. A first qualitative review is gained by way of the number of detected genes and the average signal strength per array. The raw data are subjected to quality control and statistical analysis.

### Data Analysis and Statistical Evaluation

Data analysis was performed using the freely available software R Project Version R 2.8.0 (R.app GUI 1.26 (5256), S. Urbanek & S. M. lacus, R Foundation for Statistical Computing, 2008), which is available under www.r-project.org [R Development Core Team (2006)]. The software is preferably used in analyzing gene expression data since it provides for numerous algorithms for processing such kind of data. In particular, the following software packages could be used in our study: lumi for reading in Illumina measuring values and the related gene annotation, vsn for data normalization (both in Du et al., 2008), fdrtool for determining the false-positive rate (Strimmer, 2008), and stats for performing statistical significance tests for clustering and visualizing the results.

The analysis was performed using the following steps. The raw data as well as additionally available annotation data were read in. On account of variations for technical reasons in processing the samples and deviations in the quantity of reagents, the fluorescence intensities of different samples as a rule cannot be compared to one another directly. To make this possible, such variations are compensated for by suitable standardization, a variance-stabilizing transformation being used [Huber et al., 2002].

Normalized data and measuring data of which their logarithms were taken with regard to basis 2, were included in the data analysis. In the statistical analysis 61 samples of the 6 study groups were examined. The gene expression between these groups was compared by way of one-way analysis of variance [Mardia et al., 1979]. In so doing, it was examined whether there is a significant difference between at least 2 of the analyzed groups. The corresponding significance test was computed, gene by gene, for 18517 gene probes that provided a signal of detectable intensity for at least one RNA sample. The number of false-positive tests was determined by way of a false discovery rate (FDR) [Storey et al. (2003)]. For each gene probe the so-called q value was calculated, which is defined as the minimum FDR under which the probe appears as being significantly modified. The method used for FDR control estimated 87.5% of the gene probes as being significantly modified. This corresponds to 16204 probes of a pool of 18517 examined probes.

Further, the gene expression patterns of the 6 groups of patients were compared by pairs by way of the t test. The test was applied gene by gene for a total of 15 group combinations. In so doing, the same selection of gene probes was considered and an FDR control performed that was described for the one-way analysis of variance. The results were summarized in table 3.

TABLE 3
Summary of false-positive rates (FDR) in comparing all study groups by pairs.
The table indicates how many gene probes, by comparison, have reached an FDR value
smaller than the indicated threshold. This interpretation is explained using an
example: In comparing NaS vs. NaL a group of 265 genes with an FDR of 1% and less
was determined, i.e. of the 265 gene probes 2-3 are false-positive.

| | | | | | | | Proportion of |
|---|---|---|---|---|---|---|---|
| | <0.01% | <0.1% | <1% | <2.5% | <5% | <10% | modified gene |
| FDR | (n) | (n) | (n) | (n) | (n) | (n) | probes [%] |
| LaS vs. NaN | 3412 | 5443 | 8309 | 9901 | 11314 | 13315 | 70.0 |
| SaS vs. NaN | 375 | 1909 | 6285 | 8518 | 10301 | 12549 | 69.4 |
| LaS vs. NaL | 2098 | 3962 | 6696 | 8232 | 9746 | 11850 | 65.5 |
| SaS vs. NaL | 137 | 912 | 4317 | 6531 | 8385 | 10591 | 62.7 |
| NaS vs. NaN | 0 | 73 | 3650 | 5654 | 7585 | 9932 | 59.8 |
| NaS vs. NaL | 0 | 0 | 265 | 2591 | 4799 | 7672 | 56.1 |
| LaS vs. LaL | 1 | 19 | 1019 | 2499 | 4416 | 6864 | 53.7 |
| SaS vs. LaL | 0 | 0 | 688 | 2620 | 4789 | 7276 | 53.3 |
| LaL vs. NaN | 0 | 0 | 527 | 2270 | 4398 | 7080 | 52.5 |
| SaS vs. NaS | 0 | 0 | 119 | 684 | 2048 | 4599 | 50.9 |
| LaL vs. NaL | 0 | 0 | 1 | 1 | 526 | 2589 | 44.5 |
| LaS vs. NaS | 0 | 0 | 0 | 0 | 0 | 522 | 33.1 |
| SaS vs. LaS | 0 | 0 | 0 | 0 | 0 | 0 | 27.8 |
| NaS vs. LaL | 0 | 0 | 0 | 1 | 6 | 154 | 27.0 |
| NaL vs. NaN | 0 | 0 | 5 | 86 | 236 | 841 | 26.0 |

As follows from the table (last column), the clearest differences are to be found between patients suffering from sepsis (LaS and SaS) and the groups NaN (no inflammation) and NaL (non-infectious local inflammation). In contrast thereto, there were little distinct differences between the group pairs NaS and LaL, SaS and LaS, as well as LaS and NaS. In comparing the amount of gene probes that, by comparison of LaS vs. NaS and NaS vs. LaL reached an FDR<0.1, no gene probes could be found that in an infection showed the same modification in gene expression. Thus, no gene probes could be detected that show an infection-specific expression pattern. It should be mentioned that also further statistical comparisons, inter alia the two-way analysis of variance [Mardia et al., 1979], between the groups NaL, NaS, LaL and LaS, as well as a comparison of groups LaL and LaS vs. NaS, lead to the same negative result.

Surprisingly, the comparisons by pairs make the following arrangement of the study groups possible: (1) NaN, (2) NaL, (3) LaL, (4) NaS, (5) LaS, (6) SaS. The arrangement is distinguished by the fact that the adjacent groups statistically differ only little in their expression. More exactly, in the statistical comparison, a sufficient number of modified gene probes were found, gene probe groups with a sufficiently small false-positive rate (approx. 5%) could not be found between the adjacent groups. The phenomenon occurs if average group values differ from each other, but the dispersion of the groups is high, so that this leads to overlapping of the two groups compared. The farther the groups are apart, the greater are the differences. It should be mentioned that a directed arrangement of the study groups was possible without a determination of gene expression.

In order to depict the uncovered change of gene expression within the study groups 8537 gene probes from the first statistical comparison in which group differences were generally examined by way of a one-way analysis of variance, were included in the further analysis. The selection was based on an estimation of a false-positive rate of 0.3%. This is interpreted statistically such that approximately 26 probes of a selection list of 8537 probes (i.e. 0.3%) are false-positive. Any extension of the selection would lead to a higher false-positive rate. The selected 8537 gene probes address a total of 7694 different RNA transcripts.

The gene expression of these gene probes within the study was grouped, based on their similarity, in 9 gene clusters by way of a k-means algorithm (Hartigan and Wong, 1979). For this, the R function kmeans was used, which standardizes the expression signals before hand gene by gene with regard to an average value and dispersion. Estimation of the number of clusters was done in accordance with the 2^{nd} derivation of the error function (cost function), resulting from a repetition of the clustering method for 1 to 20 clusters [Goutte et al., 1999].

The groups of patients were arranged in the sequence (1) NaN, (2) NaL, (3) LaL, (4) NaS, (5) LaS, (6) SaS. At the end, the 12 gene expression patterns from the 8537 gene probes of the collect samples of the two patients were added. The sorted expression matrix was visualized in a so-called heatmap. In FIG. 1, each line represents a gene probe and each column an RNA sample. The relative alteration of gene expression is depicted in gray shades. Thus, dark gray to black encodes a lower expression and light gray to white a higher expression than determined as an average per gene probe. The clusters were numbered from 1 to 9, the number in brackets below the cluster number indicating the number of gene probes in a cluster. Within a cluster, the gene probes were sorted in a declining manner, so that probes with larger differences within the study groups are located at the upper end of the cluster.

From FIG. 1 it is apparent that there is a trend in the expression from left to right. From group NaN to group SaS the expression in gene clusters 1 to 4 augments and declines in gene clusters 5 to 9. However, also high variability within the individual groups, particularly the NaS group, is shown. There are flowing transitions between individual groups, the groups overlap each other.

The average difference between groups NaN (no acute inflammation) and SaS (patients suffering from SIRS with a confirmed infection in the blood stream) is the most distinct one. This difference was quantified in the next step by way of a clearance (cf. the next section). All other samples were arranged according to their distance to these two groups. In the sorted heat map which is depicted in FIG. 2, such arrangement is visualized. Samples that resembled more the pattern of healthy persons, were sorted to the left and samples that resembled more the pattern of intensive care patients with an infection in the blood were sorted to the right.

The sorted heat map shows that the samples of the patients for the most part were sorted according to their group affiliation in the sequence as listed above, or were mixed into adjacent groups. However, individual samples are sorted into clearly higher or lower ranks than most group representatives. The gene expression pattern of the heat map shows that the gene activity of NaN to SaS increases and decreases at the same time. The individual gene clusters merely distinguish in the progression of the deviation.

The result points out to the fact that the gene expression particularly reflects the strength of the host response to the burden of an organism caused by an inflammation. In fact, an infection in the blood stream with systemic inflammation signifies the greatest stress to the immune system. Similar stress will arise through a local infection if the immune system is not capable of battling pathogens in the source of infection. But also through a traumatic event, e.g. a severe operation, the immune system, for a short time, may be used to the full up to the limit of the ability to withstand stress. Moreover, the gene expression shows the severity of the host response in case of lesser stress caused by local inflammation.

As was mentioned already, the selected genes are merely divided into 2 groups. In the first group of 3041 gene probes (36%), the gene expression increases with the burden of the immune system (cluster 1 to 4) while the gene expression decreases in the other group of 5496 gene probes (64%) (clusters 5 to 9). On account of known references [(cf. Calvano et al. (2005) and Foteinou et al., 2008], one would speak of a (pro- and anti-) inflammatory response in case of an increase in the gene expression, and of an energetic response in case of a decrease in the gene expression. The energetic exhaustion in case of ongoing inflammation will lead to an ungovernable immune response.

### Quantifying the Severity of the Host Response

Sorting of the heat map was done in accordance with the following score. Let X be the corresponding gene expression vector for an RNA sample, which sums up the expression signals of the selected gene probes. The Euclidean distance between the two vectors X₁ and X₂ be referred to as d(X₁, X₂). Furthermore, let cor(X₁, X₂) denote the correlation coefficient between X₁ and X₂ according to Pearson, which corresponds to the cosine value of the angle between X₁ and X₂ [Mardia et al., 1979].

The score is calculated in accordance with the following formula:

$$score ( X ) [ \% ] = \frac{100 \%}{d ( mS , mH )} \cdot cor ( X - mH , mS - mH ) d ( X , mS - mH ) \qquad Formula 1$$

mH and mS being the two gene expression vectors that sum up the average values of groups NaN (mH) and SaS (mS) gene by gene.

The score may be illustrated as follows. From the distances d(mS, mH), d(mS,X) and d(X,mH) a triangle is formed the corners of which, for reasons of simplification, are denoted by X, mH and mS (cf. FIG. 3). The value

L_{X}=cor(X−mH,mS−mH)d(X,mS−mH)

defines the position of the foot of the perpendicular from corner X to the straight line which is defined by d(mS, mH). The value of L_{X} corresponds to the distance of mH and the foot of the perpendicular. It also indicates how far corner X of the triangle is away from corner mH, the height of the triangle is not assessed.

Finally, the score, which is defined by formula 1, indicates the relative proportion of distance L_{X} with regard to distance d(mS, mH). In fact, one obtains from formula 1, for X=mH: score (mH)=0% and for X=mS: score(mS)=100%.

In FIG. 3, sample 3 is farther apart from mS than from mH and receives a score value of −9.3%. Sample 59 is farther apart from mH than from mS and receives a score value of 127.8%. Finally, sample 37 is between mH and mS and receives a score value of 27.7%. The value of 50% would be allocated to a sample that has an equal distance to mH and mS.

The distance d(mS, mH) is composed in an additive manner of the distances of the individual gene probes. If the total distance is divided into two components, the one component d⁺(mS, mH) being calculated from the gene probes of clusters 1 to 4, and the other component d⁻(mS, mH) being calculated from the gene probes of clusters 5 to 9, information is gained on the share of the expression increase and decrease of the total deviation. In the data set examined by us the increase the increase that is represented by 36% of the gene probes, was 51% of the total distance. The decrease, represented by 64% of all probes, was 49% of the total distance. Thus, on average, the expression of one gene probe from clusters 1 to 4 was declining more than in clusters 5 to 9. The total ratio of increase and decrease was approximately equal. If the ratio of increase and decrease is calculated for each sample, information is gained on the progression of the deviation in the direction shown.

FIG. 4 shows how the triangle formed by the above-indicated distances for the two cases of patients is moved in the course of the disease, the index above the tip of the triangle indicating the day of taking the sample, and 0 indicating the pre-operative sample.

FIG. 5 depicts the score for all study samples. The study groups and the two courses were arranged as in FIG. 1. The black points mark the score value, the bars an upward or downward deviation of 7.5% percentage points.

It should be noted that the proposed score is not the only one with which difference in the gene expression of the study groups may be quantified. The advantage of the score consists in that a relative measurement is defined thereby, i.e. a percentile proportion of the difference determined between the gene expression of the group without acute inflammation and SIRS patients with a blood infection. The score is independent of the measuring platform and the number of gene markers used.

Although merely simultaneous increase and decrease of gene activity is quantified by the score, it is calculated from the expression of several thousands of genes. The cause for this lies in the phenomenon examined. The used genes in general are responsible for different processes. Therefore, for an individual gene, the deviation in the expression from a healthy person may have various causes. However, the flocking behavior of many genes in the declared direction reflects the quantitative extent of an immune burden.

From the examples in which the post-operative condition was observed for 2 patients, it is to be seen that the score captures the current extent of a host response. Therefore, it may be used for observation/monitoring. In fact, the value of the score for a patient within 6 days changes from approximately 20% to approximately 90% of percentage points. Moreover, it is suited for generally assessing an immune burden. Indeed, the pre-operative samples of the two patients exhibit an increased score value of more than 20%. This may be one of the causes for a post-operative course abundant with complications.

### Example 2

### Determination of the Severity of the Host Response in Patients with an Acute Inflammation by Way of a Reduced Number of Markers

### Selection of Gene Markers Based on Simulations

For determining the intensity of a host response a high number of gene markers were used. In general, the examined genes are responsible for different processes and for the individual gene; the deviation in the expression from a healthy person has various causes. The more genes are observed, the better a different cause than a burden of the immune system may be excluded for a deviation in the expression. The observation of all relevant genes totally excludes other causes. However, there is the well-founded assumption that even a reduced number of gene probes would reflect the stress condition of an immune system sufficiently enough. However, a reduction in the number of genes is only plausible if the resulting score lies sufficiently close to the original score (master score). In our study, the master score could be determined with high accuracy from the 4372 gene probes the average expression of which between the two study groups amounted to at least 0.8. In fact, the deviation amounted to no more than 1 percentage point. If the score was calculated from the remaining 4165 gene probes the deviation was below 8 percentage points.

Since there is no algorithm for selecting a marker, it is obvious to examine via computer simulations as to whether there is a preferred number and amount of genes that depict the master score. In order to estimate the number of required gene probes, a maximum of 1500 out of the 8357 gene probes of the master score was randomly selected in first simulations. 36% thereof were from clusters 1 to 4, and 64% from clusters 5 to 9. For each selection, the score was calculated for all 73 RNA samples in accordance with formula 1. Those sets of gene probes were retained the score of which did not deviate from the master score for more than ±7.5 percentage points. In 500 repetitions, 241 of such sets of gene markers were found. Those sets represented all 8375 gene probes. The shortest set included 138 gene probes. Within the next 5000 repetitions, a maximum of 150 gene probes per run was randomly selected their distribution among the clusters was as that in the first run. As a result, we obtained 24 sets with a length of 86 to 148 gene probes; the corresponding score values did not deviate from the master score for more than ±7.5 percentage points. The results of the pre-examination demonstrate that the number of gene markers may be reduced considerably in case a reasonable deviation from the master score is accepted. In the simulation described in the following the maximum number of gene probes was reduced further. The results of these simulations were collected in Table 5.

The approach in the first simulation was as follows. The expression matrix of 8357 gene markers and 61 expression vectors from the 6 study groups was subjected to an analysis of main components (Mardia et al., 1979). For this, the R function prcomp was used. 512 gene probes were selected that correlated the strongest with the main component. This included 183 (36%) from gene clusters 1 to 4 and 329 (64%) from gene clusters 5 to 9. Thus, the selection amount was reduced to gene probes that represent the clearest the examined trend in the change in gene expression, wherein the increase and decrease were of the same ratio as in the primary selection.

From this pre-selection (512 gene probes) 40 to 50 gene probes were randomly selected in 5000 simulation steps and from those gene probes the score was calculated for all 73 gene patterns in accordance with formula 1. The selection was discarded if the absolute difference between the master score and the new score for at least one sample was more than 7.5 percentage points. Otherwise, the selection was stored. The simulation provided 2 sets of gene probes that fulfilled the condition. Those sets were described as set 1 and set 2 in Table 4 via the corresponding sequence number. They included 49 and 47 gene sequences.

In the next 5000 simulation steps those gene probe tuples were retained in which for 70 samples (95%) the reduced score did not deviate from the master score for more than 7.5 percentage points. In this simulation step 14 different combinations were found. The amounts, described as set 3 to set 16 in Table 4 via the corresponding sequence number, included 46 to 49 gene sequences.

In a third simulation, the number of randomly selected probes was reduced to a maximum of 20 and the selection procedure was repeated 50000 times. Likewise, those gene probe tuples were retained in which for 70 samples (95%) the reduced score did not deviate from the master score for more than 7.5 percentage points. In this simulation, 20 different combinations were found that fulfilled the selection condition.

The amounts described as set 17 to set 36 in Table 4 via the corresponding sequence number, included 18 to 20 gene sequences. The results of the simulation show that the score may be determined sufficiently accurate even from a clearly reduced number of probes. In fact, an error bar of 15 percentage points (which corresponds to an error of ±7.5 percentage points) appears acceptable if it leads to reducing the number of gene markers considerably. It is to be taken into consideration that also the original score is subject to random deviations and depends on the underlying random sample. Moreover, the simulations show that there are no preferred gene probes that determine the score. Indeed, different gene probe tuples lead to a similar estimation of the master score. In fact, the estimation includes a total of 423 different sequence numbers.

TABLE 4
Summary of the gene probe tuples fulfilling the described selection criteria in simulations. The sets
were numbered from 1 to 36, the number n in brackets indicating the number of sequences in the set. The
subsequent numerical order indicates the corresponding sequence numbers from the sequence listing.

Set 1 (n = 49) 508, 553, 611, 679, 734, 769, 851, 860, 871, 896, 1117, 1263, 1646, 1647, 1648, 1675, 1688,
1975, 2011, 2077, 2415, 2516, 2560, 2581, 3381, 3491, 3820, 3947, 4156, 4230, 4506, 4576, 5012, 5235,
5614, 5730, 5803, 5873, 6114, 6262, 6265, 6301, 6689, 6738, 6820, 6847, 6879, 7069, 7230
Set 2 (n = 47) 160, 309, 374, 428, 462, 911, 937, 1039, 1092, 1105, 1458, 1533, 1604, 1895, 1917, 1997,
2002, 2055, 2242, 2332, 2369, 2386, 2427, 2516, 2541, 2560, 2785, 3359, 3407, 3624, 4230, 4587, 4636,
5164, 5235, 5247, 5371, 5776, 6278, 6328, 6497, 6636, 7156, 7201, 7230, 7314, 7450
Set 3 (n = 48) 10, 366, 411, 462, 493, 495, 567, 1204, 1226, 1409, 1414, 1449, 1487, 1583, 1724, 1744,
2013, 2055, 2064, 2208, 2248, 2692, 2891, 3051, 3624, 4156, 4205, 4510, 4587, 4923, 5176, 5373, 5400,
5435, 5873, 5912, 5954, 6041, 6073, 6247, 6301, 6478, 6525, 6923, 7207, 7450, 7670, 7681
Set 4 (n = 47) 160, 359, 441, 493, 522, 541, 652, 691, 1128, 1408, 1583, 1651, 1652, 1664, 1688, 2002,
2077, 2248, 2273, 2415, 2676, 2690, 2755, 2876, 3053, 3623, 4216, 4327, 4525, 4587, 4765, 4870, 5013,
5164, 5431, 5614, 5950, 6098, 6265, 6432, 6497, 6981, 7062, 7202, 7314, 7450, 7607
Set 5 (n = 49) 97, 428, 441, 543, 611, 851, 1136, 1384, 1533, 1868, 1997, 2077, 2183, 2208, 2226, 2260,
2329, 2386, 2475, 2686, 2690, 2876, 3054, 3821, 4000, 4357, 4479, 4530, 4636, 4765, 4923, 5013, 5137,
5204, 5760, 5776, 5819, 5873, 5908, 6005, 6099, 6242, 6417, 6499, 6585, 6847, 7450, 7670, 7681
Set 6 (n = 48) 10, 97, 359, 475, 495, 627, 928, 1039, 1117, 1248, 1384, 1408, 1472, 1652, 1675, 1744, 1868,
1918, 2370, 2423, 2537, 2742, 2865, 3051, 3086, 3408, 3916, 4030, 4078, 4274, 4294, 4362, 4751, 5129,
5235, 5247, 5431, 5734, 5803, 5811, 5908, 5950, 6005, 6417, 6497, 6525, 6923, 7456
Set 7 (n = 49) 32, 160, 383, 414, 493, 611, 652, 679, 734, 885, 896, 946, 1177, 1640, 1650, 1704, 1882,
2077, 2248, 2250, 2260, 2415, 2561, 3086, 3488, 3623, 3624, 4135, 4156, 4160, 4510, 4525, 4530, 4742,
5137, 5204, 5247, 5730, 5950, 6114, 6210, 6225, 6430, 6478, 6497, 6545, 6668, 7314, 7607
Set 8 (n = 48) 359, 383, 515, 538, 544, 691, 769, 813, 1024, 1039, 1092, 1409, 1519, 1640, 1649, 1665,
1696, 1731, 1744, 2167, 2183, 2226, 2260, 2273, 2425, 2516, 2618, 2634, 2672, 3051, 3168, 3202, 4160,
4754, 4966, 5373, 5465, 5493, 5541, 5574, 5912, 6005, 6216, 6432, 6636, 6748, 6847, 7423
Set 9 (n = 46) 160, 352, 544, 691, 802, 885, 1126, 1147, 1163, 1336, 1416, 1639, 1969, 2002, 2058, 2077,
2183, 2331, 2332, 2426, 2526, 2742, 2855, 2860, 2891, 3054, 3138, 3488, 3947, 4560, 4576, 4707, 4776,
5235, 5371, 5400, 5431, 5760, 5873, 6247, 6301, 6417, 6673, 6820, 7447, 7604
Set 10 (n = 49)8, 164, 462, 494, 495, 510, 545, 567, 611, 679, 941, 1039, 1105, 1128, 1147, 1318, 1533,
1649, 1918, 1973, 1975, 2011, 2077, 2080, 2370, 2537, 3051, 3202, 3676, 4274, 4587, 4928, 5204, 5373,
5431, 5465, 5541, 5734, 5908, 5912, 5950, 6278, 6417, 6497, 6668, 6673, 7156, 7230, 7670
Set 11 (n = 46)89, 97, 160, 355, 359, 366, 374, 411, 462, 475, 515, 538, 543, 691, 1384, 1647, 1649, 1651,
1724, 2011, 2058, 2064, 2242, 2369, 2859, 3414, 4000, 4742, 4765, 4870, 4966, 5040, 5232, 5247, 5276,
5373, 5431, 5760, 5873, 5954, 6417, 6419, 6497, 6545, 6636, 7484
Set 12 (n = 49)8, 89, 515, 543, 585, 769, 969, 1126, 1163, 1526, 1583, 1639, 1744, 2019, 2393, 2415, 2453,
2618, 2690, 2692, 2810, 2855, 2863, 3153, 3158, 3190, 3408, 4000, 4083, 4104, 4248, 4479, 4491, 4550,
4661, 4877, 4995, 5176, 5276, 5599, 5695, 6073, 6114, 6265, 6417, 6499, 6585, 6632, 6673
Set 13 (n = 48)414, 538, 946, 1263, 1384, 1512, 1895, 2077, 2248, 2260, 2516, 2676, 2975, 3168, 3414,
4083, 4274, 4776, 4800, 4919, 4923, 5179, 5204, 5431, 5493, 5541, 5619, 5695, 5819, 6005, 6073, 6099,
6210, 6247, 6265, 6350, 6417, 6432, 6499, 6536, 6545, 6636, 6668, 6689, 7040, 7062, 7472, 7604
Set 14 (n = 47)383, 428, 538, 553, 691, 814, 871, 896, 911, 937, 1426, 1639, 1685, 1688, 1983, 2093, 2253,
2260, 2454, 2516, 2587, 2672, 2761, 2865, 2975, 3086, 3781, 4000, 4030, 4308, 4510, 4636, 4923, 5137,
5235, 5574, 5776, 5819, 5908, 6226, 6278, 6417, 6632, 7202, 7230, 7315, 7456
Set 15 (n = 46)97, 366, 383, 802, 1426, 1514, 1558, 1685, 1744, 1975, 2011, 2013, 2369, 2415, 2454, 2510,
2516, 2577, 2587, 2759, 2968, 3168, 3364, 3641, 3780, 4083, 4230, 4294, 4587, 4638, 4817, 5040, 5164,
5276, 5371, 5465, 5541, 6073, 6098, 6114, 6184, 6216, 6497, 6515, 7062, 7202
Set 16 (n = 49)10, 504, 541, 553, 567, 652, 802, 1024, 1092, 1136, 1197, 1519, 1646, 1647, 1648, 1652,
2055, 2058, 2260, 2273, 2330, 2331, 2415, 2491, 2581, 2618, 2676, 2742, 3053, 3408, 3652, 3915, 4216,
4870, 5235, 5641, 5695, 5954, 6114, 6278, 6419, 6461, 6791, 6820, 6847, 6923, 7428, 7604, 7670
Set 17 (n = 20)10, 160, 428, 871, 941, 1136, 1197, 1416, 1558, 1786, 1951, 2386, 2510, 2560, 3488, 3652,
3781, 5176, 5400, 6515
Set 18 (n = 20)871, 1163, 1414, 1416, 1426, 1487, 2001, 2055, 2369, 2386, 2552, 2577, 2865, 3051, 4550,
4577, 5614, 6098, 7369, 7423
Set 19 (n = 20)567, 958, 2226, 2250, 2260, 2427, 2516, 3364, 4030, 4135, 5235, 5574, 5950, 6114, 6226,
6267, 6278, 6418, 7069, 7518
Set 20 (n = 20)409, 1647, 1648, 1770, 1883, 1951, 2013, 2386, 2423, 3152, 3491, 4205, 4577, 4661, 4765,
4919, 7428, 7604
Set 21 (n = 20)355, 480, 494, 667, 1492, 2475, 2855, 2948, 3155, 3158, 3408, 3780, 4661, 5113, 5232, 5368,
5574, 6114, 6419, 6499
Set 22 (n = 19)769, 1163, 1472, 2077, 2370, 2759, 3488, 3567, 3737, 3780, 4230, 4245, 4274, 4550, 5950,
6497, 7069, 7109, 7681
Set 23 (n = 20)160, 164, 355, 411, 1106, 1408, 1675, 1679, 2386, 2453, 2516, 2810, 3168, 3202, 3652, 4230,
5574, 5986, 7428, 7484
Set 24 (n = 19)10, 164, 885, 1263, 1318, 1416, 1492, 1508, 1647, 1951, 2250, 2560, 2785, 2827, 3086, 4506,
5137, 5575, 5954
Set 25 (n = 19)32, 522, 679, 1519, 2001, 2491, 2516, 2676, 3412, 3737, 4205, 4294, 4560, 5235, 5954, 6005,
6114, 6499, 6525
Set 26 (n = 19)958, 1449, 1472, 1582, 2332, 2516, 2552, 2891, 2975, 3168, 3190, 3683, 3820, 3947, 4245,
4530, 7040, 7069, 7145
Set 27 (n = 20)428, 515, 544, 562, 567, 1263, 2002, 2332, 2526, 3438, 4577, 4754, 5574, 5614, 5912, 6328,
6515, 7156, 7423, 7456
Set 28 (n = 20)355, 508, 937, 1263, 1973, 2002, 2510, 4078, 4156, 4550, 4673, 4817, 5247, 5368, 5730,
6005, 6247, 6515, 7201, 7207
Set 29 (n = 20)10, 544, 871, 1408, 1487, 1649, 2002, 2415, 2690, 2859, 2975, 3126, 4577, 4636, 5541, 6073,
6417, 6432, 6866, 6879
Set 30 (n = 20)896, 1248, 1318, 1472, 1786, 1830, 1983, 2386, 2865, 2975, 3641, 3916, 4030, 4530, 4995,
5472, 5619, 6099, 6247, 6265
Set 31 (n = 20)355, 462, 1416, 1983, 2011, 2183, 2248, 2618, 3190, 3412, 4490, 4576, 4776, 4923, 5164,
6101, 6114, 6278, 7314, 7369
Set 32 (n = 20)310, 1226, 1895, 2248, 2427, 2516, 2552, 2690, 3086, 3438, 3915, 4216, 4587, 5235, 5276,
5954, 6265, 6478, 6515, 7207
Set 33 (n = 20)493, 584, 633, 937, 2330, 2377, 2491, 2587, 3153, 3683, 4216, 4248, 4530, 6114, 6419, 6478,
6525, 6689, 7202, 7456
Set 34 (n = 20)10, 359, 383, 478, 626, 1472, 1487, 1647, 2475, 3683, 3780, 4490, 4636, 5179, 5247, 5371,
5950, 6748, 6923, 7670
Set 35 (n = 20)97, 626, 1039, 1163, 1426, 1617, 1704, 2002, 2248, 2690, 3168, 4216, 4638, 5247, 5614,
5950, 6265, 6461, 6632, 7428
Set 36 (n = 20) 366, 414, 544, 734, 1263, 1416, 2167, 2208, 2250, 2370, 2491, 2526, 2855, 3190, 3488, 4083,
4248, 6673, 6845, 6847

It should be noted that individual sets 1 to 36 indicated in table 4 as well as the two following sets: Set 37 (n=10): SEQ-ID numbers: 1983, 507, 5431, 3043, 1665, 5776, 2902, 6585, 3167 and 745; and Set 38 (n=7): SEQ-ID numbers: 1983, 507, 5431, 3043, 1665, 5776 and 7695 each taken by itself constitute preferred embodiments of the present invention with which a score value may be obtained that merely lies within the limits of the deviation from a master score as indicated in the present invention, so that the individual sets 1 to 38 each allow for exact statements in the sample of a test person or patient with regard to the in vitro determination of the severity of the host response of a patient who is in an acutely infectious and/or acutely inflammatory condition.

### Preferred Gene Marker Tuples

In former non-pre-published patent application DE 10 2009 044 085 of applicant gene expression markers were found that are indicative of an infection in patients with a systemic inflammatory response syndrome (SIRS) (DE 10 2009 044 085). Of the 13 gene markers examined therein, 6 to 7 markers are found again in the list of 8537 gene probes examined herein. An explanation for this is that SIRS patients with an infection are subjected to a high immune burden more frequently than SIRS patients without an infection. In the following steps it is shown that the score calculated from the expression of those markers in accordance with formula 1 depicts the host response in a similar way as the master score introduced in the 1^{st} application example. Moreover, it is shown that by extending the marker set, a deviation from the master score can be reduced very clearly. In the gene selection of the 1^{st} application example 6 gene sequences from applicant's non-pre-published application (DE 10 2009 044 085) are to be found that are indicated herein by their symbols: TLR5 and CD59 in heat map cluster 2, CPVL and FGL2 in heat map cluster 5, HLA-DPA1 and IL7R in heat map cluster 6. For a further gene marker (CLU) the gene probe used on the micro array BeadChip HumanHT-12 v3 did not provide any signals. For these 7 gene markers, expression values for the 67 samples of patients from table 2 were provided that were measured on an alternative platform, the so-called real time PCR. From the list of 8537 gene probes the replacement TFPI for CLU was selected, the expression values of which on the micro array together with the expression values on the real time PCR corresponding to marker CLU, reached a correlation of 0.8 (correlation co-efficient according to Pearson).

From the gene expression of the 73 examined RNA samples that was determined on the micro array for the 7 tuple of gene probes, we calculated the score in accordance with formula 1. Its deviation from the master score amounted to a maximum of ±6 percentage points for half of the samples, a maximum of ±11 percentage points for three quarters of the samples, and ±18.3 percentage points for 90% of the samples.

In a next step, 1 gene probe of the remaining 8530 gene probes each was successively added to the set of 7 markers and the corresponding score was calculated in accordance with formula 1. The set was extended by the probe that provided the minimal error. The error was defined as the 95% quantile of all absolute deviations from the master score. The procedure was repeated until the error amounted to less than ±10 percentage points. This occurred after the gene marker tuple was extended to 10 gene probes. The value of the score for 7 and 10 gene probes as well as the master score were summarized in Table 5. The table further indicates the with regard to basis 2 logarithmized expression signals of the corresponding gene sequences.

TABLE 5
Score values for 7 and 10 selected gene probes as compared to the master score (columns 2 to 4).
Expression values logarithmized for basis 2 for 10 selected gene probes that are characterized
by the corresponding sequence number (2nd line), accession (3rd line) and symbol (4th line). The
5th line indicates the heat map cluster into which the corresponding probe was classified.

| | | | | Expression signals of gene sequences logarithmized for basis 2 | | | |
|---|---|---|---|---|---|---|---|
| | | | | 1983 | 507 | 5431 | 3043 |
| | Score | | | NM_003268 | NM_000611 | NM_031311 | NM_006682 |
| Sample | 7 Gene | 10 Gene | | TLR5 | CD59 | CPVL | FGL2 |
| ID | probes | probes | Master | (2) | (2) | (5) | (5) |
| 1 | 1.8 | 2.8 | −4.7 | 10.1 | 7.5 | 11.6 | 13.2 |
| 2 | 5.3 | 1.4 | 10.0 | 10.8 | 7.8 | 13.2 | 14.0 |
| 3 | 0.2 | −1.6 | −9.3 | 10.2 | 7.3 | 12.3 | 12.9 |
| 4 | 3.3 | 8.3 | 11.1 | 10.1 | 7.7 | 13.0 | 13.8 |
| 5 | −7.3 | −12.6 | −7.3 | 9.5 | 7.3 | 12.6 | 13.1 |
| 6 | −6.3 | 0.2 | −8.2 | 10.3 | 7.3 | 12.8 | 13.4 |
| 7 | −5.7 | −6.8 | −5.2 | 9.9 | 7.5 | 12.6 | 13.3 |
| 8 | 3.2 | 3.0 | 10.7 | 10.3 | 7.2 | 12.8 | 13.8 |
| 9 | −2.1 | −2.8 | −3.2 | 10.2 | 7.3 | 13.0 | 12.9 |
| 10 | 8.6 | 6.8 | 7.5 | 10.7 | 7.5 | 13.3 | 13.6 |
| 11 | 11.4 | 12.2 | 17.2 | 11.0 | 7.3 | 13.2 | 13.8 |
| 12 | 12.4 | 15.2 | 5.2 | 11.0 | 7.5 | 13.6 | 13.8 |
| 13 | 20.5 | 26.3 | 35.2 | 11.6 | 7.7 | 13.4 | 14.3 |
| 14 | 25.7 | 24.8 | 19.7 | 11.6 | 8.0 | 13.2 | 14.1 |
| 15 | 16.7 | 16.2 | 20.8 | 11.3 | 7.5 | 13.1 | 13.8 |
| 16 | 30.5 | 28.6 | 33.2 | 12.2 | 8.5 | 13.4 | 14.1 |
| 17 | 15.4 | 18.6 | 9.5 | 11.7 | 7.6 | 12.8 | 13.6 |
| 18 | 23.7 | 19.4 | 26.1 | 11.7 | 7.9 | 12.7 | 13.8 |
| 19 | 18.8 | 22.2 | 15.8 | 11.4 | 7.5 | 13.4 | 13.6 |
| 20 | 0.0 | 0.9 | 2.4 | 10.4 | 7.3 | 12.9 | 13.5 |
| 21 | 5.2 | 12.9 | 8.5 | 10.8 | 7.6 | 13.2 | 13.6 |
| 22 | −4.3 | 5.2 | 6.3 | 10.3 | 7.5 | 13.0 | 14.1 |
| 23 | 14.9 | 10.1 | 5.4 | 11.2 | 7.7 | 13.0 | 13.3 |
| 24 | 14.2 | 11.8 | 19.6 | 11.2 | 7.5 | 13.0 | 13.8 |
| 25 | 47.2 | 40.4 | 41.5 | 12.2 | 8.1 | 12.5 | 13.4 |
| 26 | 31.5 | 41.6 | 39.7 | 12.1 | 7.7 | 13.1 | 13.9 |
| 27 | 40.3 | 46.4 | 40.6 | 12.8 | 8.8 | 13.8 | 13.6 |
| 28 | 40.0 | 43.1 | 42.8 | 12.3 | 8.2 | 12.5 | 13.4 |
| 29 | 38.4 | 37.4 | 33.4 | 12.5 | 8.0 | 13.4 | 13.5 |
| 30 | 0.2 | 0.6 | 6.8 | 10.1 | 7.5 | 12.7 | 13.7 |
| 31 | 23.7 | 29.6 | 50.1 | 10.3 | 7.3 | 13.1 | 13.6 |
| 32 | 51.9 | 63.1 | 57.1 | 11.9 | 8.7 | 11.2 | 13.2 |
| 33 | 6.7 | 25.4 | 33.7 | 10.5 | 7.8 | 12.7 | 13.8 |
| 34 | 1.3 | 5.3 | 2.6 | 10.5 | 7.3 | 13.1 | 13.9 |
| 35 | 73.6 | 79.6 | 76.9 | 12.6 | 8.9 | 12.1 | 12.2 |
| 36 | 75.6 | 87.1 | 85.4 | 13.3 | 9.6 | 12.7 | 12.9 |
| 37 | 46.3 | 47.3 | 27.7 | 12.6 | 7.6 | 12.5 | 12.0 |
| 38 | 47.4 | 52.9 | 49.1 | 12.7 | 7.9 | 13.2 | 12.9 |
| 39 | 32.6 | 40.5 | 31.0 | 12.0 | 7.7 | 12.8 | 13.2 |
| 40 | 45.4 | 61.5 | 62.6 | 12.8 | 7.9 | 13.6 | 13.4 |
| 41 | 46.9 | 57.2 | 61.3 | 12.2 | 7.6 | 12.7 | 13.1 |
| 42 | 62.6 | 68.6 | 71.5 | 13.0 | 8.3 | 12.1 | 12.3 |
| 43 | 99.6 | 93.0 | 90.4 | 13.3 | 8.6 | 11.3 | 12.2 |
| 44 | 78.8 | 75.8 | 83.0 | 12.9 | 8.0 | 12.5 | 13.9 |
| 45 | 93.3 | 93.6 | 86.4 | 12.4 | 7.7 | 11.6 | 11.7 |
| 46 | 69.4 | 77.6 | 71.9 | 12.7 | 9.1 | 11.5 | 12.3 |
| 47 | 60.1 | 74.4 | 65.6 | 12.7 | 7.7 | 11.8 | 12.2 |
| 48 | 85.7 | 93.0 | 93.0 | 13.4 | 8.7 | 10.8 | 11.9 |
| 49 | 101.6 | 95.2 | 92.5 | 13.1 | 8.3 | 9.2 | 13.5 |
| 50 | 60.8 | 63.8 | 66.1 | 12.9 | 7.9 | 11.9 | 13.3 |
| 51 | 69.3 | 83.7 | 80.5 | 13.1 | 8.6 | 12.6 | 13.1 |
| 52 | 86.8 | 85.2 | 80.0 | 13.1 | 9.2 | 11.5 | 12.2 |
| 53 | 79.9 | 74.2 | 78.4 | 13.1 | 7.9 | 11.4 | 12.1 |
| 54 | 67.0 | 77.6 | 72.8 | 12.4 | 8.7 | 12.8 | 13.0 |
| 55 | 35.3 | 38.2 | 38.0 | 11.9 | 7.8 | 13.2 | 13.0 |
| 56 | 120.1 | 110.7 | 97.2 | 12.6 | 8.4 | 9.9 | 13.3 |
| 57 | 99.3 | 106.0 | 106.2 | 13.5 | 9.1 | 12.0 | 12.3 |
| 58 | 78.1 | 90.5 | 94.3 | 11.8 | 9.1 | 12.4 | 11.0 |
| 59 | 151.0 | 131.4 | 127.8 | 13.9 | 8.7 | 8.6 | 11.5 |
| 60 | 98.3 | 105.4 | 115.2 | 13.7 | 8.6 | 10.6 | 12.2 |
| 61 | 62.0 | 65.0 | 60.3 | 12.6 | 8.5 | 12.4 | 13.8 |
| 1_t0 | 0.9 | 20.1 | 22.4 | 10.7 | 7.9 | 13.5 | 14.4 |
| 1_t1 | 65.7 | 85.7 | 80.8 | 13.0 | 8.8 | 12.4 | 13.0 |
| 1_t2 | 78.4 | 95.6 | 95.3 | 13.2 | 9.1 | 12.5 | 13.1 |
| 1_t3 | 73.2 | 84.0 | 86.7 | 12.8 | 8.6 | 12.0 | 12.8 |
| 1_t4 | 86.6 | 95.2 | 94.2 | 12.9 | 8.5 | 11.5 | 12.9 |
| 1_t5 | 65.3 | 85.1 | 92.8 | 12.8 | 8.0 | 12.0 | 13.4 |
| 2_t0 | 13.1 | 24.7 | 33.8 | 10.3 | 7.6 | 13.3 | 13.8 |
| 2_t1 | 77.9 | 84.7 | 82.8 | 12.6 | 7.9 | 12.1 | 12.9 |
| 2_t2 | 82.3 | 88.4 | 83.4 | 13.3 | 8.6 | 12.8 | 13.4 |
| 2_t3 | 104.7 | 94.2 | 90.3 | 12.7 | 8.8 | 11.0 | 12.5 |
| 2_t4 | 56.8 | 53.3 | 56.7 | 12.2 | 7.8 | 12.7 | 13.6 |
| 2_t5 | 57.5 | 52.7 | 59.4 | 11.5 | 7.7 | 12.9 | 13.5 |

| | Expression signals of gene sequences logarithmized for basis 2 | | | | | |
|---|---|---|---|---|---|---|
| | 1665 | 5776 | 2902 | 6585 | 3167 | 745 |
| | NM_002185 | NM_033554 | NM_006287 | NM_174918 | NM_007111 | NM_001007535 |
| Sample | IL7R | HLA-DPA1 | TFPI | C19orf59 | TFDP1 | dJ341D10.1 |
| ID | (6) | (6) | (4) | (2) | (4) | (3) |
| 1 | 13.8 | 13.3 | 7.1 | 10.9 | 10.4 | 8.5 |
| 2 | 13.1 | 14.1 | 6.9 | 11.3 | 10.1 | 8.3 |
| 3 | 13.2 | 13.9 | 7.0 | 10.8 | 9.4 | 8.0 |
| 4 | 12.9 | 13.6 | 7.0 | 11.3 | 11.1 | 9.4 |
| 5 | 12.9 | 14.0 | 6.9 | 9.7 | 10.1 | 8.3 |
| 6 | 13.5 | 14.0 | 6.7 | 11.4 | 10.4 | 8.6 |
| 7 | 13.5 | 13.9 | 6.9 | 10.5 | 10.0 | 8.4 |
| 8 | 12.8 | 14.0 | 7.3 | 11.5 | 9.6 | 8.1 |
| 9 | 13.4 | 13.8 | 7.0 | 10.7 | 10.2 | 8.3 |
| 10 | 12.8 | 13.8 | 7.1 | 11.3 | 10.0 | 8.8 |
| 11 | 12.4 | 14.0 | 7.0 | 11.9 | 10.1 | 8.6 |
| 12 | 13.0 | 13.1 | 7.2 | 11.9 | 10.8 | 8.6 |
| 13 | 12.7 | 13.4 | 7.0 | 12.5 | 11.7 | 8.8 |
| 14 | 13.4 | 13.5 | 7.6 | 12.3 | 10.6 | 9.0 |
| 15 | 12.9 | 13.8 | 7.3 | 11.6 | 10.5 | 9.3 |
| 16 | 13.2 | 13.7 | 7.1 | 12.5 | 10.5 | 9.5 |
| 17 | 13.3 | 13.7 | 6.8 | 12.3 | 10.5 | 8.6 |
| 18 | 12.8 | 13.0 | 6.7 | 11.9 | 9.8 | 8.9 |
| 19 | 13.5 | 13.9 | 8.1 | 12.5 | 10.6 | 8.9 |
| 20 | 12.9 | 14.0 | 6.7 | 10.9 | 10.7 | 8.4 |
| 21 | 12.9 | 14.0 | 6.8 | 12.1 | 10.3 | 9.0 |
| 22 | 13.2 | 14.2 | 6.9 | 11.7 | 10.7 | 9.0 |
| 23 | 13.3 | 13.7 | 7.3 | 11.3 | 10.3 | 8.7 |
| 24 | 13.1 | 13.5 | 7.2 | 11.8 | 10.3 | 8.3 |
| 25 | 11.6 | 12.9 | 7.7 | 13.0 | 9.9 | 8.7 |
| 26 | 12.2 | 13.4 | 7.1 | 14.1 | 10.5 | 8.9 |
| 27 | 12.3 | 13.8 | 6.8 | 14.4 | 10.3 | 9.1 |
| 28 | 12.6 | 12.7 | 7.3 | 13.7 | 10.6 | 8.6 |
| 29 | 12.3 | 13.2 | 7.3 | 13.5 | 9.9 | 8.7 |
| 30 | 13.5 | 13.5 | 7.2 | 11.0 | 10.6 | 8.3 |
| 31 | 11.6 | 12.8 | 7.6 | 12.2 | 12.7 | 8.8 |
| 32 | 11.5 | 12.7 | 7.0 | 14.6 | 11.8 | 9.4 |
| 33 | 12.8 | 13.6 | 6.6 | 12.7 | 11.6 | 9.8 |
| 34 | 13.2 | 13.7 | 7.0 | 11.8 | 9.9 | 8.5 |
| 35 | 11.6 | 11.7 | 9.0 | 14.9 | 11.8 | 10.2 |
| 36 | 11.2 | 11.5 | 7.3 | 15.8 | 11.0 | 10.8 |
| 37 | 12.3 | 12.4 | 7.2 | 14.4 | 9.6 | 7.4 |
| 38 | 11.6 | 13.0 | 7.5 | 14.7 | 9.9 | 8.7 |
| 39 | 12.6 | 13.0 | 7.5 | 13.1 | 12.0 | 9.0 |
| 40 | 12.7 | 12.4 | 7.9 | 14.9 | 11.9 | 9.4 |
| 41 | 12.5 | 12.3 | 8.7 | 14.2 | 12.4 | 9.0 |
| 42 | 12.0 | 12.3 | 8.2 | 15.1 | 11.3 | 8.7 |
| 43 | 10.3 | 11.2 | 9.5 | 15.2 | 11.6 | 9.5 |
| 44 | 10.4 | 10.5 | 7.7 | 14.5 | 10.2 | 10.3 |
| 45 | 9.6 | 10.9 | 7.9 | 15.6 | 12.8 | 8.9 |
| 46 | 11.2 | 12.1 | 7.0 | 15.3 | 11.4 | 9.6 |
| 47 | 12.0 | 12.4 | 8.6 | 15.4 | 11.0 | 10.2 |
| 48 | 11.3 | 11.1 | 7.7 | 15.9 | 11.3 | 10.5 |
| 49 | 10.5 | 10.2 | 7.9 | 14.7 | 12.6 | 10.0 |
| 50 | 12.0 | 12.2 | 8.4 | 14.8 | 11.0 | 8.7 |
| 51 | 11.6 | 12.5 | 8.8 | 15.6 | 12.9 | 9.6 |
| 52 | 11.0 | 10.7 | 7.5 | 15.1 | 10.5 | 10.3 |
| 53 | 10.9 | 11.9 | 8.7 | 14.4 | 11.2 | 9.1 |
| 54 | 10.6 | 12.1 | 7.9 | 15.6 | 11.1 | 9.7 |
| 55 | 12.9 | 12.9 | 8.4 | 13.1 | 11.4 | 8.7 |
| 56 | 8.0 | 10.6 | 8.5 | 15.7 | 11.6 | 9.9 |
| 57 | 9.6 | 11.7 | 8.7 | 16.0 | 12.1 | 11.2 |
| 58 | 10.7 | 11.2 | 8.6 | 15.5 | 12.0 | 11.2 |
| 59 | 8.0 | 9.5 | 7.7 | 16.2 | 10.6 | 9.9 |
| 60 | 10.6 | 11.7 | 9.0 | 16.0 | 12.7 | 10.7 |
| 61 | 10.9 | 12.7 | 8.0 | 14.5 | 11.4 | 9.0 |
| 1_t0 | 13.0 | 14.3 | 6.8 | 12.8 | 11.0 | 9.9 |
| 1_t1 | 11.0 | 12.8 | 7.6 | 15.8 | 11.9 | 10.9 |
| 1_t2 | 10.8 | 11.9 | 8.0 | 16.0 | 12.0 | 11.5 |
| 1_t3 | 11.2 | 11.7 | 8.4 | 15.4 | 12.5 | 9.9 |
| 1_t4 | 10.1 | 11.8 | 8.6 | 15.7 | 13.3 | 9.6 |
| 1_t5 | 11.3 | 12.2 | 8.3 | 15.8 | 12.8 | 10.2 |
| 2_t0 | 12.0 | 14.3 | 7.9 | 12.3 | 12.5 | 9.4 |
| 2_t1 | 9.6 | 12.4 | 8.4 | 15.4 | 11.8 | 9.9 |
| 2_t2 | 9.5 | 12.6 | 8.0 | 15.7 | 11.9 | 9.6 |
| 2_t3 | 9.0 | 11.0 | 8.2 | 14.1 | 12.0 | 11.3 |
| 2_t4 | 10.2 | 13.3 | 8.0 | 13.3 | 11.2 | 9.3 |
| 2_t5 | 9.9 | 12.8 | 8.4 | 13.2 | 11.2 | 9.0 |

### Example 3

### Determining the Severity of a Host Response on Alternative Platforms

As was already mentioned in the 2nd example, gene expression signals were measured by way of a real time PCR for 7 relevant gene markers from table 5 and for 67 RNA samples. For this, the following measuring and analyzing steps were performed.

### Real-Time-PCR

The Platinum SYBR Green q PCR SuperMix-UDG kit of Invitrogen (Invitrogen Germany, Karlsruhe, Federal Republic of Germany) was used. The cDNA of the patients was diluted with water in a ratio of 1:25, of which 1 μl was utilized in the PCR. Each of the samples was replicated 3 times using a pipette.

PCR formulation per well (10 μl) 2 μl of template cDNA 1:100
1 μl of forward primer, 10 mM
1 μl of reverse primer, 10 mM
1 μl of Fluorescin Reference Dye
5 μl of Platinum SYBR Green qPCR SuperMix-UDG

A master mix without a template was manufactured which was aliquoted in the PCR plate in 9 μl aliquots; for this, the patients' cDNAs, respectively, were pipetted.

The subsequent PCR program consisted of the following steps:

| | |
|---|---|
| 95° C. | 2 min (activation of polymerase) |
| 95° C. | 10 sec (denaturation)* |
| 58° C. | 15 sec (apposition)* |
| 72° C. | 20 sec (extension)* |
| 55° C.-95° C. | 10 sec (generation of a melting graph, ** |
| | increase of the initial temperature |
| | after each step by 1° C.) |

*40 x
** 41 x

The iQ™ 5 Multicolor Real-Time-PCR Detection System of BIORAD with the corresponding evaluation software was used. The so-called Ct values (estimated number of cycles in exceeding a threshold) were calculated by the program automatically as measuring result in the range of a linear increase of the curve. The measuring values were stored in the string format.

### Data Analysis:

The data analysis was performed using the free software R Project version R 2.8.0 (R.app GUI 1.26 (5256), S. Urbanek & S. M. lacus, © R Foundation for Statistical Computing, 2008), which is available under www.r-project.org (cf. R Development Core Team, 2006).

The data matrices of the measured Ct values that were used in the analysis, were processed as follows. Together with the marker genes 3 so-called housekeeper genes were measured that were used as references. For normalizing, an average value of the 3 selected housekeeper genes was computed for each sample. From this value, the Ct value of each individual marker was deducted. Each Delta Ct value thus gained reflects the relative abundance of the target transcript related to the calibrator, a positive Delta Ct value signifying one abundance higher than an average value of the references, and a negative Delta Ct value signifying one abundance lower than an average value of the references.

The data mix of normalized Delta Ct values was summarized in table 6. In accordance with formula 1 and the description in the 1^{st} example, the corresponding score for quantifying the severity of a host response was computed from the expression signals that were measured by way of real time PCR. A comparison of this score with the master score is shown in FIG. 6, the master score being depicted by a corresponding error bar of 10 percentage points upwards and downwards, and the score determined from the PCR measurement being depicted as rhombus. As was already described in the 1^{st} example, the calculation provision of the score is independent of the number of gene markers used and of the measuring platform; for calculating the score, merely the average expression signals of phenotype groups NaN and SaS, which were defined in table 1, are required. As is apparent from FIG. 6, the score, which was determined by real time PCR measurement of 7 markers, exhibits a similar trend as the master score and thus points out to the severity of the host response under an acute inflammatory burden of the organism. It should be mentioned that real time PCR is a simpler, quicker and less expensive measuring platform for determining gene expression than a micro array. Its constraint is the lower number of markers measurable concurrently.

TABLE 6
Summary of the Delta Ct values normalized with regard to 3 references for 7 selected gene sequences
and 67 RNA samples. The sample ID relates to samples of the patients that were depicted in table 2.

| | Sequence-IDs | | | | | | |
|---|---|---|---|---|---|---|---|
| | | 507, 7695, | | | | | |
| | | 7696, 7697, | | | | | |
| | | 7698, 7699, | | | | | 7702, |
| | 1983 | 7700, 7701 | 5431, 4636 | 3043 | 1665 | 5776 | 7703, 7704 |
| | Symbol | | | | | | |
| | TLR5 | CD59 | CPVL | FGL2 | IL7R | HLA-DPA1 | CLU |
| backward | Primer sequence: forward | | | | | | |
| Sample ID | 7717 7718 | 7705 7706 | 7709 7710 | 7711 7712 | 7715 7716 | 7713 7714 | 7707 7708 |
| 1 | −2.25 | −1.84 | −0.19 | 1.14 | −1.33 | −1.50 | 0.01 |
| 2 | −4.07 | −2.43 | −0.62 | 1.53 | −0.18 | 2.41 | −1.59 |
| 3 | −6.00 | −2.30 | −1.01 | 0.87 | −0.44 | 2.85 | −1.29 |
| 4 | −5.53 | −2.87 | −0.32 | 1.84 | −0.65 | 2.12 | −0.43 |
| 6 | −5.11 | −3.84 | −0.97 | 0.67 | −1.12 | 2.19 | −1.26 |
| 7 | −6.00 | −2.70 | −0.74 | 1.18 | −0.47 | 2.46 | −3.19 |
| 9 | −5.17 | −2.00 | 0.06 | 1.58 | −0.03 | 2.49 | −0.94 |
| 10 | −4.54 | −2.54 | 0.45 | 1.87 | −0.80 | 1.94 | −0.38 |
| 11 | −3.66 | −2.07 | 0.00 | 2.20 | −1.48 | 2.55 | −1.04 |
| 12 | −3.85 | −2.41 | 0.49 | 1.14 | −0.52 | −1.41 | 0.13 |
| 13 | −2.62 | −1.92 | 0.59 | 2.77 | −1.36 | 1.47 | −0.48 |
| 14 | −2.65 | −1.73 | −0.08 | 1.59 | −1.06 | 1.53 | −0.03 |
| 15 | −4.17 | −2.43 | −0.24 | 1.22 | −1.08 | 2.15 | 0.56 |
| 16 | −2.84 | −1.89 | −0.68 | 1.47 | −2.00 | 2.15 | −0.31 |
| 17 | −3.57 | −2.58 | 0.00 | 1.94 | −0.53 | 2.09 | −1.00 |
| 18 | −2.82 | −2.15 | −0.61 | 1.02 | −1.05 | 0.97 | −0.69 |
| 21 | −5.03 | −2.02 | −0.29 | 1.30 | 0.06 | 2.31 | −0.90 |
| 22 | −4.66 | −2.56 | −0.53 | 1.77 | −0.46 | 2.52 | −1.58 |
| 23 | −4.49 | −2.83 | −1.05 | 0.88 | −1.38 | 2.46 | 0.18 |
| 24 | −3.68 | −2.79 | −0.55 | 1.57 | −0.67 | 2.04 | −1.22 |
| 25 | −5.12 | −1.60 | −0.04 | 1.68 | −1.53 | 1.17 | 0.93 |
| 26 | −2.89 | −1.75 | 0.11 | 2.35 | −2.04 | 1.71 | −0.23 |
| 27 | −1.61 | −0.87 | 0.15 | 1.26 | −1.27 | 1.15 | 0.10 |
| 28 | −5.16 | −1.62 | −0.26 | 1.26 | −1.01 | 1.08 | −0.24 |
| 29 | −2.63 | −2.23 | 0.49 | 1.77 | −1.22 | 0.75 | 0.76 |
| 30 | −5.35 | −2.54 | −0.83 | 1.62 | −0.98 | 2.42 | −0.21 |
| 31 | −4.71 | −2.30 | 0.23 | 2.13 | −2.87 | 2.14 | 1.16 |
| 32 | −4.07 | −0.74 | −1.33 | 1.66 | −1.79 | 1.84 | −0.34 |
| 33 | −4.12 | −2.46 | −0.56 | 1.77 | −0.40 | 1.53 | −0.37 |
| 34 | −5.24 | −3.25 | −0.25 | 1.38 | −0.05 | 0.86 | −1.17 |
| 35 | −1.59 | 0.20 | −0.94 | 1.07 | −2.55 | −0.44 | 2.73 |
| 36 | −0.43 | 0.74 | −0.73 | 0.49 | −3.11 | −0.77 | −0.27 |
| 37 | −2.15 | −2.19 | −0.22 | 1.36 | −1.80 | 2.00 | 0.57 |
| 40 | −1.32 | −1.13 | 0.61 | 1.47 | −2.70 | 0.96 | 1.71 |
| 41 | −5.74 | −1.33 | −0.50 | 0.88 | −2.97 | 0.61 | 1.25 |
| 42 | −2.07 | −0.67 | −1.92 | −0.11 | −3.74 | −0.09 | 1.01 |
| 43 | −1.61 | −0.66 | −2.33 | 0.06 | −5.89 | −0.32 | 2.98 |
| 44 | −1.12 | −1.14 | −1.18 | 1.60 | −3.10 | −1.50 | 0.86 |
| 45 | −4.55 | −0.58 | −1.26 | −0.36 | −1.22 | −0.54 | 0.89 |
| 46 | −1.81 | 0.49 | −1.78 | 0.41 | −1.95 | 1.31 | 0.26 |
| 47 | −4.57 | −1.01 | −1.51 | 0.09 | −2.03 | 0.86 | 0.37 |
| 48 | −1.95 | 0.51 | −4.10 | −1.18 | −3.53 | −0.60 | 0.23 |
| 49 | −6.00 | −0.58 | −5.08 | 1.61 | −3.96 | −1.15 | 0.62 |
| 50 | −4.77 | −1.53 | −1.65 | 0.56 | −2.74 | 0.24 | 1.91 |
| 51 | −0.66 | −0.40 | −0.69 | 0.65 | −3.07 | 0.27 | 2.06 |
| 52 | −1.53 | 0.70 | −1.82 | 0.38 | −2.45 | −0.79 | 0.87 |
| 53 | −3.70 | −0.65 | −1.85 | 0.69 | −2.93 | 0.35 | 1.92 |
| 54 | −1.64 | 0.40 | −0.27 | 1.05 | −2.89 | 0.92 | 1.61 |
| 55 | −5.13 | −1.34 | 0.20 | 0.88 | −0.68 | 1.70 | 1.85 |
| 56 | −1.13 | 0.47 | −2.00 | 1.20 | −3.25 | 0.03 | 1.44 |
| 57 | −1.54 | 0.77 | −1.23 | −0.16 | −4.08 | 0.15 | 2.15 |
| 58 | −1.53 | 1.35 | −0.03 | 0.11 | −1.94 | −0.08 | 2.23 |
| 59 | 0.15 | 1.01 | −4.02 | −1.40 | −4.17 | −1.50 | 1.06 |
| 60 | −0.18 | 0.51 | −3.53 | −0.24 | −5.21 | −0.63 | 2.41 |
| 61 | −2.23 | −0.74 | −0.62 | 1.66 | −3.92 | 0.92 | 1.03 |
| 1_t0 | −5.06 | −2.18 | −0.25 | 2.04 | −0.15 | 2.46 | −1.88 |
| 1_t1 | −1.61 | 0.06 | −0.86 | 0.75 | −2.41 | 1.23 | 0.98 |
| 1_t2 | −1.91 | 0.09 | −1.61 | 0.15 | −3.13 | 0.17 | 1.20 |
| 1_t3 | −1.87 | 0.60 | −1.27 | 0.85 | −2.96 | 1.02 | 2.22 |
| 1_t4 | −2.50 | 0.75 | −2.16 | 0.85 | −4.17 | 0.85 | 1.87 |
| 1_t5 | −2.09 | 0.11 | −1.94 | 0.95 | −3.28 | 0.65 | 1.92 |
| 2_t0 | −6.00 | −1.30 | 0.71 | 2.09 | −0.85 | 3.32 | 2.22 |
| 2_t1 | −4.17 | −0.35 | −0.83 | 0.99 | −3.37 | 0.46 | 2.15 |
| 2_t2 | −3.12 | −0.57 | −0.42 | 1.40 | −3.80 | 0.58 | 2.21 |
| 2_t3 | −4.70 | −0.66 | −2.42 | 0.39 | −3.65 | −0.68 | 1.75 |
| 2_t4 | −4.65 | −1.53 | 0.23 | 1.92 | −2.69 | 1.20 | 1.82 |
| 2_t5 | −5.62 | −1.56 | −0.15 | 1.79 | −3.43 | 0.65 | 1.32 |

### Example 4

### Differential Expression of Proteins for In Vitro Determination of the Severity of a Host Response of Patients

The differential expression of markers for in-vitro determination of the severity of a host response of patients cannot only be effected by means of transcriptomic markers, but also on a protein level. There are numerous examples for the use of proteins as bio markers, which was already addressed shortly (Pierrakos, 2010). Likewise, it is pointed out to the fact that individual protein markers so far do not provide satisfactory results or provide merely mediocre results and that combinations of protein markers preferably should be applied.

As an example, experiments are described in the following the result of which proves that protein markers are equally suited for in vitro determination of the severity of a host response of patients. Preferably those proteins were selected for examination for the gene transcripts of which it could already be shown in previous examples that they are suited for the purpose indicated.

### Examined Group of Patients

Samples of 9 patients with sepsis, 3 patients after cardiac surgery interventions and 7 healthy test persons were examined (EDTA blood samples). The patients that had undergone a cardiac surgery intervention (ICU patients) according to clinical criteria were classified with the diagnosis of SIRS (Systemic Inflammatory Response Syndrome) at the time the samples were taken. The three groups of patients represent the phenotypes LaS and SaS (together), NaS and NaN form table 1.

### Experimental Implementation

From the blood samples two cell populations of white blood cells were isolated with the aid of a density-gradient (Lymphocyte Separation Medium LSM 1077, PAA Laboratories GmbH, Colbe): peripheral mononuclear cells (peripheral blood mononuclear cells, PBMCs) and polymorphonuclear cells (polymorphonuclear cells, PMNs). The cells used for the experiments represented two sub-populations of the PBMCs: the T lymphocytes and the monocytes.

Detection of the proteins was done by way of flow cytometry and Western Blot, flow cytometry being used for surface proteins, and Western Blot being used for intracellular proteins. For both methods monoclonal antibodies were preferably used.

By way of flow cytometry (FACSCalibur Flow Cytometer, Becton Dickinson GmbH Heidelberg) the expression of proteins of the following genes was examined for T lymphocytes and monocytes: CD59, HLA-DPA1, IL7R, TLR5, and HLA-DR complex. The analyzed blood samples were composed as follows: sepsis patients (n=9), ICU patients (n=3) and healthy controls (n=7). T lymphocytes and monocytes were distinguished with the aid of two surface markers: CD3 coreceptor for T lymphocytes [Tsoukas et al., 1985], and CD14 receptor for monocytes [Goyert et al., 1988]. Moreover, the cells were identified by means of their cell size (FSC-H) and their granularity (SSC-H).

By way of Western Blot the expression of the proteins from the following genes was examined in the lysate of the total population of PBMCs in sepsis patients and healthy test persons: FGL2, CLU and CPVL. The experiments were carried out using Standard Western Blot listings. Positive controls (lysates of transfected cells) were always performed and normalization of the experiments was done on the basis of β actin. The anti-fibrinogen-like protein 2 (product of FGL2), anti-clusterin (product of CLU gene) and anti-vitellogenic carboxypeptidase-like protein (product of CPVL gene) antibodies were monoclonal mouse antibodies, the secondary antibody was a HRP-coupled rabbit-anti-mouse-antibody. The antibody for β actin was a monoclonal (13E5) rabbit antibody (Cell Signalling Technology Inc., Danvers, USA).

### Data Analysis

The measuring data were provided by the software of the respective measuring device. They are summarized in table 7. The expression of individual proteins for the 2 to 3 examined groups of phenotypes was tested for statistically significant differences. In so doing, as in the 1^{st} embodiment, the one-way analysis of variance (Anova) and the pairwise t-test were used. The results of the comparisons are listed at the end of table 7. They are summarized in the following.

In T lymphcytes the protein expression from the IL7R gene was significantly lower in sepsis patients than in healthy donors. Thus, a change in the same direction as in the gene expression was to be noted. The expression of MHC class II HLA-DPA1 antigen (product from HLA-DPA1 gene) was significantly higher in sepsis patients than in healthy donors. Thus, a change in the contrary direction than in gene expression was to be noted. In the protein expression from CD59, TLR5 and HLA-DR genes the T lymphocytes exhibited no significant differences between the examined groups of phenotypes. In the protein expression from genes HLA-DPA1 and TLR5 the monocytes did not exhibit any differences in the 3 groups: the protein expression from the CD59 and HLA-DR gene, however, was significantly higher in healthy test persons than in SIRS and sepsis patients. An IL7R gene product could not be traced in monocytes.

The Western-Blot analysis of PBMC lysates revealed that the protein expression from the FGL2 gene is significantly increased in sepsis patients in comparison to healthy controls, whereas the protein expression from the CLU gene is reduced in sepsis patients. Thus, for both proteins a change in the expression in a contrary direction than in gene expression was revealed. A CPVL gene product could not be traced in the lysates.

TABLE 7
Protein expression values for selected markers. Non-measured values were referred to by n.a.
Average values of patients that were significantly different from those of healthy persons,
were marked correspondingly (“**” for p <0.01, “*” for p <0.05, und “⁺” for p <0.1).

| | | Protein from gene | | | | | | |
|---|---|---|---|---|---|---|---|---|
| | | TLR5 | | CD59 | | CPVL | FGL2 | IL7R |
| | | Cell type | | | | | | |
| | | T-Lympho- | Mono- | T-Lympho- | Mono- | Whole blood | Whole blood | T-Lympho- |
| | | cytes | cytes | cytes | cytes | [Ratio for β | [Ratio for β | cytes |
| Sample number | | [%] | [%] | [%] | [%] | Actin] | Actin]] | [%] |
| Donor | 1 | 85,.2 | n.a. | 76.1 | 86.9 | 0 | 0.41 | 28.7 |
| | 2 | 39.8 | 73.2 | 78.8 | 77.1 | 0 | 0.52 | 33 |
| | 3 | 58.9 | 87.1 | 86.5 | 88.4 | n.a. | n.a. | 49.3 |
| | 4 | 62.7 | n.a. | 72.7 | 75.4 | 0 | 1.23 | 30.8 |
| | 5 | 54 | 91.5 | 84.2 | 63.5 | 0 | 0.87 | 39.6 |
| | 6 | 74.6 | 64.3 | 64.8 | 65 | n.a. | n.a. | 24.3 |
| | 7 | 75.8 | 81 | 81.7 | 76.4 | n.a. | n.a. | 43.3 |
| SIRS | 8 | 38.9 | 60.6 | 69.3 | 82 | n.a. | n.a. | 32.7 |
| | 9 | 77.2 | 80.9 | 64 | 58.9 | n.a. | n.a. | 20.7 |
| | 10 | 35.7 | 75.8 | 44.6 | 84.1 | n.a. | n.a. | 8.3 |
| severe | 11 | 60.3 | n.a. | 63.9 | 0 | n.a. | n.a. | 17 |
| sepsis/ | 12 | 89.3 | 54.9 | 80.7 | 37.6 | n.a. | n.a. | 19.7 |
| septic | 13 | 35.2 | 20.5 | 93.3 | 23.2 | 0 | 1.01 | 10.3 |
| shock | 14 | 80.2 | 93.7 | 68 | 73.3 | 0 | 1.34 | 18.9 |
| | 15 | 87.9 | 53.5 | 93.8 | 26.3 | n.a. | n.a. | 4.9 |
| | 16 | 68.8 | n.a. | 78.7 | 8.5 | n.a. | n.a. | 15 |
| | 17 | 51.8 | 68.1 | 79.9 | 47.7 | n.a. | n.a. | 9.4 |
| | 18 | 71.1 | 72.9 | 83.8 | 51.3 | 0 | 1.68 | 23.6 |
| | 19 | n.a. | n.a. | 86.8 | 77.2 | n.a. | n.a. | 33.2 |
| Average | Donor | 64.4 | 79.4 | 77.8 | 76.1 | 0 | 0.76 | 35.6 |
| value | SIRS | 50.6 | 72.4 | 59.3⁺ | 75.0 | n.a. | n.a. | 20.6 |
| | Sepsis | 68.1 | 60.6 | 81 | 38.3** | 0 | 1.34⁺ | 16.9** |
| P value | Anova | 0.381 | 0.270 | 0.013 | 0.004 | n.a. | 0.084 | 0.003 |
| | SIRS vs. | 0.416 | 0.416 | 0.118 | 0.909 | n.a. | n.a. | 0.151 |
| | Donor | | | | | | | |
| | Sepsis vs. | 0.324 | 0.345 | 0.082 | 0.018 | n.a. | n.a. | 0.664 |
| | SIRS | | | | | | | |
| | Sepsis vs. | 0.685 | 0.133 | 0.487 | 0.003 | n.a. | 0.084 | 0.001 |
| | Donor | | | | | | | |

| | | Protein from gene | | | | |
|---|---|---|---|---|---|---|
| | | HLA-DPA1 | | CLU | HLA-DR | |
| | | Cell type | | | | |
| | | T-Lympho- | Mono- | Whole blood | T-Lympho- | Mono- |
| | | cytes | cytes | [Ratio for β | cytes | cytes |
| Sample number | | [%] | [%] | Actin]] | [%] | [%] |
| Donor | 1 | 22.4 | 26.6 | 0.51 | n.a. | n.a. |
| | 2 | 12.7 | 45.3 | 0.54 | n.a. | n.a. |
| | 3 | 33.1 | 77.3 | n.a. | n.a. | n.a. |
| | 4 | 24.6 | 51 | 0.59 | 15.8 | 44.6 |
| | 5 | 31.3 | 35.4 | 0.37 | 16.3 | 57.6 |
| | 6 | 34.5 | 38 | n.a. | 22 | 58.8 |
| | 7 | 24.6 | 49 | n.a. | 11.8 | 67.3 |
| SIRS | 8 | 15 | 34 | n.a. | 17.1 | 17 |
| | 9 | 13.1 | 24.9 | n.a. | 7.2 | 7.8 |
| | 10 | 35.8 | 40.5 | n.a. | 26.3 | 7.8 |
| severe | 11 | 43.2 | 0 | n.a. | n.a. | n.a. |
| sepsis/ | 12 | 50 | 32.1 | n.a. | n.a. | n.a. |
| septic | 13 | 52.9 | 16.5 | 0.35 | 8.31 | 5.19 |
| shock | 14 | 42.3 | 82.6 | 0.4 | 23 | 27.1 |
| | 15 | 82.3 | 19.4 | n.a. | 2.91 | 2.89 |
| | 16 | 17.8 | 5 | n.a. | 15.6 | 4.9 |
| | 17 | 18.8 | 25.5 | n.a. | 6.6 | 15 |
| | 18 | 30.2 | 23.3 | n.a. | 11.5 | 6.1 |
| | 19 | 40.7 | 61.7 | n.a. | 14.4 | 45.1 |
| Average | Donor | 26.2 | 46.1 | 0.5 | 16.48 | 57.08 |
| value | SIRS | 21.3 | 33.1 | n.a. | 16.87 | 10.87 |
| | Sepsis | 42.0* | 29.6 | 0.38 | 11.76 | 15.18 |
| P value | Anova | 0.07 | 0.325 | 0.153 | 0.427 | 0.000 |
| | SIRS vs. | 0.582 | 0.129 | n.a. | 0.952 | 0.001 |
| | Donor | | | | | |
| | Sepsis vs. | 0.081 | 0.728 | n.a. | 0.464 | 0.536 |
| | SIRS | | | | | |
| | Sepsis vs. | 0.048 | 0.148 | 0.153 | 0.185 | 0.000 |
| | Donor | | | | | |

### SUMMARY

The example described above makes clear that the severity of the host response in case of an acute inflammation also is reflected in the expression of proteins from selected genes. The results of gene expression analysis provide for an extensive collection of marker candidates. It is therefore obvious to determine a suitable severity score from protein expression, which sufficiently resembles the master score from the gene expression introduced in example 1.

### LITERATURE

ACCP/SCCM (1992), Crit. Care Med 20, 864-74
Altschul S F, Gish W, Miller W, Myers E W, Lipman D J. (1990) Basic local alignment search tool. J Mol. Biol. October 5; 215(3):403-10.
Benson D. A., Karsch-Mizrachi I., Lipman D. J., Ostell J., Sayers E. W. (2009) GenBank. Nucleic Acids Res. 37(Database issue):D26-31.
Boldrick et al. (2002): Stereotyped and specific gene expression programs in human innate immune responses to bacteria, PNAS 99, 972-977.
Bone R C, Balk R A, Cerra F B, Dellinger E P, Fein A M, Knaus W A, Schein R M, Sibbald W J, the ACCP/SCCM Consensus Conference Committee (1992) Definitions for Sepsis and organ failure and guidelines for the use of innovative therapies in Sepsis. Chest 101, 1656-1662
Brun-Buisson C, Doyon F, Carlet J, Dellamonica P, Gouin F, Lepoutre A, Mercier J C, Offenstadt G, Regnier B (1995) Incidence, risk factors, and outcome of severe Sepsis and septic shock in adults. A multicenter prospective study in intensive care units. French ICU Group for Severe Sepsis. JAMA 274, 968-974
Brun-Buisson C, Roudot-Thoraval F, Girou E, Grenier-Sennelier C, Durand-Zaleski I (2003) The costs of septic syndromes in the intensive care unit and influence of hospital-acquired Sepsis. Intensive Care Med 29, 1464-1471
Bustin S A. (2002) Quantification of mRNA using real-time reverse transcription PCR (RT-PCR): trends and problems. J Mol. Endicronol. 29:23-29.
Calandra T, Cohen J. (2005) International Sepsis Forum Definition of Infection in the ICU Consensus Conference. Critical Care Med. 33(7):1538-48.
Calvano S E, Xiao W, Richards D R, Felciano R M, Baker H V, Cho R J, Chen R O, Brownstein B H, Cobb J P, Tschoeke S K, Miller-Graziano C, Moldawer L L, Mindrinos M N, Davis R W, Tompkins R G, Lowry S F (2005). Nature 437: 10327.
Centers for Disease Control (CDC). (1990) Increase in National Hospital Discharge Survey rates for septicemia—United States, 1979-1987. MMWR Morb Mortal Wkly Rep. 19; 39(2):31-4.
Carrigan S D Scott G Tabrizian M (2004) Toward resolving the challenges of Sepsis. Clin Chem 50(8), 1301-1314
Dagan R, Shriker O, Hazan I, Leibovitz E, Greenberg D, Schlaeffer F, Levy R. (1998) Prospective study to determine clinical relevance of detection of pneumococcal DNA in sera of children by PCR, J Clin Microbiol. 36:669-73.
Davenport P and Land K J. (2007) Isolation of Leclercia adecarboxylata from the blood culture of an asymptomatic platelet donor. Transfusion 47:1816-9.
Du P, Kibbe W, Lin S (2008). Bioinformatics 24(13):1547-1548
FDA: In Vitro Diagnostic Multivariate Index Assays. Draft Guidance for Industry, Clinical Laboratories, and FDA Staff (2007) http://www.fda.gov/downloads/MedicalDevices/DeviceRegulationandGuidance/GuidanceD ocuments/ucm071455.pdf
Feezor R J, Oberholzer C, Baker H V, Novick D, Rubinstein M, Moldawer L L, Pribble J, Souza S, Dinarello C A, Ertel W, Oberholzer A. (2003) Molecular characterization of the acute inflammatory response to infections with gram-negative versus gram-positive bacteria. Infect Immun. 71(10):5803-13.
Feezor R J, Baker H V, Xiao W et al. (2004) Genomic and Proteomic Determinants of Outcome in Patients Untergoing Thoracoabdominal Aortic Aneurysm Repair. J Immun 172, 7103-7109
Fine J M, Fine M J, Galusha D, Petrillo M, Meehan T P. (2002) Patient and hospital characteristics associated with recommended processes of care for elderly patients hospitalized with pneumonia: results from the medicare quality indicator system pneumonia module. Arch. Intern. Med. 162:827-833.
Fleige S, Pfaffl M W (2006). Mol Aspects Med 27: 126-139
Foteinou P T, Calvano S E, Lowry S F, Androulakis I P (2009) Mathematical Biosciences 217: 27-42
Garnacho-Montero J, Garcia-Garmendia J L, Barrero-Almodovar A, Jimenez-Jimenez F J, Perez-Paredes C, Ortiz-Leyba C. (2003) Impact of adequate empirical antibiotic therapy on the outcome of patients admitted to the intensive care unit with sepsis. Crit. Care Med. 31:2742-2751.
Goyert S M, Ferrero E, Rettig W J, Yenamandra A K, Obata F, Le Beau M M (1988). The CD14 monocyte differentiation antigen maps to a region encoding growth factors and receptors. Science 29; 239(4839):497-500
Goutte C, Toft P, Rostrup E, Nielsen F, Hansen L K (1999). Neuroimage, 9(3):298-310
Hartigan, J A, Wong, M A (1979). Applied Statistics 28, 100-108
Heffner A C, Horton J M, Marchick M R, Jones A E (2010) Etiology of Illness in Patients with Severe Sepsis Admitted to the Hospital from the Emergency Department. CID 50:814-820.
Hotchkiss R S, Coopersmith C M, McDunn J E, Ferguson T A. (2009) The sepsis seesaw: tilting toward immunosuppression. Nat. Med. 15(5):496-7.
Hotchkiss R S, Opal S. (2010) Immunotherapy for sepsis—a new approach against an ancient foe. N Engl J. Med. 363(1):87-9.
Huber W, von Heydebreck A, Sueltmann H, Poustka A, Vingron M (2002) Bioinformatics 18 Supp1.1:96-5104
Huggett J, Dheda K, Bustin S, Zumla A. (2005) Real-time RT-PCR normalisation: strategies and considerations. Genes Immun. 6(4):279-284.
Ibrahim E H, Sherman G, Ward S, Fraser V J, Kollef M H. The influence of inadequate antimicrobial treatment of bloodstream infections on patient outcomes in the ICU setting. Chest. 2000; 118(1):146-55.
Inoue S, Unsinger J, Davis C G, Muenzer J T, Ferguson T A, Chang K, Osborne D F, Clark A T, Coopersmith C M, McDunn J E, Hotchkiss R S. (2010) IL-15 prevents apoptosis, reverses innate and adaptive immune dysfunction, and improves survival in sepsis. J. Immunol. 184(3):1401-9.
Iregui, M., Ward, S., Sherman, G., Fraser, V. J., Kollef, M. H. (2002) Clinical importance of delays in the initiation of appropriate antibiotic treatment for ventilator-associated pneumonia. Chest 122(1):262-268.
Isaacman, D. J. et al., Accuracy of a polymerase chain reaction-based assay for detection of pneumococcal bacteremia in children, Pediatrics, 1998; 101:813-6.
Johnson S B, Lissauer M, Bochicchio G V, Moore R, Cross A S, Scalea T M. (2007) Gene Expression Profiles Differentiate Between Sterile SIRS and Early Sepsis Annals of Surgery 245(4), 611-621
Koulaouzidis A, Bhat S, Saeed A A (2009) Spontaneous bacterial peritonitis. World J. Gastroenterol. 15(9):1042-9.
Increase in National Hospital Discharge Survey rates for septicemia—United States, 1979-1987. MMWR Morb Mortal Wkly Rep 1990 39, 31-34
Kimura F, Shimizu H, Yoshidome H, Ohtsuka M, Miyazaki M. (2010) Immunosuppression following surgical and traumatic injury. Surg. Today 40(9):793-808.
Klein D. (2002) Quantification using real-time PCR technology: applications and limitations. Trends Mol. Med. 8(6):257-260.
Kofoed K, Andersen O, Kronborg G, Tvede M, Petersen J, Eugen-Olsen J, Larsen K (2007), Use of plasma C-reactive protein, procalcitonin, neutrophils, macrophage migration inhibitory factor, soluble urokinase-type plasminogen activator receptor, and soluble triggering receptor expressed on myeloid cells-1 in combination to diagnose infections: a prospective study. Critical Care 11, R38
Kumar A, Roberts D, Wood K E et al. (2006) Duration of hypothension before initiation of effective antimicrobial therapy is the critical determinant of survival in human septic shock. Crit. Care Med 34(6), 1589-1596
Le-Gall J R, Lemeshow S, Leleu G, Klar J, Huillard J, Rue M, Teres D, Artigas A (1995) Customized probability models for early severe Sepsis in adult intensive care patients. Intensive Care Unit Scoring Group. JAMA 273, 644-650
Levy M M, Fink M P, Marshall J C, Abraham E, Angus D, Cook D, Cohen J, Opal S M, Vincent J L, Ramsay G et al. (2003) 2001 SCCM/ESICM/ACCP/ATS/SIS International Sepsis Definitions Conference. Intensive Care Med 29, 530-538
Mardia, K V, Kent, J T, Bibby, J M (1979): Multivariate Analysis, New York 1979
Marshall J C, Foster D, Vincent J L, Cook D J, Cohen J, Dellinger R P, Opal S, Abraham E, Brett S J, Smith T, Mehta S, Derzko A, Romaschin A; MEDIC study. (2004) Diagnostic and prognostic implications of endotoxemia in critical Illness: Results of the MEDIC Study. J Infect Dis. 190(3):527-34.
Meisel C, Schefold J C, Pschowski R, Baumann T, Hetzger K, Gregor J, Weber-Carstens S, Hasper D, Keh D, Zuckermann H, Reinke P, Volk H D. (2009) Granulocyte-macrophage colony-stimulating factor to reverse sepsis-associated immunosuppression: a double-blind, randomized, placebo-controlled multicenter trial.
Am J Respir Crit. Care Med. 180(7):640-8.
Monneret G, Venet F, Pachot A, Lepape A. (2008) Monitoring Immune dysfunctions in the Septic Patient: A New Skin for the Old Ceremony, Mol. Med. 14(1-2):64-78.
Monneret G, Venet F, Meisel C, Schefold J C. (2010) Assessment of monocytic HLA-DR expression in ICU patients: analytical issues for multicentric flow cytometry studies. Crit. Care 14(4):432.
Muenzer J T, Davis C G, Chang K, Schmidt R E, Dunne W M, Coopersmith C M, Hotchkiss R S. (2010) Characterization and modulation of the immunosuppressive phase of sepsis. Infect Immun. 78(4):1582-92.
Nolan T, Hands R E, Bustin S A. (2006) Quantification of mRNA using realt-time RT-PCR. Nat. Protoc. 1(3):1559-1582.
Opal S M, Lim Y-P, Siryaporn E, et al. (2005) Longitudinal studies of inter-alpha inhibitor proteins in severly septic patients: A potential clinical marker and mediator of severe sepsis. Clin Invest 35(2), 387-292
Pachot A, Lepape A, Vey S, et al. (2006) Systemic transcriptional analysis in survivor and non-survivor septic shock patients: a preliminary study. Immunol Lett 106(1), 63-71. Epub 2006 May 17
Pierrakos C, Vincent J L. (2010) Sepsis biomarkers: a review, Crit. Care 14:R15.
Pruitt K. D., Tatusova T., Maglott D. R. (2007) NCBI reference sequences (RefSeq): a curated non-redundant sequence database of genomes, transcripts and proteins. Nucleic Acids Res. 35 (Database issue):D61-5.
Rodero L, Cuenca-Estrella M, Córdoba S, Cahn P, Davel G, Kaufman S, Guelfand L, Rodriguez-Tudela J L. (2002) Transient fungemia caused by an amphotericin B-resistant isolate of Candida haemulonii. J Clin Microbiol. 40:2266-9.
R Development Core Team (2006), Vienna, Austria. ISBN 3-900051-07-0, URL http://www.R-project.org
Ramilo O, Allman W, Chung W, Mejias A, Ardura M, Glaser C, Wittkowski K M, Piqueras P, Bancherau J, Palucka K A, Chaussabel D, (2007) Gene expression patterns in blood leukocytes discriminate patients with acute infections. Blood 109, 2066-2077
Ruokonen E et al. (1999) Procalcitonin concentrations in patients with neutropenic fever. Eur J Clin Microbiol Infect Dis 18(4), 283-5
Ruokonen E et al. (2002) Procalcitonin and neopterin as indicators of infection in critically ill patients. Acta Anaesthesiol Scand 46(4), 398-404
Schrenzel J. (2007) Clinical relevance of new diagnostic methods for bloodstream infections. Int J Antimicrob Agents. 30 Suppl 1:S2-6.
Simon L, Gauvin F, Amre D K, Saint-Lois P, Lacroix J, 2004) Serum procalcitonin and C-reactive protein levels as marker of bacterial infection: A systematic review and meta analysis. Clin Infec Dis 39, 206-217
Simon R. (2005) Roadmap for Developing and Validating Therapeutically Relevant Genomic Classifiers. J Clin Oncol 23, 7332-7341
Sponholz C, Sakr Y, Reinhart K, Brunkhorst F, (2006) Diagnostic value and prognostic implications of serum procalcitonin after cardiac surgery: a systematic review of the literature, Critical Care 10, R145
Storey J D, Tibshirani R (2003). PNAS 100 (16): S9440-S9445
Strimmer, K. (2008). Bioinformatics 24: 1461-1462
Suprin E et al. (2000) Procalcitonin: a valuable indicator of infection in a medical ICU Intensive Care Med 26(9), 1232-1238
Tang B M P, Eslick G D, Craig J C et al. (2007a) Accuracy of procalcitonin for sepsis diagnosis in critically ill patients: systematic review and meta-analysis. Lancet Infect Dis 7, 210-217
Tang B M P, McLean A S, Dawes I W, Huang S J, Lin R C Y (2007b) The Use of Gene-Expression Profiling to Identify Candidate Genes In Human Sepsis, American Journal of Respiratory and Critical Care Medicine 176(7), 676-684
Tang B M, Huang S J, McLean A S (2010). Critical Care, 14:R237, doi:10.1186/cc9392
Tsoukas C D, Landgraf B, Bentin J, Valentine M, Lotz M, Vaughan J H, Carson D A (1985). Activation of resting T lymphocytes by anti-CD3 (T3) antibodies in the absence of monocytes. J. Immunol. 135(3):1719-23
The NCBI handbook. Bethesda (MD): National Center for Biotechnology Information (US) 2002-.
Torgersen C, Moser P, Luckner G, Mayr V, Jochberger S, Hasibeder WR, Dünser MW. (2009), Macroscopic postmortem Findings in 235 surgical intensive care patients with sepsis. Critical Care and Trauma 108(6):1841-7.
US 2010/0293130 A1
US 2009/0307181 A1
US 2008/0020379 A1
US 20060246495
U.S. Pat. No. 6,960,439
Valasek M A, Repa J J. (2005) The power of real-time PCR. Advan Physiol Educ. 29:151-159.
Valles, J., Rello, J., Ochagavia, A., Garnacho, J., Alcala, M. A. (2003) Community-acquired bloodstream infection in critically ill adult patients: impact of shock and inappropriate antibiotic therapy on survival. Chest 123:1615-1624.
Venet F, Davin F, Guignant C, Larue A, Cazalis M A, Darbon R, Allombert C, Mougin B, Malcus C, Poitevin-Later F, Lepape A, Monneret G. (2010) Early assessment of leukocyte alterations at diagnosis of septic shock. Shock 34(4):358-63.
Warren et al. (2009): A Genomic Score Prognostic of Outcome in Trauma Patients, Mol Med 15, 220-227.
WO 2006/100203
WO 2004/087949
WO 2005/083115
WO 2005/106020
WO 2006/042581
WO 2007/144105
WO 2007/124820
WO 2003/084388
Wong M L, Medrano J F (2005) Real-time PCR for mRNA quantification. Biotechniques 39(1):1-11.

1-22. (canceled)

23. A method for identifying a subset of polynucleotides from an initial set of polynucleotides corresponding to a transcriptome for the in vitro determination of the severity of the host response of a patient being in an acutely infectious and/or acutely inflammatory condition, in a sample, a measuring device being used that comprises a plurality of different gene probes that essentially represent the entire human genome, wherein
samples of nucleic acid of a plurality of test persons exhibiting a known phenotypic physiological condition, are brought into contact with the probes of the measuring device so as to obtain signals of the respective expression of a gene;
of the total number of gene probes used those are selected that provide an expression signal of detectable intensity for at least one sample of nucleic acid of a test person;
the test persons, depending on their infectious and/or inflammatory status, are divided into at least two of the following clinically determined phenotype groups:
Inflammation

| | Systemic | Local | None |
|---|---|---|---|
| Infection | [S] | [L] | [N] |
| Systemic [S] | SaS | | |
| Local [L] | LaS | LaL | |
| None [N] | NaS | NaL | NaN |

wherein a“ represents an AND-operation between the properties S, L and N;
the changes of the gene expression signals between the groups of phenotypes are compared statistically and it is assessed as to whether there is a significant difference between at least two of the phenotype groups;
those gene probes are selected from the gene expression signals of which have significantly changed statistically between at least two phenotype groups and an estimated number of those gene probes is excluded that provide a false positive result in relation to a predetermined threshold value;
a master score is determined as measurement for the severity of the host response of a test person being in an acutely infectious and/or acutely inflammatory condition, by quantifying an increase and decrease in the gene expression intensity of the selected gene probes; and
compared to the initial set, a considerably reduced number of polynucleotides is identified by determining a score that comprises at most a predetermined deviation from the master score and that likewise serves as a measurement for the severity of the host response of a test person being in an acutely infectious and/or acutely inflammatory condition.

24. The method of claim 23, characterized in that a measuring device is used that comprises 20,000 to 50,000 gene probes, and/or that the measuring device is a bio chip with matrix-shaped immobilized gene probes thereon, wherein each individual gene probe is associated one-to-one with location coordinates on said bio chip.

25. The method of claim 23, characterized in that the expression signals are obtained by way of hybridization and/or amplification, in particular PCR, preferably quantitative PCR, preferably real time PCR and/or evidence of protein.

26. The method of claim 23, characterized in that the 6 phenotype groups from table 1 are compared with each other in pairs.

27. The method of claim 23, characterized in that a statistically significant difference between groups is determined of which at least one group includes more than one phenotype group.

28. The method of claim 23, characterized in that a predetermined threshold value for an estimated number of gene probes providing a false positive result is within a range of 0.1 to 5%, particularly within a range of 0.3% of the number of gene probes used, and that the estimated number of false positive gene probes is excluded.

29. The method of claim 23, characterized in that the relative distance of the gene expression signals with regard to an Euclidean distance between an average value mS of phenotype group SaS and an average value mH of phenotype group NaN servers as master score.

30. The method of claim 29, characterized in that the relative distance is obtained as follows:
$$score ( X ) [ \% ] = \frac{100 \%}{d ( mS , mH )} \cdot cor ( X - mH , mS - mH ) d ( X , mS - mH )$$
wherein d(X₁, X₂) denotes the Euclidean distance and cor(X₁, X₂) denotes the correlation coefficient according to Pearson between the two vectors X₁ and X₂.

31. The method of claim 23, characterized in that the score is to deviate upward and downward from the master score by at most 5 to 15 percentage points, in particular 10 percentage points.

32. The method of claim 23, characterized in that the master score is formed from the group of polynucleotides with SEQ ID No: 1 to SEQ ID No: 7704.

33. The method of claim 23, characterized in that a series of score values is measured, each at different points of time, so as to determine the time-dependent course of the severity of the host response of a patient being in an acutely infectious and/or inflammatory condition.

34. The method of claim 23, characterized in that the acutely infectious condition comprises at least one of the following patho-physiological conditions selected from the group consisting of abscesses, bacteremia, postoperative infections, wound infections, local infections, systemic infections, sepsis, severe sepsis, and septic shock.

35. The method of claim 23, characterized in that the acutely inflammatory condition comprises at least one of the following pathophysiological conditions selected from the group consisting of traumata, burns, radiation injuries, toxic injuries, ischemia/reperfusion injuries, acute rejection reactions, inflammatory bowel diseases, oncologic diseases, post-operative conditions, local inflammation, systemic inflammation, SIRS, allergic reaction, and anaphylactic shock.

36. A method of use of k tuples of polynucleotides that are selected from the group consisting of m polynucleotides with SEQ ID No: 1 to SEQ ID No: 7704, wherein k is at least 7 and equal to or smaller than the number of polynucleotides m in the group and at least one of the subsequent subsets of polynucleotides is used, “n” indicating the number of polynucleotides of the respective set; for assessing a score as measurement of the severity of the host response of a test person being in an acutely infectious and/or acutely inflammatory condition:
Set 1 (n=49) SEQ ID No: 508, 553, 611, 679, 734, 769, 851, 860, 871, 896, 1117, 1263, 1646, 1647, 1648, 1675, 1688, 1975, 2011, 2077, 2415, 2516, 2560, 2581, 3381, 3491, 3820, 3947, 4156, 4230, 4506, 4576, 5012, 5235, 5614, 5730, 5803, 5873, 6114, 6262, 6265, 6301, 6689, 6738, 6820, 6847, 6879, 7069, and 7230;
Set 2 (n=47) SEQ ID No: 160, 309, 374, 428, 462, 911, 937, 1039, 1092, 1105, 1458, 1533, 1604, 1895, 1917, 1997, 2002, 2055, 2242, 2332, 2369, 2386, 2427, 2516, 2541, 2560, 2785, 3359, 3407, 3624, 4230, 4587, 4636, 5164, 5235, 5247, 5371, 5776, 6278, 6328, 6497, 6636, 7156, 7201, 7230, 7314, and 7450;
Set 3 (n=48) SEQ ID No: 10, 366, 411, 462, 493, 495, 567, 1204, 1226, 1409, 1414, 1449, 1487, 1583, 1724, 1744, 2013, 2055, 2064, 2208, 2248, 2692, 2891, 3051, 3624, 4156, 4205, 4510, 4587, 4923, 5176, 5373, 5400, 5435, 5873, 5912, 5954, 6041, 6073, 6247, 6301, 6478, 6525, 6923, 7207, 7450, 7670, and 7681;
Set 4 (n=47) SEQ ID No: 160, 359, 441, 493, 522, 541, 652, 691, 1128, 1408, 1583, 1651, 1652, 1664, 1688, 2002, 2077, 2248, 2273, 2415, 2676, 2690, 2755, 2876, 3053, 3623, 4216, 4327, 4525, 4587, 4765, 4870, 5013, 5164, 5431, 5614, 5950, 6098, 6265, 6432, 6497, 6981, 7062, 7202, 7314, 7450, and 7607;
Set 5 (n=49) SEQ ID No: 97, 428, 441, 543, 611, 851, 1136, 1384, 1533, 1868, 1997, 2077, 2183, 2208, 2226, 2260, 2329, 2386, 2475, 2686, 2690, 2876, 3054, 3821, 4000, 4357, 4479, 4530, 4636, 4765, 4923, 5013, 5137, 5204, 5760, 5776, 5819, 5873, 5908, 6005, 6099, 6242, 6417, 6499, 6585, 6847, 7450, 7670, and 7681;
Set 6 (n=48) SEQ ID No: 10, 97, 359, 475, 495, 627, 928, 1039, 1117, 1248, 1384, 1408, 1472, 1652, 1675, 1744, 1868, 1918, 2370, 2423, 2537, 2742, 2865, 3051, 3086, 3408, 3916, 4030, 4078, 4274, 4294, 4362, 4751, 5129, 5235, 5247, 5431, 5734, 5803, 5811, 5908, 5950, 6005, 6417, 6497, 6525, 6923, and 7456;
Set 7 (n=49) SEQ ID No: 32, 160, 383, 414, 493, 611, 652, 679, 734, 885, 896, 946, 1177, 1640, 1650, 1704, 1882, 2077, 2248, 2250, 2260, 2415, 2561, 3086, 3488, 3623, 3624, 4135, 4156, 4160, 4510, 4525, 4530, 4742, 5137, 5204, 5247, 5730, 5950, 6114, 6210, 6225, 6430, 6478, 6497, 6545, 6668, 7314, and 7607;
Set 8 (n=48) SEQ ID No: 359, 383, 515, 538, 544, 691, 769, 813, 1024, 1039, 1092, 1409, 1519, 1640, 1649, 1665, 1696, 1731, 1744, 2167, 2183, 2226, 2260, 2273, 2425, 2516, 2618, 2634, 2672, 3051, 3168, 3202, 4160, 4754, 4966, 5373, 5465, 5493, 5541, 5574, 5912, 6005, 6216, 6432, 6636, 6748, 6847, and 7423;
Set 9 (n=46) SEQ ID No: 160, 352, 544, 691, 802, 885, 1126, 1147, 1163, 1336, 1416, 1639, 1969, 2002, 2058, 2077, 2183, 2331, 2332, 2426, 2526, 2742, 2855, 2860, 2891, 3054, 3138, 3488, 3947, 4560, 4576, 4707, 4776, 5235, 5371, 5400, 5431, 5760, 5873, 6247, 6301, 6417, 6673, 6820, 7447, and 7604;
Set 10 (n=49) SEQ ID No: 8, 164, 462, 494, 495, 510, 545, 567, 611, 679, 941, 1039, 1105, 1128, 1147, 1318, 1533, 1649, 1918, 1973, 1975, 2011, 2077, 2080, 2370, 2537, 3051, 3202, 3676, 4274, 4587, 4928, 5204, 5373, 5431, 5465, 5541, 5734, 5908, 5912, 5950, 6278, 6417, 6497, 6668, 6673, 7156, 7230, and 7670;
Set 11 (n=46) SEQ ID No: 89, 97, 160, 355, 359, 366, 374, 411, 462, 475, 515, 538, 543, 691, 1384, 1647, 1649, 1651, 1724, 2011, 2058, 2064, 2242, 2369, 2859, 3414, 4000, 4742, 4765, 4870, 4966, 5040, 5232, 5247, 5276, 5373, 5431, 5760, 5873, 5954, 6417, 6419, 6497, 6545, 6636, and 7484;
Set 12 (n=49) SEQ ID No: 8, 89, 515, 543, 585, 769, 969, 1126, 1163, 1526, 1583, 1639, 1744, 2019, 2393, 2415, 2453, 2618, 2690, 2692, 2810, 2855, 2863, 3153, 3158, 3190, 3408, 4000, 4083, 4104, 4248, 4479, 4491, 4550, 4661, 4877, 4995, 5176, 5276, 5599, 5695, 6073, 6114, 6265, 6417, 6499, 6585, 6632, and 6673;
Set 13 (n=48) SEQ ID No: 414, 538, 946, 1263, 1384, 1512, 1895, 2077, 2248, 2260, 2516, 2676, 2975, 3168, 3414, 4083, 4274, 4776, 4800, 4919, 4923, 5179, 5204, 5431, 5493, 5541, 5619, 5695, 5819, 6005, 6073, 6099, 6210, 6247, 6265, 6350, 6417, 6432, 6499, 6536, 6545, 6636, 6668, 6689, 7040, 7062, 7472, and 7604;
Set 14 (n=47) SEQ ID No: 383, 428, 538, 553, 691, 814, 871, 896, 911, 937, 1426, 1639, 1685, 1688, 1983, 2093, 2253, 2260, 2454, 2516, 2587, 2672, 2761, 2865, 2975, 3086, 3781, 4000, 4030, 4308, 4510, 4636, 4923, 5137, 5235, 5574, 5776, 5819, 5908, 6226, 6278, 6417, 6632, 7202, 7230, 7315, and 7456;
Set 15 (n=46) SEQ ID No: 97, 366, 383, 802, 1426, 1514, 1558, 1685, 1744, 1975, 2011, 2013, 2369, 2415, 2454, 2510, 2516, 2577, 2587, 2759, 2968, 3168, 3364, 3641, 3780, 4083, 4230, 4294, 4587, 4638, 4817, 5040, 5164, 5276, 5371, 5465, 5541, 6073, 6098, 6114, 6184, 6216, 6497, 6515, 7062, and 7202;
Set 16 (n=49) SEQ ID No: 10, 504, 541, 553, 567, 652, 802, 1024, 1092, 1136, 1197, 1519, 1646, 1647, 1648, 1652, 2055, 2058, 2260, 2273, 2330, 2331, 2415, 2491, 2581, 2618, 2676, 2742, 3053, 3408, 3652, 3915, 4216, 4870, 5235, 5641, 5695, 5954, 6114, 6278, 6419, 6461, 6791, 6820, 6847, 6923, 7428, 7604, and 7670;
Set 17 (n=20) SEQ ID No: 10, 160, 428, 871, 941, 1136, 1197, 1416, 1558, 1786, 1951, 2386, 2510, 2560, 3488, 3652, 3781, 5176, 5400, and 6515;
Set 18 (n=20) SEQ ID No: 871, 1163, 1414, 1416, 1426, 1487, 2001, 2055, 2369, 2386, 2552, 2577, 2865, 3051, 4550, 4577, 5614, 6098, 7369, and 7423;
Set 19 (n=20) SEQ ID No: 567, 958, 2226, 2250, 2260, 2427, 2516, 3364, 4030, 4135, 5235, 5574, 5950, 6114, 6226, 6267, 6278, 6418, 7069, and 7518;
Set 20 (n=20) SEQ ID No: 409, 1647, 1648, 1770, 1883, 1951, 2013, 2386, 2423, 3152, 3491, 4205, 4577, 4661, 4765, 4919, 7428, and 7604;
Set 21 (n=20) SEQ ID No: 355, 480, 494, 667, 1492, 2475, 2855, 2948, 3155, 3158, 3408, 3780, 4661, 5113, 5232, 5368, 5574, 6114, 6419, and 6499;
Set 22 (n=19) SEQ ID No: 769, 1163, 1472, 2077, 2370, 2759, 3488, 3567, 3737, 3780, 4230, 4245, 4274, 4550, 5950, 6497, 7069, 7109, and 7681;
Set 23 (n=20) SEQ ID No: 160, 164, 355, 411, 1106, 1408, 1675, 1679, 2386, 2453, 2516, 2810, 3168, 3202, 3652, 4230, 5574, 5986, 7428, and 7484;
Set 24 (n=19) SEQ ID No: 10, 164, 885, 1263, 1318, 1416, 1492, 1508, 1647, 1951, 2250, 2560, 2785, 2827, 3086, 4506, 5137, 5575, and 5954;
Set 25 (n=19) SEQ ID No: 32, 522, 679, 1519, 2001, 2491, 2516, 2676, 3412, 3737, 4205, 4294, 4560, 5235, 5954, 6005, 6114, 6499, and 6525;
Set 26 (n=19) SEQ ID No: 958, 1449, 1472, 1582, 2332, 2516, 2552, 2891, 2975, 3168, 3190, 3683, 3820, 3947, 4245, 4530, 7040, 7069, and 7145;
Set 27 (n=20) SEQ ID No: 428, 515, 544, 562, 567, 1263, 2002, 2332, 2526, 3438, 4577, 4754, 5574, 5614, 5912, 6328, 6515, 7156, 7423, and 7456;
Set 28 (n=20) SEQ ID No: 355, 508, 937, 1263, 1973, 2002, 2510, 4078, 4156, 4550, 4673, 4817, 5247, 5368, 5730, 6005, 6247, 6515, 7201, and 7207;
Set 29 (n=20) SEQ ID No: 10, 544, 871, 1408, 1487, 1649, 2002, 2415, 2690, 2859, 2975, 3126, 4577, 4636, 5541, 6073, 6417, 6432, 6866, and 6879;
Set 30 (n=20) SEQ ID No: 896, 1248, 1318, 1472, 1786, 1830, 1983, 2386, 2865, 2975, 3641, 3916, 4030, 4530, 4995, 5472, 5619, 6099, 6247, and 6265;
Set 31 (n=20) SEQ ID No: 355, 462, 1416, 1983, 2011, 2183, 2248, 2618, 3190, 3412, 4490, 4576, 4776, 4923, 5164, 6101, 6114, 6278, 7314, and 7369;
Set 32 (n=20) SEQ ID No: 310, 1226, 1895, 2248, 2427, 2516, 2552, 2690, 3086, 3438, 3915, 4216, 4587, 5235, 5276, 5954, 6265, 6478, 6515, and 7207;
Set 33 (n=20) SEQ ID No: 493, 584, 633, 937, 2330, 2377, 2491, 2587, 3153, 3683, 4216, 4248, 4530, 6114, 6419, 6478, 6525, 6689, 7202, and 7456;
Set 34 (n=20) SEQ ID No: 10, 359, 383, 478, 626, 1472, 1487, 1647, 2475, 3683, 3780, 4490, 4636, 5179, 5247, 5371, 5950, 6748, 6923, and 7670;
Set 35 (n=20) SEQ ID No: 97, 626, 1039, 1163, 1426, 1617, 1704, 2002, 2248, 2690, 3168, 4216, 4638, 5247, 5614, 5950, 6265, 6461, 6632, and 7428;
Set 36 (n=20) SEQ ID No: 366, 414, 544, 734, 1263, 1416, 2167, 2208, 2250, 2370, 2491, 2526, 2855, 3190, 3488, 4083, 4248, 6673, 6845, and 6847;
Set 37 (n=10): SEQ ID No: 1983, 507, 5431, 3043, 1665, 5776, 2902, 6585, 3167 and 745; and
Set 38 (n=7): SEQ ID No: 1983, 507, 5431, 3043, 1665, 5776 and 7695.

37. The method of claim 36, characterized in that isoforms of the polynucleotides are used, particularly such isoforms that are selected from the group consisting of polynucleotides with SEQ ID No: 507, 7695, 7696, 7697, 7698, 7699, 7700, 7701; 5431, 4636; 7702, 7703, and 7704.

38. The method of claim 36, characterized in that fragments of the polynucleotides are used, particularly those with lengths of 20 to 1,000, in particular 15 to 500 nucleotides, preferably 15 to 200, particularly preferably 20 to 200 nucleotides and/or fragments having a sequence homology with regard to the polynucleotides of at least approximately 20%, preferably approximately 50%, and a particularly preferred sequence homology of approximately 80%.

39. The method of claim 36, characterized in that the score is assessed via expression signals, said expression signals being obtained by way of hybridization and/or amplification, in particular PCR, preferably quantitative PCR, preferably real time PCR and/or evidence of protein.

40. The method of claim 36, characterized in that the score is used for diagnosis, predicting the development, or monitoring the acutely infectious and/or inflammatory condition of a patient, and/or for controlling the progress of therapy and/or for focus control.

41. The method of claim 36, characterized in that the score is used for indicating the chance of recovery or non-recovery of a patient being in an acutely infectious and/or acutely inflammatory condition.

42. A method of use of a plurality of protein gene products selected from the group consisting of: TLR5, CD59, CPVL, FGL2, IL7R, HLA-DPA1, HLA-DR, and CLU, for assessing a score as measurement of the severity of the host response of a test person being in an acutely infectious and/or acutely inflammatory condition.

43. The method of claim 42, characterized in that k tuples of the protein gene products are used, wherein k is at least 7 and equal to or smaller than the number of polynucleotides m in the group.

44. The method of claim 42, characterized in that such fragments of the proteins are used that have a sequence homology with regard to the proteins of at least approximately 20%, preferably approximately 50%, and a particularly preferred sequence homology of approximately 80%.
